(19) United States
(12) Patent Application Publication
(10) Pub. No.: US 2021/0137455 A1
(43) Pub. Date: May 13, 2021

(54) EEG Eyeglasses and Eyeglass Accessories for Wearable Mobile EEG Monitoring

(71) Applicant: Robert A. Connor, St. Paul, MN (US)

(72) Inventor: Robert A. Connor, St. Paul, MN (US)

(73) Assignee: Medibotics LLC, St. Paul, MN (US)

(21) Appl. No.: 17/136,117

(22) Filed: Dec. 29, 2020

Related U.S. Application Data
(63) Continuation-in-part of application No. 16/838,541, filed on Apr. 2, 2020.
Application No. 16/838,541 is a continuation-in-part of application No. 16/737,052, filed on Jan. 8, 2020.
Application No. 16/737,052 is a continuation-in-part of application No. 16/568,580, filed on Sep. 12, 2019.
Application No. 16/568,580 is a continuation-in-part of application No. 16/554,029, filed on Aug. 28, 2019.
Application No. 16/554,029 is a continuation-in-part of application No. 16/554,029, filed on Aug. 28, 2019.
Application No. 16/554,029 is a continuation-in-part of application No. 15/236,401, filed on Aug. 13, 2016, now abandoned.
Application No. 16/737,052 is a continuation-in-part of application No. 16/568,580, filed on Sep. 12, 2019.
Application No. 16/568,580 is a continuation-in-part of application No. 15/963,061, filed on Apr. 25, 2018, now Pat. No. 10,772,559.
Application No. 16/568,580 is a continuation-in-part of application No. 15/963,061, filed on Apr. 25, 2018, now Pat. No. 10,772,559.
Application No. 15/963,061 is a continuation-in-part of application No. 16/022,987, filed on Jun. 29, 2018.
Application No. 16/022,987 is a continuation-in-part of application No. 15/136,948, filed on Apr. 24, 2016, now Pat. No. 10,234,942.
Application No. 15/963,061 is a continuation-in-part of application No. 15/464,349, filed on Mar. 21, 2017, now Pat. No. 9,968,297.
Application No. 15/464,349 is a continuation-in-part of application No. 15/136,948, filed on Apr. 24, 2016, now Pat. No. 10,234,942.
Application No. 15/136,948 is a continuation-in-part of application No. 14/599,522, filed on Jan. 18, 2015, now Pat. No. 9,814,426.
Application No. 14/599,522 is a continuation-in-part of application No. 14/562,719, filed on Dec. 7, 2014, now Pat. No. 10,130,277.
Application No. 14/562,719 is a continuation-in-part of application No. 14/330,649, filed on Jul. 14, 2014, now abandoned.
Application No. 15/236,401 is a continuation-in-part of application No. 15/136,948, filed on Apr. 24, 2016, now Pat. No. 10,234,942.
Application No. 15/136,948 is a continuation-in-part of application No. 14/599,522, filed on Jan. 18, 2015, now Pat. No. 9,814,426.
Application No. 14/599,522 is a continuation-in-part of application No. 14/599,522, filed on Jan. 18, 2015, now Pat. No. 9,814,426.
Application No. 14/599,522 is a continuation-in-part of application No. 14/562,719, filed on Dec. 7, 2014, now Pat. No. 10,130,277.
Application No. 14/330,649 is a continuation-in-part of application No. 13/797,955, filed on Mar. 12, 2013, now Pat. No. 9,456,916.
Application No. 13/797,955 is a continuation-in-part of application No. 13/523,739, filed on Jun. 14, 2012, now Pat. No. 9,042,596.
(60) Provisional application No. 62/972,692, filed on Feb. 11, 2020.
(60) Provisional application No. 62/972,692, filed on Feb. 11, 2020.
(60) Provisional application No. 62/851,917, filed on May 23, 2019.
(60) Provisional application No. 62/851,904, filed on May 23, 2019.
(60) Provisional application No. 62/837,712, filed on Apr. 23, 2019.
(60) Provisional application No. 62/851,904, filed on May 23, 2019.
(60) Provisional application No. 62/796,901, filed on Jan. 25, 2019.
(60) Provisional application No. 62/791,838, filed on Jan. 13, 2019.
(60) Provisional application No. 62/430,667, filed on Dec. 6, 2016.
(60) Provisional application No. 62/322,594, filed on Apr. 14, 2016.
(60) Provisional application No. 62/303,126, filed on Mar. 3, 2016.
(60) Provisional application No. 62/169,661, filed on Jun. 2, 2015.
(60) Provisional application No. 62/160,172, filed on May 12, 2015.
(60) Provisional application No. 62/089,696, filed on Dec. 9, 2014.
(60) Provisional application No. 62/017,615, filed on Jun. 26, 2014.
(60) Provisional application No. 61/939,244, filed on Feb. 12, 2014.
(60) Provisional application No. 61/932,517, filed on Jan. 28, 2014.
(60) Provisional application No. 61/932,517, filed on Jan. 28, 2014.
(60) Provisional application No. 61/729,494, filed on Nov. 23, 2012.

(51) Int. Cl. A61B 5/00; G02C 11/00; A61B 5/291

(52) U.S. Cl. CPC A61B 5/6803; A61B 5/0006; A61B 2560/0468; A61B 5/291; G02C 11/10

(57) ABSTRACT

This invention comprises EEG eyeglasses and eyeglass accessories which enable mobile EEG monitoring while being relatively-unobtrusive for use during daily life. An eyeglass accessory device can be a band with EEG sensors which attaches to a person's eyeglasses and loops across the person's forehead, over the person's head, or around the back of the person's head. This band can be transparent. Such a band can also be integrated into smart eyeglasses instead of being an eyeglass accessory.

### CROSS-REFERENCE TO RELATED APPLICATIONS

This application claims the priority benefit of U.S. provisional patent application 62/972,692 filed on 2020 Feb. 11. This application is a continuation-in-part of U.S. patent application Ser. No. 16/838,541 filed on 2020 Apr. 2. This application is a continuation-in-part of U.S. patent application Ser. No. 16/737,052 filed on 2020 Jan. 8. This application is a continuation-in-part of U.S. patent application Ser. No. 16/568,580 filed on 2019 Sep. 12. This application is a continuation-in-part of U.S. patent application Ser. No. 16/554,029 filed on 2019 Aug. 28.

U.S. patent application Ser. No. 16/838,541 claimed the priority benefit of U.S. provisional patent application 62/972,692 filed on 2020 Feb. 11. U.S. patent application Ser. No. 16/838,541 claimed the priority benefit of U.S. provisional patent application 62/851,917 filed on 2019 May 23. U.S. patent application Ser. No. 16/838,541 claimed the priority benefit of U.S. provisional patent application 62/851,904 filed on 2019 May 23. U.S. patent application Ser. No. 16/838,541 claimed the priority benefit of U.S. provisional patent application 62/837,712 filed on 2019 Apr. 23. U.S. patent application Ser. No. 16/838,541 was a continuation-in-part of U.S. patent application Ser. No. 16/554,029 filed on 2019 Aug. 28. U.S. patent application Ser. No. 16/838,541 was a continuation-in-part of U.S. patent application Ser. No. 15/236,401 filed on 2016 Aug. 13.

U.S. patent application Ser. No. 16/737,052 was a continuation-in-part of U.S. patent application Ser. No. 16/568,580 filed on 2019 Sep. 12. U.S. patent application Ser. No. 16/737,052 was a continuation-in-part of U.S. patent application Ser. No. 15/963,061 filed on 2018 Apr. 25 which issued as U.S. patent Ser. No. 10/772,559 on 2020 Sep. 15. U.S. patent application Ser. No. 16/568,580 was a continuation-in-part of U.S. patent application Ser. No. 15/963,061 filed on 2018 Apr. 25 which issued as U.S. patent Ser. No. 10/772,559 on 2020 Sep. 15. U.S. patent application Ser. No. 16/554,029 claimed the priority benefit of U.S. provisional patent application 62/851,904 filed on 2019 May 23. U.S. patent application Ser. No. 16/554,029 claimed the priority benefit of U.S. provisional patent application 62/796,901 filed on 2019 Jan. 25. U.S. patent application Ser. No. 16/554,029 claimed the priority benefit of U.S. provisional patent application 62/791,838 filed on 2019 Jan. 13. U.S. patent application Ser. No. 16/554,029 was a continuation-in-part of U.S. patent application Ser. No. 16/022,987 filed on 2018 Jun. 29.

U.S. patent application Ser. No. 16/022,987 was a continuation-in-part of U.S. patent application Ser. No. 15/136,948 filed on 2016 Apr. 24 which issued as U.S. patent Ser. No. 10/234,942 on 2019 Mar. 19. U.S. patent application Ser. No. 15/963,061 was a continuation-in-part of U.S. patent application Ser. No. 15/464,349 filed on 2017 Mar. 21 which issued as U.S. Pat. No. 9,968,297 on 2018 May 15. U.S. patent application Ser. No. 15/464,349 claimed the priority benefit of U.S. provisional patent application 62/430,667 filed on 2016 Dec. 6. U.S. patent application Ser. No. 15/464,349 was a continuation-in-part of U.S. patent application Ser. No. 15/136,948 filed on 2016 Apr. 24 which issued as U.S. patent Ser. No. 10/234,942 on 2019 Mar. 19. U.S. patent application Ser. No. 15/464,349 was a continuation-in-part of U.S. patent application Ser. No. 14/599,522 filed on 2015 Jan. 18 which issued as U.S. Pat. No. 9,814,426 on 2017 Nov. 14. U.S. patent application Ser. No. 15/464,349 was a continuation-in-part of U.S. patent application Ser. No. 14/562,719 filed on 2014 Dec. 7 which issued as U.S. patent Ser. No. 10/130,277 on 2018 Nov. 20. U.S. patent application Ser. No. 15/464,349 was a continuation-in-part of U.S. patent application Ser. No. 14/330,649 filed on 2014 Jul. 14.

U.S. patent application Ser. No. 15/236,401 was a continuation-in-part of U.S. patent application Ser. No. 15/136,948 filed on 2016 Apr. 24 which issued as U.S. patent Ser. No. 10/234,942 on 2019 Mar. 19. U.S. patent application Ser. No. 15/236,401 was a continuation-in-part of U.S. patent application Ser. No. 14/599,522 filed on 2015 Jan. 18 which issued as U.S. Pat. No. 9,814,426 on 2017 Nov. 14. U.S. patent application Ser. No. 15/136,948 claimed the priority benefit of U.S. provisional patent application 62/322,594 filed on 2016 Apr. 14. U.S. patent application Ser. No. 15/136,948 claimed the priority benefit of U.S. provisional patent application 62/303,126 filed on 2016 Mar. 3. U.S. patent application Ser. No. 15/136,948 claimed the priority benefit of U.S. provisional patent application 62/169,661 filed on 2015 Jun. 2. U.S. patent application Ser. No. 15/136,948 claimed the priority benefit of U.S. provisional patent application 62/160,172 filed on 2015 May 12. U.S. patent application Ser. No. 15/136,948 was a continuation-in-part of U.S. patent application Ser. No. 14/599,522 filed on 2015 Jan. 18 which issued as U.S. Pat. No. 9,814,426 on 2017 Nov. 14.

U.S. patent application Ser. No. 14/599,522 claimed the priority benefit of U.S. provisional patent application 62/089,696 filed on 2014 Dec. 9. U.S. patent application Ser. No. 14/599,522 claimed the priority benefit of U.S. provisional patent application 62/017,615 filed on 2014 Jun. 26. U.S. patent application Ser. No. 14/599,522 claimed the priority benefit of U.S. provisional patent application 61/939,244 filed on 2014 Feb. 12. U.S. patent application Ser. No. 14/599,522 claimed the priority benefit of U.S. provisional patent application 61/932,517 filed on 2014 Jan. 28. U.S. patent application Ser. No. 14/599,522 was a continuation-in-part of U.S. patent application Ser. No. 14/562,719 filed on 2014 Dec. 7 which issued as U.S. patent Ser. No. 10/130,277 on 2018 Nov. 20. U.S. patent application Ser. No. 14/562,719 claimed the priority benefit of U.S. provisional patent application 61/932,517 filed on 2014 Jan. 28. U.S. patent application Ser. No. 14/330,649 was a continuation-in-part of U.S. patent application Ser. No. 13/797,955 filed on 2013 Mar. 12 which issued as U.S. Pat. No. 9,456,916 on 2016 Oct. 4. U.S. patent application Ser. No. 14/330,649 was a continuation-in-part of U.S. patent application Ser. No. 13/523,739 filed on 2012 Jun. 14 which issued as U.S. Pat. No. 9,042,596 on 2015 May 26. U.S. patent application Ser. No. 13/797,955 claimed the priority benefit of U.S. provisional patent application 61/729,494 filed on 2012 Nov. 23.

The entire contents of these applications are incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to wearable mobile EEG devices.

### Introduction

EEG glasses are eyeglasses that incorporate electroencephalographic (EEG) sensors for sensing electromagnetic brain activity. EEG glasses have significant potential applications. For example, EEG eyeglasses can enable real-time monitoring, and ideally prediction, of seizures (such as epileptic seizures). Providing a person with advance notice of a pending seizure can greatly improve the quality of the person's life by enabling them to prepare for (or even mitigate) the seizure. EEG glasses can also be used as a Brain to Computer Interface (BCI) in situations where other forms of human-to-computer interaction (such as a touch-based interface or a speech-based interface) are undesirable, difficult, or even impossible. For example, EEG eyeglasses can serve as a method of communication by people who are paralyzed or have otherwise lost muscle control. In less dramatic but also useful applications, EEG eyeglasses can also serve as a BCI in situations where touch-based interaction is challenging (such as VR or AR applications) and/or speech-based interaction is inappropriate (such as during a public event when a person is expected to be silent). There are also numerous potential applications for EEG eyeglasses in other fields such as communication, sports, fitness, defense, fine arts, and entertainment.

One challenge in designing EEG eyeglasses (eyeglasses which incorporate electroencephalographic sensors) is how to place the EEG sensors in contact with a person's head in a manner which enables good electromagnetic contact with the body, but is also relatively unobtrusive visually. This can be particularly challenging for types of sensors for which hair between a sensor and a person's skin reduces electromagnetic contact. For example, a person's forehead may be a good location for electromagnetic contact, but it can be visually awkward for a person to wear eyeglasses with a component which spans across a person's forehead throughout daily life unless one is an engineer on a star ship. This application addresses this challenge with eyeglass designs which enable EEG sensors to come into good electromagnetic contact with a person's head without being too obtrusive visually.

Another challenge in designing EEG eyeglasses (eyeglasses which incorporate electroencephalographic sensors) is how to make them electromagnetically conductive, but also make them soft and compressive so that they conform to the surface of a person's head and are not uncomfortable. Many metals are good conductors, but are not soft or compressive. Many polymers are soft and compressive, but are not good conductors. This application addresses this challenge by specifying how to make EEG sensors with conductive, soft, and compressive materials such as soft polymers which are made conductive by impregnating, doping, and/or coating them with conductive material. In an example, an EEG sensor can be made with polydimethylsiloxane (PDMS) which has been impregnated, doped, or coated with carbon or silver particles or structures.

### Review of the Relevant Art

U.S. patent application 20200375524 (Aminifar et al, Dec. 3, 2020, “A Wearable System for Real-Time Detection of Epileptic Seizures”) by the École polytechnique fédérale de Lausanne discloses an innovative wearable system for epileptic seizure detection. This system comprises an eyeglasses frame with a left arm and a right arm configured to rest over the ears of an intended person wearing the eyeglasses, a first pair of electrodes located in the left arm, and a second pair of electrodes located in the right arm. The École polytechnique fédérale de Lausanne is pioneer in the development of mobile EEG monitoring for seizure detection.

U.S. patent application 20170188947 (Connor, Jul. 6, 2017, “EEG Glasses [Electroencephalographic Eyewear]”) and U.S. Pat. No. 9,968,297 (Connor, May 15, 2018, “EEG Glasses [Electroencephalographic Eyewear]”) disclose EEG glasses (electroencephalographic eyewear) with a side section of an eyewear frame which spans forward and upward onto a portion of a person's forehead and then curves back downward to connect to the front section of the eyewear frame.

U.S. patent applications 20110298706 (Mann, Dec. 8, 2011, “Brainwave Actuated Apparatus”) and 20170135597 (Mann, May 18, 2017, “Brainwave Actuated Apparatus”), a creative genius, disclose a brainwave actuated apparatus with a brainwave sensor for outputting a brainwave signal, an effector responsive to an input signal, and a controller operatively connected to an output of said brainwave sensor and a control input to said effector. U.S. patent application 20140223462 (Aimone et al., Aug. 7, 2014, “System and Method for Enhancing Content Using Brain-State Data”) discloses a computer system or method for modulating content based on a person's brainwave data, including modifying presentation of digital content at least one computing device. U.S. patent application 20140347265 (Aimone et al., Nov. 27, 2014, “Wearable Computing Apparatus and Method”) discloses a method performed by a wearable computing device comprising at least one bio-signal measuring sensor, the at least one bio-signal measuring sensor including at least one brainwave sensor. U.S. patent application 20160367189 (Aimone et al., Dec. 22, 2016, “Wearable Apparatus for Brain Sensors”) discloses a wearable apparatus with an outer band member comprising outer band ends joined by a curved outer band portion of a curve generally shaped to correspond to a user's forehead.

U.S. patent application 20190200925 (Aimone et al., Jul. 4, 2019, “Wearable Computing Device”) discloses a wearable device to wear on a head of a user including a flexible band generally shaped to correspond to the user's head, the band having at least a front portion to contact at least part of a frontal region of the user's head, a rear portion to contact at least part of an occipital region of the user's head, and at least one side portion extending between the front portion and the rear portion to contact at least part of an auricular region of the user's head. U.S. patent application 20200367789 (Moffat et al., Nov. 26, 2020, “Wearable Computing Apparatus with Movement Sensors and Methods Therefor”) discloses a wearable system for determining at least one movement property. U.S. patent Ser. No. 10/856,032 (Aimone et al., Dec. 1, 2020, “System and Method for Enhancing Content Using Brain-State Data”) discloses a computer system or method for modulating content based on a person's brainwave data, including modifying presentation of digital content at least one computing device.

U.S. patent application 20140316230 (Denison et al., Oct. 23, 2014, “Methods and Devices for Brain Activity Monitoring Supporting Mental State Development and Training”) discloses a method for receiving electroencephalography (EEG) data related to a user. U.S. patent application 20190101977 (Armstrong-Muntner et al., Apr. 4, 2019, “Monitoring a User of a Head-Wearable Electronic Device”) and U.S. patent Ser. No. 10/809,796 (Armstrong-Muntner et al., Oct. 20, 2020, “Monitoring a User of a Head-Wearable Electronic Device”) disclose an eye frame, a right light-emitting component, a left light-emitting component, and a processor to analyze light data indicative of the sensed right light and the sensed left light and determine a head gesture of the user based on the analyzed light data. U.S. patent application 20200060571 (Dauguet et al., Feb. 27, 2020, “Device for Measuring and/or Stimulating Brain Activity”) discloses an EEG device with means for transmitting and/or detecting physiological signals produced by the brain of an individual, and a support for the transmission and/or detection means, wherein the support is configured to extend over the top of the individual's head.

U.S. patent application 20160256086 (Byrd et al., Sep. 8, 2016, “Non-Invasive, Bioelectric Lifestyle Management Device”) discloses techniques to ascertain a biological condition such as blood glucose level, a heart rate, a blood ketone level, a blood alcohol content, a hydration level, a blood albumin level, and/or a blood electrolyte level. U.S. Pat. No. 9,204,796 (Tran, Dec. 8, 2015, “Personal Emergency Response (PER) System”) discloses one or more sensors to detect activities of a mobile object and a processor coupled to the sensor and the wireless transceiver to classify sequences of motions into groups of similar postures each represented by a model and to apply the models to identify an activity of the object. U.S. patent application 20160287173 (Abreu, Oct. 6, 2016, “Apparatus Configured to Support a Device on a Head”) discloses apparatuses support by at least a portion of a forehead in combination with at least one of a nose, an ear, and a head, or present an adjustable apparatus to provide improved fit of a head-positioned apparatus. U.S. patent applications 20060252978 (Vesely et al., Nov. 9, 2006, “Biofeedback Eyewear System”) and 20060252979 (Vesely et al., Nov. 9, 2006, “Biofeedback Eyewear System”) disclose a biofeedback eyewear system comprising stereo lenses, binaural audio and plurality of electrodes for biofeedback devices.

U.S. patent Ser. No. 10/860,097 (Chae, Dec. 8, 2020, “Eye-Brain Interface (EBI) System and Method for Controlling Same”) discloses methods and systems for calibrating an eye-brain interface (EBI) system controlled on the basis of eye movements and brain waves. U.S. patent application 20140023999 (Greder, Jan. 23, 2014, “Detection and Feedback of Information Associated with Executive Function”) discloses a neurosensing and feedback device to detect mental states and alert a wearer, such as in real-time. U.S. patent application 20170258410 (Gras, Sep. 14, 2017, “Method and Apparatus for Prediction of Epileptic Seizures”) discloses a system for predicting epileptic seizures including sensors operable to record a wearer's brain activity. U.S. patent application 20130242262 (Lewis, Sep. 19, 2013, “Enhanced Optical and Perceptual Digital Eyewear”) discloses wearable optics with a frame member, a lens, and circuitry within the frame member for enhancing the use of the wearable optics.

U.S. patent application 20070019279 (Goodall et al., Jan. 25, 2007, “Adjustable Lens System with Neural-Based Control”) discloses methods and systems for modifying or enhancing vision via analysis of neural or neuromuscular activity. U.S. patent application 20180204276 (Tumey, Jul. 19, 2018, “Brain Actuated Control of an E-Commerce Application”) discloses a brain-to-computer interface providing brain actuated control of a 3D virtual/augmented/mixed reality e-commerce application, effected by releasably attaching a plurality of high-impedance dry silver-based electrodes to selected locations on a human user's scalp. U.S. patent Ser. No. 10/867,720 (Mallires et al, Dec. 15, 2020, “Impregnation of a Non-Conductive Material with an Intrinsically Conductive Polymer”) discloses composite materials made by impregnating a non-conductive material with a conducting monomer to form a monomer-impregnated non-conductive material.

U.S. patent application 20070106172 (Abreu, May 10, 2007, “Apparatus and Method for Measuring Biologic Parameters”) discloses support structures for positioning sensors on a physiologic tunnel for measuring physical, chemical and biological parameters of the body and to produce an action according to the measured value of the parameters. U.S. patent application 20130056010 (Walker et al., Mar. 7, 2013, “Autonomous Positive Airway Pressure System”) discloses an apparatus, such as in the form of eyeglasses or goggles comprising dual lenses configured to serve as a gas chamber, or as headgear where the chamber is contoured to fit on the user's head and includes all components required to operate APAP, or the device may be configured to be placed on other locations such as the arm, torso, back, or leg.

In addition to the above patent literature, some academic researchers have done excellent work in the field of eyewear for mobile EEG monitoring. For example, Professor Kosmyna of MIT and her co-authors documented their prototyping work in (Kosmyna et al., 2019), “AttentivU: A Wearable Pair of EEG and EOG Glasses for Real-Time Physiological Processing,” 2019 IEEE 16th International Conference on Wearable and Implantable Body Sensor Networks (BSN), 19-22 May 2019. This article discloses eyeglasses which use both EEG and EOG for real-time monitoring of physiological data.

Also, Dr. Athanasios Vourvopoulos of the Instituto Superior Técnico published (Vourvopoulos et al., 2019), “EEGlass: An EEG-Eyeware Prototype for Ubiquitous Brain-Computer Interaction,” Adjunct Proceedings of the 2019 ACM International Joint Conference on Pervasive and Ubiquitous Computing (UbiComp), Conference Paper, September, 2019. This article discloses an EEG eyewear prototype comprised of plastic eyewear frames equipped with an Open-BCI board and a set of EEG electrodes at the contact points with the skull for unobtrusively collecting data concerning brain activity.

Further, (Abiri et al., 2019), “A Comprehensive Review of EEG-Based Brain—Computer Interface Paradigms,” Journal of Neural Engineering, February, 2019, 16(1), Epub Nov. 15, 2018, reviews EEG-based BCI paradigms including their advantages and disadvantages from a variety of perspectives. Also, (Bleihner et al., 2019), “Concealed, Unobtrusive Ear-Centered EEG Acquisition: cEEGrids for Transparent EEG,” Frontiers in Human Neuroscience, Apr. 7, 2017, 11, 163, discloses miniature electrodes placed in and around the human ear. Also, (Moses et al., 2019), “Real-Time Decoding of Question-and-Answer Speech Dialogue Using Human Cortical Activity,” Nature Communications, 2019, 10, 3096, discloses using high-density electrocorticography (ECoG) recordings to detect when subjects heard or said utterances and to then decode utterance identity. Also, (Tseghai et al., 2019), “The Status of Textile-Based Dry EEG Electrodes,” Autex Research Journal, 2020, reviews textile-based EEG sensors, including evaluation of their flexibility, stickability, and washability.

### SUMMARY OF THE INVENTION

Disclosed herein are innovative designs for smart eyeglasses and eyeglass accessories which enable mobile EEG monitoring while being relatively-unobtrusive for use during daily life. EEG eyeglasses or EEG-monitoring eyeglass accessories can enable monitoring (and ideally prediction) of epileptic seizures in real time. EEG eyeglasses or EEG-monitoring eyeglass accessories can also serve as a brain-to-computer interface in situations where touch-based interaction is challenging (such as VR or AR applications) and/or when speech-based interaction is inappropriate (such as during an event when a user is expected to be silent).

In an example, an eyeglass accessory device can comprise a band with EEG sensors which attaches to a person's eyeglasses with a clip, snap, clamp, hook, or plug and loops across the person's forehead, over the person's head, or around the back of the person's head. This enables a person to turn conventional prescription eyeglasses into part of an EEG monitoring system. Alternatively, a band with EEG sensors can be an integral part of smart eyeglasses (EEG eyeglasses) rather than an eyeglass accessory. In an example, a band with EEG sensors can be transparent. In an example, an EEG sensor can be made from polydimethylsiloxane, polybutylene terephthalate, or polyurethane which has been impregnated, doped, filled, and/or coated with silver, carbon nanotubes or other forms of carbon, copper, aluminum, nickel, platinum, or gold.

### INTRODUCTION TO THE FIGURES

FIG. 1 shows eyeglasses with an EEG sensor on a central third of a longitudinal sidepiece and an EEG sensor on a rear third of the longitudinal sidepiece.

FIG. 2 shows eyeglasses with an EEG sensor on an upwardly-convex portion of a longitudinal sidepiece.

FIG. 3 shows eyeglasses with an EEG sensor on a soft pad on an upwardly-convex portion of a longitudinal sidepiece.

FIG. 4 shows eyeglasses with an EEG sensor on a transparent soft pad on an upwardly-convex portion of a longitudinal sidepiece.

FIG. 5 shows eyeglasses with an EEG sensor on an upward arm or prong on a longitudinal sidepiece.

FIG. 6 shows eyeglasses with an EEG sensor on a transparent upward arm or prong on a longitudinal sidepiece.

FIG. 7 shows eyeglasses with an EEG sensor on a convex portion which encircles an ear.

FIG. 8 shows eyeglasses with an EEG sensor on a transparent convex portion which encircles an ear.

FIG. 9 shows eyeglasses with an EEG sensor on a band across a person's forehead.

FIG. 10 shows eyeglasses with an EEG sensor on a transparent band across a person's forehead.

FIG. 11 shows eyeglasses with an EEG sensor on a loop over the top of a person's head.

FIG. 12 shows eyeglasses with an EEG sensor on a transparent loop over the top of a person's head.

FIG. 13 shows eyeglasses with an EEG sensor on a loop around the back of a person's head.

FIG. 14 shows eyeglasses with an EEG sensor on a transparent loop around the back of a person's head.

FIG. 15 shows eyeglasses with an EEG sensor on a longitudinal sidepiece whose upper perimeter has an upward undulation onto a person's forehead.

FIG. 16 shows eyeglasses with an EEG sensor on a longitudinal sidepiece which bifurcates into an upper band and a lower band.

FIG. 17 shows an EEG sensor on an ear-circling ring which connects to a longitudinal sidepiece of eyeglasses.

FIG. 18 shows an EEG sensor on a forehead band which connects to a longitudinal sidepiece of eyeglasses.

FIG. 19 shows an EEG sensor on an upper loop which connects to a longitudinal sidepiece of eyeglasses.

FIG. 20 shows an EEG sensor on a rear loop which connects to a longitudinal sidepiece of eyeglasses.

FIG. 21 shows an EEG sensor on oblong soft pad which connects to a longitudinal sidepiece of eyeglasses.

FIG. 22 shows an EEG sensor on a crescent shaped electronics housing which connects to eyeglasses and is worn behind an ear.

FIG. 23 shows an EEG sensor on a circular or elliptical pad which connects to an upwardly-convex portion of a longitudinal sidepiece of eyeglasses.

FIG. 24 shows an EEG sensor on an upward arm or prong which connects to a longitudinal sidepiece of eyeglasses.

FIG. 25 shows an EEG sensor on a pivoting upward arm or prong on a longitudinal sidepiece of eyeglasses.

FIG. 26 shows an EEG sensor on an ear bud connected to a longitudinal sidepiece of eyeglasses.

FIG. 27 shows eyeglasses with an EEG sensor on a nose bridge and an EEG sensor on a longitudinal sidepiece.

FIG. 28 shows eyeglasses with an EEG sensor on a nose bridge and an EEG sensor on an upward-facing convexity of a longitudinal sidepiece.

FIG. 29 shows an EEG sensor on arcuate headband which connects to eyeglasses.

FIG. 30 shows eyeglasses with an EEG sensor on a longitudinal sidepiece which bifurcates into inner and outer branches.

FIG. 31 shows eyeglasses with an EEG sensor on a longitudinal sidepiece which bifurcates into upper and lower branches.

FIG. 32 shows eyeglasses with an EEG sensor on a longitudinal sidepiece which bifurcates into upper and lower branches and a rear loop around the back of a person's head.

FIG. 33 shows eyeglasses with an EEG sensor on a longitudinal sidepiece with an upward-facing convexity and a rear loop around the back of a person's head.

FIG. 34 shows an EEG sensor on an arcuate soft pad which is attached to a longitudinal sidepiece of eyeglasses.

FIG. 35 shows an EEG sensor on an ear-circling ring which is attached to a longitudinal sidepiece of eyeglasses.

FIG. 36 shows an EEG sensor on an upper loop or band (over the top of a person's head) which is attached to a longitudinal sidepiece of eyeglasses.

FIG. 37 shows an EEG sensor on a rear loop or band (around the back of a person's head) which is attached to a longitudinal sidepiece of eyeglasses.

FIG. 38 shows an EEG sensor on a wing-shaped soft pad which is attached to a longitudinal sidepiece of eyeglasses.

FIG. 39 shows eyeglasses with an EEG sensor on a movable arm which is pushed against a person's head by a spring.

FIG. 40 shows eyeglasses with an EEG sensor on a soft foam wedge.

FIG. 41 shows a first example of eyeglasses with an EEG sensor on a sinusoidal longitudinal sidepiece.

FIG. 42 shows eyeglasses with an EEG sensor on a longitudinal sidepiece whose upper perimeter has an upward-facing convexity and whose lower perimeter is substantially straight.

FIG. 43 shows eyeglasses with a longitudinal sidepiece whose rear portion has three arms.

FIG. 44 shows eyeglasses with a longitudinal sidepiece with an elliptical, circular, or oval shaped opening.

FIG. 45 shows eyeglasses with an EEG sensor on a sinusoidal longitudinal sidepiece.

FIG. 46 shows eyeglasses with an EEG sensor on a longitudinal sidepiece with a middle upward arch.

FIG. 47 shows eyeglasses with an EEG sensor on soft pad on a sinusoidal longitudinal sidepiece.

FIG. 48 shows eyeglasses with an EEG sensor on a soft pad on a longitudinal sidepiece with an middle upward arch.

FIG. 49 shows a first example of a headband with optical lenses and EEG sensors.

FIG. 50 shows a second example of a headband with optical lenses and EEG sensors.

FIG. 51 shows eyeglasses with an EEG sensor on a longitudinal sidepiece with a rear upward arch.

FIG. 52 shows eyeglasses with an EEG sensor on a longitudinal sidepiece with a shark-fin-shaped undulation.

FIG. 53 shows eyeglasses with an EEG sensor on a longitudinal sidepiece with arches over eyebrows.

FIG. 54 shows eyeglasses with an EEG sensor on a rear loop with upper and lower arm extensions.

FIG. 55 shows an EEG sensor on multi-lobed device which encircles a person's ear.

FIG. 56 shows eyeglasses with EEG sensors on upper and lower arms extending from a longitudinal sidepiece and on upper and lower arms extending from a rear loop.

FIG. 57 shows eyeglasses with EEG sensors on a transparent forehead loop and on a rear loop.

FIG. 58 shows eyeglasses with an EEG sensor on a head loop which moves (pivots) between a front configuration and a rear configuration.

FIG. 59 shows a first example of a headband with EEG sensors which connects to eyeglasses.

FIG. 60 shows a second example of a headband with EEG sensors which connects to eyeglasses.

FIG. 60 shows a neurostimulation device with an eyewear frame with a sidepiece whose mid-section curves upward.

### DETAILED DESCRIPTION OF THE FIGURES

FIG. 1 shows an oblique left-side view of an example of a mobile wearable EEG device (e.g. electroencephalographic glasses) comprising: right and left optical lenses, 101 and 103, which are configured to be worn in front of a person's right and left eyes, respectively; a bridge 102 which connects the right and left optical lenses; a longitudinal sidepiece (also called a “temple” on glasses) 104 which is attached to one of the lenses, extending from that lens to an ear and curving at least partially around the rear of the ear; a first electromagnetic energy sensor 105 which is located on a central third of the front-to-rear length of the longitudinal sidepiece; and a second electromagnetic energy sensor 106 which is located on a rear third of the front-to-rear length of the longitudinal sidepiece.

In an example, this device can have right and left side symmetry, with a corresponding longitudinal sidepiece and electromagnetic energy sensors on the other side of the person's head which is not shown in this figure. In an example, a longitudinal sidepiece and a bridge can be portions of the same continuous eyewear frame. In an example, the central third of the longitudinal sidepiece can curve and/or bend inward toward the surface of a person's head. In an example, an electromagnetic energy sensor can be gently pushed toward the surface of a person's head by a mechanism selected from the group consisting of: compressible foam or gel; a spring; a flexible prong; an inflatable chamber; a hydraulic piston; a rotatable threaded member; an electromagnetic solenoid; and a magnet. In example, the force and/or pressure with which an electromagnetic energy sensor is pushed toward a person's head can be adjusted by this mechanism. In an example, this device can include additional electromagnetic energy sensors along the longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 2 shows an oblique left-side view of an example of a mobile wearable EEG device (e.g. electroencephalographic glasses) comprising: right and left optical lenses, 201 and 203, which are configured to be worn in front of a person's right and left eyes, respectively; a bridge 202 which connects the right and left optical lenses; a longitudinal sidepiece (also called a “temple” on glasses) which further comprises a front portion 204 which is attached to one of the lenses, an upwardly-convex middle portion 205 which extends up onto the person's forehead, and a rear portion 207 which curves at least partially around the rear of the person's ear; a first electromagnetic energy sensor 206 on the upwardly-convex middle portion of the longitudinal sidepiece; and a second electromagnetic energy sensor 208 on the rear portion of the longitudinal sidepiece.

In an example, an upwardly-convex middle portion of a longitudinal sidepiece can be arcuate. In an example, an upwardly-convex middle portion can have a shape like a phase of a sinusoidal curve. In an example, an upwardly-convex middle portion can have a conic section shape. In an example, an upwardly-convex middle portion can comprise between one-quarter and one-half of the length of the longitudinal sidepiece. In an example, an upwardly-convex middle portion can curve and/or bend inward toward the surface of a person's head as well as curving upward onto the person's forehead. In an example, a peak of an upwardly-convex middle portion can be ¼″ to 3″ higher than the front portion (when a person's head is held upright). In an example, a peak of an upwardly-convex middle portion can be ½″ to 2″ higher than the front portion (when a person's head is held upright). In an example, a peak of an upwardly-convex middle portion can be ¼″ to 3″ higher than the top of a person's ear (when the person's head is held upright). In an example, a peak of an upwardly-convex middle portion can be ½″ to 2″ higher than the top of a person's ear (when the person's head is held upright).

In an example, this device can have right and left side symmetry, with a corresponding longitudinal sidepiece and electromagnetic energy sensors on the other side of the person's head which is not shown in this figure. In an example, a longitudinal sidepiece and a bridge can be portions of the same continuous eyewear frame. In an example, an electromagnetic energy sensor can be gently pushed toward the surface of a person's head by a mechanism selected from the group consisting of: compressible foam or gel; a spring; a flexible prong; an inflatable chamber; a hydraulic piston; a rotatable threaded member; an electromagnetic solenoid; and a magnet. In example, the force and/or pressure with which an electromagnetic energy sensor is pushed toward a person's head can be adjusted by this mechanism. In an example, this device can include additional electromagnetic energy sensors along the longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 3 shows an oblique left-side view of an example of a mobile wearable EEG device (e.g. electroencephalographic glasses) comprising: right and left optical lenses, 301 and 303, which are configured to be worn in front of a person's right and left eyes, respectively; a bridge 302 which connects the right and left optical lenses; a longitudinal sidepiece (also called a “temple” on glasses) which further comprises a front portion 304 which is attached to one of the lenses, an upwardly-convex middle portion 305 which extends up onto the person's forehead, and a rear portion 308 which curves at least partially around the rear of the person's ear; a soft arcuate pad 307 between the upwardly-convex middle portion and the surface of the person's head; a first electromagnetic energy sensor 306 on the soft arcuate pad; and a second electromagnetic energy sensor 309 on the rear portion of the longitudinal sidepiece.

In an example, an upwardly-convex middle portion of a longitudinal sidepiece can have a sinusoidal or conic section shape. In an example, an upwardly-convex middle portion can comprise between one-quarter and one-half of the length of the longitudinal sidepiece. In an example, an upwardly-convex middle portion can curve and/or bend inward toward the surface of a person's head as well as curving upward onto the person's forehead. In an example, a peak of an upwardly-convex middle portion can be ¼″ to 3″ higher or ½″ to 2″ higher than the front portion (when a person's head is held upright). In an example, a peak of an upwardly-convex middle portion can be ¼″ to 3″ higher or ½″ to 2″ higher than the top of the person's ear (when the person's head is held upright).

In an example, this device can have right and left side symmetry, with a corresponding longitudinal sidepiece and electromagnetic energy sensors on the other side of the person's head which is not shown in this figure. In an example, a longitudinal sidepiece and a bridge can be portions of the same continuous eyewear frame. In an example, a soft arcuate pad can be circular or elliptical. In an example, a soft arcuate pad can be made with compressible foam or gel. In an example, the soft arcuate pad in its uncompressed configuration can be from ⅛″ to 1″ thick. In an example, this device can include additional electromagnetic energy sensors along the longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 4 shows a device like the one in FIG. 3 except that the arcuate pad is at least partially transparent. FIG. 4 shows an oblique left-side view of an example of a mobile wearable EEG device (e.g. electroencephalographic glasses) comprising: right and left optical lenses, 401 and 403, which are configured to be worn in front of a person's right and left eyes, respectively; a bridge 402 which connects the right and left optical lenses; a longitudinal sidepiece (also called a “temple” on glasses) which further comprises a front portion 404 which is attached to one of the lenses, an upwardly-convex middle portion 405 which extends up onto the person's forehead, and a rear portion 408 which curves at least partially around the rear of the person's ear; a partially-transparent arcuate pad 407 between the upwardly-convex middle portion and the surface of the person's head; a first electromagnetic energy sensor 406 on the soft arcuate pad; and a second electromagnetic energy sensor 409 on the rear portion of the longitudinal sidepiece. Example variations and components discussed elsewhere in this disclosure and in priority-linked disclosures can also be applied to this example where relevant.

FIG. 5 shows an oblique left-side view of an example of a mobile wearable EEG device (e.g. electroencephalographic glasses) comprising: right and left optical lenses, 501 and 503, which are configured to be worn in front of a person's right and left eyes, respectively; a bridge 502 which connects the right and left optical lenses; a longitudinal sidepiece 504 (also called a “temple” on glasses) which extends from one of the lenses to an ear and curves at least partially around the rear of the ear; an arcuate arm or prong 505 which extends upward from the longitudinal sidepiece onto the person's forehead; and an electromagnetic energy sensor 506 on the arcuate arm or prong.

In an example, this device can have right and left side symmetry, with a corresponding longitudinal sidepiece and an electromagnetic energy sensor on the other side of the person's head which is not shown in this figure. In an example, a longitudinal sidepiece and a bridge can be portions of the same continuous eyewear frame. In an example, an arcuate arm or prong can have a forward-facing convexity. In an example, an arcuate arm or prong can bend and/or curve inward toward the surface of the person's head in addition to extending upward over the person's forehead. In an example, an electromagnetic energy sensor can be on the upper third of an arcuate arm or prong. In an example, this device can include additional electromagnetic energy sensors along a longitudinal sidepiece. In an example, this device can further comprise a battery, a data processor, a wireless data transmitter, and a wireless data receiver.

In an example, an arcuate arm or prong can be attached to a longitudinal sidepiece in a fixed configuration. In an example, an arcuate arm or prong can be movably attached to a longitudinal sidepiece. In an example, an arcuate arm or prong can be pivoted, rotated, or slid relative to a longitudinal sidepiece. In an example, an arcuate arm or prong can pivot, rotate, and/or slide from a first configuration in which it is substantially hidden behind the longitudinal sidepiece to a second configuration in which it extends upward from the longitudinal sidepiece onto a person's forehead.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 6 shows a device like the one in FIG. 5 except that the arcuate pad is at least partially transparent. FIG. 6 shows an oblique left-side view of an example of a mobile wearable EEG device (e.g. electroencephalographic glasses) comprising: right and left optical lenses, 601 and 603, which are configured to be worn in front of a person's right and left eyes, respectively; a bridge 602 which connects the right and left optical lenses; a longitudinal sidepiece 604 (also called a “temple” on glasses) which extends from one of the lenses to the person's ear and curves at least partially around the rear of the person's ear; a partially-transparent arcuate arm or prong 605 which extends upward from the longitudinal sidepiece onto the person's forehead; and an electromagnetic energy sensor 606 on the arcuate arm or prong. Example variations and components discussed elsewhere in this disclosure and in priority-linked disclosures can also be applied to this example where relevant.

FIG. 7 shows an oblique left-side view of an example of a mobile wearable EEG device (e.g. electroencephalographic glasses) comprising: right and left optical lenses, 701 and 703, which are configured to be worn in front of a person's right and left eyes, respectively; a bridge 702 which connects the right and left optical lenses; a longitudinal sidepiece (also called a “temple” on glasses) which further comprises a front portion 704 attached to one of the optical lenses, a middle portion 705 between the front portion and an ear, and a convex rear portion 707 which encircles the ear; a first electromagnetic energy sensor 706 on the middle portion of the longitudinal sidepiece; and a second electromagnetic energy sensor 708 on the convex rear portion of the longitudinal sidepiece.

In an example, this device can have right and left side symmetry, with a corresponding longitudinal sidepiece and electromagnetic energy sensors on the other side of the person's head which is not shown in this figure. In an example, a longitudinal sidepiece and a bridge can be portions of the same continuous eyewear frame. In an example, a convex rear portion can be elliptical, egg-shaped, or circular. In an example, a convex rear portion can be pear-shaped or teardrop-shaped. In an example, a convex rear portion can be an integral part of a longitudinal sidepiece. In an example, a convex rear portion can be attached to a longitudinal sidepiece. In an example, this device can include additional electromagnetic energy sensors along a longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 8 shows a device like the one in FIG. 7 except that the convex rear portion of the longitudinal sidepiece is at least partially transparent. FIG. 8 shows an oblique left-side view of an example of a mobile wearable EEG device (e.g. electroencephalographic glasses) comprising: right and left optical lenses, 801 and 803, which are configured to be worn in front of a person's right and left eyes, respectively; a bridge 802 which connects the right and left optical lenses; a longitudinal sidepiece (also called a “temple” on glasses) which further comprises a front portion 804 attached to one of the optical lenses, a middle portion 805 between the front portion and the person's ear, and a partially-transparent convex rear portion 807 which encircles an ear; a first electromagnetic energy sensor 806 on the middle portion of the longitudinal sidepiece; and a second electromagnetic energy sensor 808 on the convex rear portion of the longitudinal sidepiece. Example variations and components discussed elsewhere in this disclosure and in priority-linked disclosures can also be applied to this example where relevant.

FIG. 9 shows an oblique left-side view of an example of a mobile wearable EEG device (e.g. electroencephalographic glasses) comprising: right and left optical lenses, 901 and 903, which are configured to be worn in front of a person's right and left eyes, respectively; a bridge 902 which connects the right and left optical lenses; a longitudinal sidepiece 904 (also called a “temple” on glasses) which extends from one of the optical lenses to an ear and also curves at least partially around the rear of the ear; a forehead band (or strap) 905 which spans across the person's forehead from the longitudinal sidepiece; and an electromagnetic energy sensor 906 on the forehead band (or strap).

In an example, a longitudinal sidepiece and a bridge can be portions of the same continuous eyewear frame. In an example, this device can have right and left side symmetry, with a corresponding longitudinal sidepiece and electromagnetic energy sensor on the other side of the person's head which is not shown in this figure. In an example, the forehead band (or strap) can connect to the longitudinal sidepiece on the other side of the person's head. In an example, a forehead band can be connected to the middle third of the length of the longitudinal sidepiece. In an example, the connection of the forehead band with the longitudinal sidepiece can form a forward-facing acute angle in the range of 20 to 60 degrees. In an example, a forehead band can be stretchable and/or elastic. In an example, this angle can be adjusted. In an example, a forehead band can span the central third portion (measured vertically) of the person's forehead. In an example, there can be two (e.g. right side and left side) electromagnetic sensors on a forehead band. In an example, there can be three (e.g. right side, central, and left side) electromagnetic sensors on a forehead band. In an example, this device can include additional electromagnetic energy sensors along a longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 10 shows a device like the one in FIG. 9 except that the forehead band (or strap) is at least partially transparent. FIG. 10 shows an oblique left-side view of an example of a mobile wearable EEG device (e.g. electroencephalographic glasses) comprising: right and left optical lenses, 1001 and 1003, which are configured to be worn in front of a person's right and left eyes, respectively; a bridge 1002 which connects the right and left optical lenses; a longitudinal sidepiece 1004 (also called a “temple” on glasses) which extends from one of the optical lenses to an ear and also curves at least partially around the rear of the ear; a partially-transparent forehead band (or strap) 1005 which spans across the person's forehead from the longitudinal sidepiece; and an electromagnetic energy sensor 1006 on the forehead band (or strap). Example variations and components discussed elsewhere in this disclosure and in priority-linked disclosures can also be applied to this example where relevant.

FIG. 11 shows an oblique left-side view of an example of a mobile wearable EEG device (e.g. electroencephalographic glasses) comprising: right and left optical lenses, 1101 and 1103, which are configured to be worn in front of a person's right and left eyes, respectively; a bridge 1102 which connects the right and left optical lenses; a longitudinal sidepiece 1104 (also called a “temple” on glasses) which extends from one of the optical lenses to an ear and also curves at least partially around the rear of the ear; an upper loop (e.g. loop, strap, or band) 1105 which loops over the top of the person's head from the longitudinal sidepiece; and an electromagnetic energy sensor 1106 on the forehead band (or strap).

In an example, a longitudinal sidepiece and a bridge can be portions of the same continuous eyewear frame. In an example, this device can have right and left side symmetry, with a corresponding longitudinal sidepiece and electromagnetic energy sensor on the other side of the person's head which is not shown in this figure. In an example, an upper loop (e.g. loop, strap, or band) can connect to a longitudinal sidepiece on the other side of the person's head. In an example, an upper loop (e.g. loop, strap, or band) can loop over a person's head from a left-side longitudinal sidepiece to a right-side longitudinal sidepiece. In an example, an upper loop can be stretchable and/or elastic. In an example, the angle at which the upper loop connects to the longitudinal sidepiece can be adjusted. In an example, an upper loop can have an undulating shape. In an example, an upper loop can have a forward-facing concavity.

In an example, an electromagnetic energy sensor on an upper loop can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on an upper loop. In an example, there can be three (e.g. right side, central, and left side) electromagnetic sensors on an upper loop. In an example, this device can include additional electromagnetic energy sensors along a longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 12 shows a device like the one in FIG. 11 except that the upper loop (e.g. loop, strap, or band) is at least partially transparent. FIG. 12 shows an oblique left-side view of an example of a mobile wearable EEG device (e.g. electroencephalographic glasses) comprising: right and left optical lenses, 1201 and 1203, which are configured to be worn in front of a person's right and left eyes, respectively; a bridge 1202 which connects the right and left optical lenses; a longitudinal sidepiece 1204 (also called a “temple” on glasses) which extends from one of the optical lenses to an ear and also curves at least partially around the rear of the ear; a partially-transparent upper loop (e.g. loop, strap, or band) 1205 which loops over the top of the person's head from the longitudinal sidepiece; and an electromagnetic energy sensor 1206 on the forehead band (or strap). Example variations and components discussed elsewhere in this disclosure and in priority-linked disclosures can also be applied to this example where relevant.

FIG. 13 shows an oblique left-side view of an example of a mobile wearable EEG device (e.g. electroencephalographic glasses) comprising: right and left optical lenses, 1301 and 1303, which are configured to be worn in front of a person's right and left eyes, respectively; a bridge 1302 which connects the right and left optical lenses; a longitudinal sidepiece 1304 (also called a “temple” on glasses) which extends from one of the optical lenses to an ear and also curves at least partially around the rear of the ear; a rear loop (e.g. loop, strap, or band) 1306 which loops around the back of the person's head from the longitudinal sidepiece; a first electromagnetic energy sensor 1305 on the longitudinal sidepiece; and a second electromagnetic energy sensor 1307 on the rear loop.

In an example, a longitudinal sidepiece and a bridge can be portions of the same continuous eyewear frame. In an example, this device can have right and left side symmetry, with a corresponding longitudinal sidepiece and electromagnetic energy sensor on the other side of the person's head which is not shown in this figure. In an example, a rear loop (e.g. loop, strap, or band) can connect to a longitudinal sidepiece on the other side of the person's head. In an example, a rear loop (e.g. loop, strap, or band) can loop around the rear of a person's head from a left-side longitudinal sidepiece to a right-side longitudinal sidepiece. In an example, a rear loop can have an undulating shape. In an example, a rear loop can have an upward-facing concavity.

In an example, an electromagnetic energy sensor on a rear loop can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on a rear loop. In an example, there can be three (e.g. right side, central, and left side) electromagnetic sensors on a rear loop. In an example, this device can include additional electromagnetic energy sensors along a longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 14 shows a device like the one in FIG. 13 except that the upper loop (e.g. loop, strap, or band) is at least partially transparent. FIG. 14 shows an oblique left-side view of an example of a mobile wearable EEG device (e.g. electroencephalographic glasses) comprising: right and left optical lenses, 1401 and 1403, which are configured to be worn in front of a person's right and left eyes, respectively; a bridge 1402 which connects the right and left optical lenses; a longitudinal sidepiece 1404 (also called a “temple” on glasses) which extends from one of the optical lenses to an ear and also curves at least partially around the rear of the ear; a partially-transparent rear loop (e.g. loop, strap, or band) 1406 which loops around the back of the person's head from the longitudinal sidepiece; a first electromagnetic energy sensor 1405 on the longitudinal sidepiece; and a second electromagnetic energy sensor 1407 on the rear loop. Example variations and components discussed elsewhere in this disclosure and in priority-linked disclosures can also be applied to this example where relevant.

FIG. 15 shows an oblique left-side view of an example of a mobile wearable EEG device (e.g. electroencephalographic glasses) comprising: a longitudinal sidepiece 1501 (also called a “temple” on glasses) which extends from an optical lens to an ear, wherein an upper perimeter of a middle section (e.g. the middle third of the length) of the longitudinal sidepiece has an upward undulation which extends upward onto the side of the person's forehead; and an electromagnetic energy sensor 1502 on the middle section of longitudinal sidepiece.

In an example, an upward undulation of the upper perimeter of a middle section of a longitudinal sidepiece can have an upward-facing convexity. In an example, an upward undulation of the upper perimeter of a middle section of a longitudinal sidepiece can have a shape which is a portion of a sinusoidal wave. In an example, an upward undulation of the upper perimeter of a middle section of a longitudinal sidepiece can be skewed forward. In an example, the lower perimeter of the middle section of the longitudinal sidepiece can be substantially straight. In an example, the lower perimeter of the middle section of the longitudinal sidepiece can have a downward undulation. In an example, the average vertical width of the middle third of (the length of) a longitudinal sidepiece can be at least twice the average vertical width of the rear third of the longitudinal sidepiece. In an example, the maximum vertical width of the middle third of (the length of) a longitudinal sidepiece can be at least twice the maximum vertical width of the rear third of the longitudinal sidepiece.

In an example, this device can have right and left side symmetry, with a corresponding longitudinal sidepiece and electromagnetic energy sensor on the other side of the person's head which is not shown in this figure. In an example, this device can include additional electromagnetic energy sensors along a longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 16 shows an oblique left-side view of an example of a mobile wearable EEG device (e.g. electroencephalographic glasses) comprising: a longitudinal sidepiece (also called a “temple” on glasses) which extends from an optical lens to an ear, wherein an upper perimeter of a middle portion of the longitudinal sidepiece bifurcates into an upper band 1602 and a lower band 1603, and wherein the upper band and the lower band merge into the front and rear portions of the longitudinal sidepiece; and an electromagnetic energy sensor 1601 on the upper band.

In an example, an upper band can extend upward over the side of a person's forehead. In an example, an upper band can have an upward-facing convexity. In an example, an upper band can have a shape which is a portion of a sinusoidal wave. In an example, an upper band can have an upward-facing convexity which is skewed forward. In an example, a lower band can be substantially straight. In an example, a lower band can have a downward undulation. In an example, there can be a gap or opening between an upper band and the lower band. In an example, the gap or opening between an upper band and a lower band can have a swoosh, minnow, or teardrop shape.

In an example, this device can have right and left side symmetry, with a corresponding longitudinal sidepiece and electromagnetic energy sensor on the other side of the person's head which is not shown in this figure. In an example, this device can include additional electromagnetic energy sensors along a longitudinal sidepiece. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 17 shows an oblique left-side view of an example of a mobile wearable EEG device comprising: an ear-circling ring 1702 which encircles a person's ear; at least one clip, snap, clamp, hook, or plug 1701 which attaches the ear-circling ring to an eyewear frame; and at least one electromagnetic energy sensor 1703 on the ear-circling ring. In an example, an ear-circling ring can be at least partially transparent. In an example, an ear-circling ring can be circular, elliptical, or oval. In an example, an ear-circling ring can be elliptical or oval, with a longitudinal axis with a forward-tilted lower end. In an example, an ear-circling ring can be elliptical or oval with a longitudinal axis which has been rotated clockwise between 20 and 60 degrees. In an example, an ear-circling ring can have a pear shape. In an example, an ear-circling ring can be a ring with a continuous circumference.

In an example, an ear-circling ring can have a circumferential gap. In an example, an ear-circling ring can clip, snap, clamp, hook, or plug onto an eyewear frame at two or more places. Alternatively, an ear-circling ring can be attached to an eyewear frame by adhesive or Velcro™. Since the ear-circling ring can be removably connected to different eyewear frames, this design has the advantage of allowing a person to use this device with their current prescription eyeglasses, rather than having to buy a new frame and lenses. Conventional eyeglasses can become part of a mobile EEG monitoring system. In an example, an ear-circling ring can be closer to the surface of the person's head than an eyewear frame. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 18 shows an oblique left-side view of an example of a mobile wearable EEG device comprising: a forehead band (or strap) 1802 which spans across a person's forehead; at least one clip, snap, clamp, hook, or plug 1803 which connects the forehead band to an eyewear frame; and at least one electromagnetic energy sensor 1801 on the forehead band. In an example, the forehead band (or strap) can also connect to the eyewear frame on the other side of the person's head. In an example, the forehead band (or strap) can be (at least partially) transparent. In an example, a forehead band can be connected to the middle third of the length of the eyewear frame. Since the forehead band can be removably connected to different eyewear frames, this design has the advantage of allowing a person to use this device with their current prescription eyeglasses, rather than having to buy a new frame and lenses. Conventional eyeglasses can become part of a mobile EEG monitoring system.

In an example, the connection of a forehead band with a eyewear frame can form a forward-facing acute angle in the range of 20 to 60 degrees. In an example, this angle can be adjusted. In an example, a forehead band can be stretchable and/or elastic. In an example, a forehead band can span the central third portion (measured vertically) of the person's forehead. In an example, there can be two (e.g. right side and left side) electromagnetic sensors on a forehead band. In an example, there can be three (e.g. right side, central, and left side) electromagnetic sensors on a forehead band.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. In an example, this device can further comprise a battery, a data processor, a wireless data transmitter, and a wireless data receiver.

In an example, a mobile wearable EEG device can comprise: a forehead band which is configured to span across a person's forehead; at least one clip, snap, clamp, hook, or plug which connects the forehead band to a longitudinal sidepiece of an eyewear frame; and at least one electromagnetic energy sensor on the forehead band. In an example, the forehead band can be transparent. In an example, the forehead band can be connected to the middle third of the length of the longitudinal sidepiece of the eyewear frame. In an example, the connection of a forehead band with the longitudinal sidepiece of the eyewear frame can form a forward-facing acute angle in the range of 20 to 60 degrees. In an example, this device can further comprise a battery, a data processor, a wireless data transmitter, and a wireless data receiver. Example variations and components discussed elsewhere in this disclosure and in priority-linked disclosures can also be applied to this example where relevant.

FIG. 19 shows an oblique left-side view of an example of a mobile wearable EEG device comprising: an upper loop (e.g. loop, strap, or band) 1901 which loops over the top of a person's head; at least one clip, snap, clamp, hook, or plug 1903 which connects the upper loop to an eyewear frame; and at least one electromagnetic energy sensor 1902 on the upper loop. In an example, the upper loop (e.g. loop, strap, or band) can also connect to an eyewear frame on the other side of the person's head. In an example, an upper loop can loop over a person's head from the left-side of an eyewear frame to the right-side of the eyewear frame. In an example, an upper loop can be (at least partially) transparent. In an example, an upper loop can be connected (via snap or clip) to the middle third of the length of an eyewear frame. Since the upper loop can be removably connected to different eyewear frames, this design has the advantage of allowing a person to use this device with their current prescription eyeglasses, rather than having to buy a new frame and lenses. Conventional eyeglasses can become part of a mobile EEG monitoring system.

In an example, the angle at which an upper loop connects to an eyewear frame can be adjusted. In an example, an upper loop can be stretchable and/or elastic. In an example, an upper loop can have an undulating shape. In an example, an upper loop can have a forward-facing concavity. In an example, an electromagnetic energy sensor on an upper loop can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on an upper loop. In an example, there can be three (e.g. right side, central, and left side) electromagnetic sensors on an upper loop. In an example, this device can further comprise a battery, a data processor, a wireless data transmitter, and a wireless data receiver.

In an example, there can be two (e.g. right side and left side) electromagnetic sensors on an upper loop. In an example, there can be three (e.g. right side, central, and left side) electromagnetic sensors on an upper loop. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices.

In an example, a mobile wearable EEG device can comprise: an upper loop which is configured to loop over the top of a person's head; at least one clip, snap, clamp, hook, or plug which connects the upper loop to a longitudinal sidepiece of an eyewear frame; and at least one electromagnetic energy sensor on the upper loop. In an example, the upper loop can be transparent. In an example, the upper loop can be connected to a middle third of the length of the longitudinal sidepiece of the eyewear frame. In an example, the upper loop can have an undulating shape. In an example, the upper loop can have a forward-facing concavity. In an example, at least one electromagnetic energy sensor on the upper loop can have protrusions which protrude between strands of hair in order to be in contact the person's scalp and/or be in better electromagnetic communication with the person's brain. In an example, hair-penetrating protrusions can be made from polydimethylsiloxane, polybutylene terephthalate, or polyurethane which has been impregnated, doped, filled, and/or coated with silver, carbon nanotubes or other forms of carbon, copper, aluminum, nickel, platinum, or gold. Example variations and components discussed elsewhere in this disclosure and in priority-linked disclosures can also be applied to this example where relevant.

FIG. 20 shows an oblique left-side view of an example of a mobile wearable EEG device comprising: a rear loop (e.g. loop, strap, or band) 2002 which loops around the rear of a person's head; at least one clip, snap, clamp, hook, or plug 2001 which connects the rear loop to an eyewear frame; and at least one electromagnetic energy sensor 2003 on the rear loop. In an example, a rear loop (e.g. loop, strap, or band) can also connect to an eyewear frame on the other side of the person's head. In an example, a rear loop can loop around the rear of the person's head from a left-side of an eyewear frame to a right-side of the eyewear frame. In an example, a rear loop can be (at least partially) transparent. In an example, a rear loop can be stretchable and/or elastic. In an example, a rear loop can have an undulating shape. In an example, a rear loop can have an upward-facing concavity. Since the rear loop can be removably connected to different eyewear frames, this design has the advantage of allowing a person to use this device with their current prescription eyeglasses, rather than having to buy a new frame and lenses. Conventional eyeglasses can become part of a mobile EEG monitoring system.

In an example, an electromagnetic energy sensor on a rear loop can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on a rear loop. In an example, there can be three (e.g. right side, central, and left side) electromagnetic sensors on a rear loop. In an example, this device can further comprise a battery, a data processor, a wireless data transmitter, and a wireless data receiver.

In an example, there can be two (e.g. right side and left side) electromagnetic sensors on a rear loop. In an example, there can be three (e.g. right side, central, and left side) electromagnetic sensors on a rear loop. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 21 shows an oblique left-side view of an example of a mobile wearable EEG device comprising: an oblong soft pad 2101; at least one clip, snap, clamp, hook, or plug 2103 which connects the oblong soft pad to an eyewear frame; and at least one electromagnetic energy sensor 2102 on the oblong soft pad. In an example, an oblong soft pad can be (at least partially) transparent. In an example, an oblong soft pad can have a rounded rectangular shape (e.g. a rectangle with rounded vertexes). In an example, an oblong soft pad can have an elliptical or oval shape. In an example, an oblong soft pad can have a pear or peanut shape. In an example, an oblong soft pad can have a crescent or boomerang shape. In an example, a soft pad can be sinusoidal or have a carlavian curve perimeter.

In an example, an oblong soft pad can be attached (e.g. by clips or snaps) at two locations to an eyewear frame. Alternatively, an oblong soft pad can be attached to an eyewear frame by adhesive or Velcro™. Since the oblong soft pad can be removably connected to different eyewear frames, this design has the advantage of allowing a person to use this device with their current prescription eyeglasses, rather than having to buy a new frame and lenses. Conventional eyeglasses can become part of a mobile EEG monitoring system. In an example, the length of an oblong soft pad can between 1″ and 4″. In an example, the front half of an oblong soft pad can be thicker than the rear half of an oblong soft pad. In an example, the front half of an oblong soft pad can more than twice as thick as the rear half of an oblong soft pad. In an example, there can be two or more electromagnetic energy sensors along the oblong soft pad.

In an example, an oblong soft pad can comprise compressible foam or gel. In an example, an oblong soft pad can be made from a soft (e.g. elastomeric) conductive polymer-based material. In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices.

In an example, a mobile wearable EEG device can comprise: an oblong soft pad; at least one clip, snap, clamp, hook, or plug which connects the oblong soft pad to a longitudinal sidepiece of an eyewear frame; and at least one electromagnetic energy sensor on the oblong soft pad. In an example, the oblong soft pad can be transparent. In an example, the oblong soft pad can have a rounded rectangular shape. In an example, the oblong soft pad can have an elliptical or oval shape. In an example, the oblong soft pad can have a crescent or boomerang shape. In an example, a front half of the oblong soft pad can be thicker than a rear half of the oblong soft pad. In an example, a front half of the oblong soft pad can be at least twice as thick as a rear half of the oblong soft pad. In an example, at least one electromagnetic energy sensor can be made from polydimethylsiloxane, polybutylene terephthalate, or polyurethane which has been impregnated, doped, filled, and/or coated with silver, carbon nanotubes or other forms of carbon, copper, aluminum, nickel, platinum, or gold. Example variations and components discussed elsewhere in this disclosure and in priority-linked disclosures can also be applied to this example where relevant.

FIG. 22 shows an oblique left-side view of an example of a mobile wearable EEG device comprising: a crescent (or “C” or boomerang) shaped electronics housing 2202 which is worn at least partially behind a person's ear; at least one clip, snap, clamp, hook, or plug 2201 which connects the crescent shaped electronics housing to an eyewear frame; and at least one electromagnetic energy sensor 2203 on the crescent shaped electronics housing. In an example, a crescent (or “C” or boomerang) shaped electronics housing can be (at least partially) transparent. In an example, a crescent shape electronics housing can have a rear-facing convexity. In an example, a crescent shape electronics housing can curve around a portion of the circumference of a person's ear from the top of the person's ear to their ear lobe. In an example, a crescent shaped electronics housing and/or an electromagnetic energy sensor can also be attached to a person's ear lobe.

In an example, a crescent shaped electronics housing can be attached (e.g. by clips or snaps) at two locations to an eyewear frame. Alternatively, a crescent shaped electronics housing can be attached to an eyewear frame by adhesive or Velcro™. Since the crescent shaped electronics housing can be removably connected to different eyewear frames, this design has the advantage of allowing a person to use this device with their current prescription eyeglasses, rather than having to buy a new frame and lenses. Conventional eyeglasses can become part of a mobile EEG monitoring system. In an example, there can be two or more electromagnetic energy sensors along a crescent shaped electronics housing. In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control (e.g. people with ALS). In an example, this device can function as a BCI for remote control of computers or other devices. Example variations and components discussed elsewhere in this disclosure and in priority-linked disclosures can also be applied to this example where relevant.

FIG. 23 shows an oblique left-side view of an example of a mobile wearable EEG system comprising: an eyeglasses sidepiece 2301 with an upwardly-convex middle portion which arcs upward to cover a portion of a person's forehead and/or temple, a convex (e.g. circular or elliptical) soft pad 2303; at least one clip, snap, clamp, hook, or plug 2304 which connects the convex soft pad to the upwardly-convex middle portion of the eyeglasses sidepiece; and at least one electromagnetic energy sensor 2302 on the convex soft pad. In an example, a convex soft pad can be at least partially transparent.

In an example, there can be two or more electromagnetic energy sensors on a convex soft pad. In an example, a convex soft pad can comprise compressible foam or gel. In an example, a convex soft pad can be made from a soft (e.g. elastomeric) conductive polymer-based material. In an alternative example, a convex soft pad can be attached to an eyewear frame by adhesive or Velcro™. Since the convex soft pad can be removably connected to different eyewear frames, this design has the advantage of allowing a person to use this device with their current prescription eyeglasses, rather than having to buy a new frame and lenses. Conventional eyeglasses can become part of a mobile EEG monitoring system.

In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this system can further comprise a battery, a data processor, a wireless data transmitter, and a wireless data receiver.

In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control, or for remote control of other systems. Example variations and components discussed elsewhere in this disclosure and in priority-linked disclosures can also be applied to this example where relevant.

FIG. 24 shows an oblique left-side view of an example of a mobile wearable EEG device comprising: an arcuate upward arm 2401 which extends upward from a sidepiece of eyeglasses to cover a portion of a person's forehead and/or temple; at least one clip, snap, clamp, hook, or plug 2403 which connects the arcuate upward arm to the sidepiece; and at least one electromagnetic energy sensor 2402 on the arcuate upward arm. In an example, the arcuate upward arm can be at least partially transparent. In an example, the arcuate upward arm can have a forward-facing convexity. In an example, an arcuate upward arm can have a boomerang or crescent shape. In an example, there can be two or more electromagnetic energy sensors on an arcuate upward arm.

In an example, an arcuate upward arm can further comprise compressible foam or gel. In an example, an arcuate upward arm can be made from a soft (e.g. elastomeric) conductive polymer-based material. In an alternative example, an arcuate upward arm can be attached to an eyewear frame by adhesive or Velcro™. Since the arcuate upward arm can be removably connected to different eyewear frames, this design has the advantage of allowing a person to use this device with their current prescription eyeglasses, rather than having to buy a new frame and lenses. Conventional eyeglasses can become part of a mobile EEG monitoring system. In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver.

In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 25 shows an oblique left-side view of an example of a mobile wearable EEG device (e.g. electroencephalographic glasses) comprising: a longitudinal sidepiece 2501 (also called a “temple” on glasses) which extends from an optical lens to a person's ear; a longitudinal pivoting arm 2503, wherein the longitudinal pivoting arm has a first configuration in which its longitudinal axis is substantially aligned with a longitudinal axis of the longitudinal sidepiece, and wherein the longitudinal pivoting arm has a second configuration in which it extends upward from the longitudinal sidepiece to cover a portion of the person's forehead; and wherein the longitudinal pivoting arm is pivoted (e.g. rotated) from its first configuration to its second configuration, or vice versa; and at least one electromagnetic energy sensor 2502 on the longitudinal pivoting arm. In an example, the longitudinal pivoting arm can be at least partially transparent.

In an example, a longitudinal pivoting arm can have an oblong shape. In an example, a longitudinal pivoting arm can pivot around a connection to an eyeglass sidepiece which is on the rear half (e.g. the rear end) of the longitudinal pivoting arm. In an example, a longitudinal pivoting arm can be substantially hidden behind an eyeglass sidepiece when the arm is in its first configuration. In an example, the intersection of a longitudinal pivoting arm and an eyeglass sidepiece can form an acute forward-facing angle (e.g. between 20 and 80 degrees) when the pivoting arm is in its second configuration. In an example, the intersection of the longitudinal axes of the pivoting arm and the eyeglass sidepiece can form an acute forward-facing angle when the pivoting arm is in its second configuration. In an example, the longitudinal axes of the pivoting arm and the eyeglass sidepiece can be orthogonal when the pivoting arm is in its second configuration.

In an example, there can be two or more electromagnetic energy sensors on a longitudinal pivoting arm. In an example, a longitudinal pivoting arm can be made from a soft (e.g. elastomeric) conductive polymer-based material. In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver.

In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 26 shows an oblique left-side view of an example of a mobile wearable EEG device (e.g. electroencephalographic glasses) comprising: a longitudinal sidepiece 2601 (also called a “temple” on glasses) which extends from an optical lens to a person's ear; a first electromagnetic energy sensor 2602 on the longitudinal sidepiece; an ear insert (e.g. ear bud) 2603 which is configured to be inserted into the person's ear, wherein the ear insert is connected to the longitudinal sidepiece by a flexible conductive wire 2605; and a second electromagnetic energy sensor 2604 on the ear insert. In an example, there can be two or more electromagnetic energy sensors on a longitudinal sidepiece. In an example, the flexible conductive wire can have a helical and/or undulating shape. In a variation on this example, an ear insert (e.g. ear bud) can be in wireless communication with a longitudinal sidepiece instead of being connected by a flexible conductive wire. In an example, an ear insert can be at least partially transparent.

In an example, an ear insert (e.g. ear bud) can be made from a soft (e.g. elastomeric) conductive polymer-based material.

In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 27 shows an oblique left-side view of an example of a mobile wearable EEG device (e.g. electroencephalographic glasses) comprising: a longitudinal sidepiece 2703 of a pair of eyeglasses, wherein the longitudinal sidepiece has a middle portion which extends from an optical lens to a person's ear and a rear portion which curves at least partially around the rear of the person's ear; a first electromagnetic energy sensor 2704 on the middle portion of the longitudinal sidepiece; a second electromagnetic energy sensor 2705 on the rear portion of the longitudinal sidepiece; a nose bridge 2701 which connects optical lenses of the eyeglasses; and a third electromagnetic energy sensor 2702 on the nose bridge (or the nose pads thereof).

In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 28 shows an oblique left-side view of an example of a mobile wearable EEG device (e.g. electroencephalographic glasses) comprising: a longitudinal sidepiece 2803 of a pair of eyeglasses, wherein the longitudinal sidepiece which extends from an optical lens to a person's ear, wherein the longitudinal sidepiece has a middle portion with an upward-facing convexity that extends upward onto the person's forehead, wherein the longitudinal sidepiece has a rear portion which curves at least partially around the rear of the person's ear; a first electromagnetic energy sensor 2804 on the middle portion of the longitudinal sidepiece; a second electromagnetic energy sensor 2805 on the rear portion of the longitudinal sidepiece; a nose bridge 2801 which connects optical lenses of the eyeglasses; and a third electromagnetic energy sensor 2802 on the nose bridge (or the nose pads thereof).

In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 29 shows an oblique left-side view of an example of a mobile wearable EEG system comprising: an arcuate headpiece (or headband) 2904 with a front portion which spans across a person's forehead and left and right rear portions which at least partially curve behind the person's left ear and right ears, respectively; clips, snaps, or clamps, including 2905, which connect the arcuate headpiece (or headband) to eyeglasses 2903; a first electromagnetic energy sensor 2901 on the front portion of the arcuate headpiece (or headband); a second electromagnetic energy sensor 2902 on the front portion of the arcuate headpiece (or headband); and a third electromagnetic energy sensor 2906 on a rear portion of the arcuate headpiece (or headband).

In an example, an arcuate headpiece (or headband) can be at least partially transparent. In an example, the front portion of an arcuate headpiece (or headband) can tilt upwards. In an example, the intersection of an arcuate headpiece (or headband) and a sidepiece (or “temple”) of eyeglasses can form a forward-facing acute angle between 10 and 60 degrees. In an example, a first electromagnetic energy sensor can be on the middle of the person's forehead and a second electromagnetic energy sensor can be on the side of the person's forehead. In an example, an arcuate headpiece (or headband) can be stretchable and/or elastic. In an example, an arcuate headpiece (or headband) can be porous and/or made with breathable material. In an example, a front portion of an arcuate headpiece (or headband) can span a person's forehead on the middle third (measured vertically from eyes to hairline) of the person's forehead.

In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 30 shows an oblique left-side view of an example of a mobile wearable EEG device (e.g. electroencephalographic glasses) comprising: eyeglasses with a longitudinal sidepiece (also called a “temple”) which spans from an optical lens to an ear; wherein a front portion of the longitudinal sidepiece bifurcates into an inner branch 3002 which is attached to the optical lens at a first location at a first distance from the person's nose and an outer branch 3003 which is attached to the optical lens at a second location at a second distance from the person's nose, and wherein the second distance is greater than the first distance; wherein a rear portion 3004 of the longitudinal sidepiece which curves at least partially around the rear of the person's ear; and an electromagnetic energy sensor 3001 on the inner branch of the front portion of the longitudinal sidepiece.

In an example, the inner branch can be closer to the surface of the person's head than the outer branch. In an example, the inner branch can curve closely around the contour of the person's temple and/or forehead to bring an electromagnetic energy sensor into close contact with the person's temple and/or forehead. In an example, the front portion of the longitudinal sidepiece can bifurcate in a rear-to-front direction. In an example, the inner branch can be arcuate and the outer branch can be straight. In an example, the bifurcated front portion of the longitudinal sidepiece can span the front third of the length of the longitudinal sidepiece. In an example, the bifurcated front portion of the longitudinal sidepiece can span the front half of the length of the longitudinal sidepiece. In an example, an inner branch can connect to the top of an optical lens and an outer branch can connect to the side of an optical lens. In an example, the inner branch can be at least partially transparent.

In an example, an inner branch of a longitudinal sidepiece can be soft and compressible. In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 31 shows an oblique left-side view of an example of a mobile wearable EEG device (e.g. electroencephalographic glasses) comprising: eyeglasses with an longitudinal sidepiece (also called a “temple”) which spans from an optical lens to an ear, wherein a middle portion of the longitudinal sidepiece splits into an arcuate upper branch 3102 and an arcuate lower branch 3101; and an electromagnetic energy sensor 3103 on the upper branch. In an example, an arcuate upper branch can extend up onto the person's forehead and/or temple. In an example, an arcuate upper branch can be at least partially transparent. In an example, an upper branch can have an upward-facing convexity and a lower branch can have a downward-facing convexity. In an example, an upper branch can have an upward undulation and a lower branch can have a downward undulation. In an example, an upper branch can have a shape which is one-half of first sine wave and a lower branch can have a shape which is one-half of a second sine wave, wherein the second sine wave which is 180 degrees out of phase with the first sine wave.

In an example, there is a gap (e.g. gap, opening, or hole) between an upper branch and a lower branch. In an example, this gap can be convex. In an example, this gap can be elliptical or oval. In an example, this gap can have a tear-drop, swoosh, or fish shape. In an example, a gap between an upper branch and a lower branch can have a stylized-eye shape. In an example, a gap between an upper branch and a lower branch can have an upper perimeter of the gap which is half of a first sine wave and a lower perimeter which is half of a second sine wave which is 180 degrees out of phase with the first since wave. In an example, an upper branch can be closer to the surface of the person's head than a lower branch. In an example, this gap can have a maximum vertical width in the range of ½″ to 4″. In an example, this gap can have a maximum vertical width in the range of 1″ to 3″. In an example, this gap can have a maximum horizontal length in the range of 1″ to 5″. In an example, this gap can have a maximum horizontal length in the range of 2″ to 4″.

In an example, an electromagnetic energy sensor can be made from a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 32 shows an oblique left-side view of an example of a mobile wearable EEG device (e.g. electroencephalographic glasses) comprising: eyeglasses with a longitudinal sidepiece (also called a “temple”) which spans from an optical lens to an ear, wherein a middle (or front) portion of the longitudinal sidepiece splits into an arcuate upper branch 3202 and an arcuate lower branch 3201; a first electromagnetic energy sensor 3203 on the upper branch; a rear loop 3205 which extends from the longitudinal sidepiece around the rear of a person's head; and a second electromagnetic energy sensor 3204 on the rear loop.

In an example, an arcuate upper branch can curve up onto a person's forehead and/or temple. In an example, an arcuate upper branch can be at least partially transparent. In an example, an upper branch can have an upward-facing convexity and a lower branch can have a downward-facing convexity. In an example, an upper branch can have an upward undulation and a lower branch can have a downward undulation. In an example, an upper branch can have a shape which is one-half of first sine wave and a lower branch can have a shape which is one-half of a second sine wave, wherein the second sine wave which is 180 degrees out of phase with the first sine wave.

In an example, there can be a gap (e.g. gap, opening, or hole) between an upper branch and a lower branch. In an example, this gap can be convex. In an example, this gap can have an elliptical or oval shape. In an example, this gap can have a tear-drop, swoosh, or fish shape. In an example, a gap between an upper branch and a lower branch can have a stylized-eye shape. In an example, a gap between an upper branch and a lower branch can have an upper perimeter which is half of a first sine wave and a lower perimeter which is half of a second sine wave, wherein the second sine wave is 180 degrees out of phase with the first since wave. In an example, an upper branch can be closer to the surface of the person's head than a lower branch. In an example, a gap can have a maximum vertical width in the range of ½″ to 4.″ In an example, a gap can have a maximum vertical width in the range of 1″ to 3.″ In an example, a gap can have a maximum horizontal length in the range of 1″ to 5.″ In an example, a gap can have a maximum horizontal length in the range of 2″ to 4.″

In an example, a rear loop can be stretchable and/or elastic. In an example, a rear loop can be at least partially transparent. In an example, a rear loop can curve around the rear of a person's head, from a right-side longitudinal side piece on the right side of the person's head to a left-side longitudinal side piece on the left side of the person's head. In an example, a rear loop can have a maximum height which is higher than the top of the person's ear. In an example, a rear loop can have a maximum height which is at least 1″ higher than the top of the person's ear. In an example, a rear loop can have an upward-facing convexity as is curves around the rear of a person's head. In an example, a rear loop can have a lowest height which is lower than the top of the person's ear. In an example, a rear loop can have a lowest height which is at least 1″ lower than the top of the person's ear. In an example, a rear loop can have a downward-facing convexity as is curves around the rear of a person's head.

In an example, an electromagnetic energy sensor on a rear loop (or on a housing on the rear loop) can have protrusions (e.g. prongs, teeth, or combs) which protrude between strands of hair in order to be in contact with the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, a soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on a rear loop. In an example, there can be three (e.g. right side, central, and left side) electromagnetic sensors on a rear loop.

In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 33 shows an oblique left-side view of an example of a mobile wearable EEG device (e.g. electroencephalographic glasses) comprising: eyeglasses with a longitudinal sidepiece (also called a “temple”) which spans from an optical lens to an ear, wherein a middle portion 3301 of the longitudinal sidepiece has an upward undulation (e.g. an upward-facing convexity) which curves up onto the person's forehead and/or temple; a first electromagnetic energy sensor 3302 on the middle portion of the longitudinal sidepiece; a rear loop 3304 which extends from the longitudinal sidepiece around the rear of a person's head; and a second electromagnetic energy sensor 3303 on the rear loop. In an example, an arcuate middle portion of a sidepiece can be at least partially transparent. In an example, a middle portion of a sidepiece can have a sinusoidal shape (e.g. at least a portion of a sinusoidal wave).

In an example, a rear loop can be stretchable and/or elastic. In an example, a rear loop can be at least partially transparent. In an example, a rear loop can curve around the rear of a person's head, from a right-side longitudinal side piece on the right side of the person's head to a left-side longitudinal side piece on the left side of the person's head. In an example, a rear loop can have a maximum height which is higher than the top of the person's ear. In an example, a rear loop can have a maximum height which is at least 1″ higher than the top of the person's ear. In an example, a rear loop can have an upward-facing convexity as is curves around the rear of a person's head. In an example, a rear loop can have a lowest height which is lower than the top of the person's ear. In an example, a rear loop can have a lowest height which is at least 1″ lower than the top of the person's ear. In an example, a rear loop can have a downward-facing convexity as is curves around the rear of a person's head.

In an example, an electromagnetic energy sensor on a rear loop (or on a housing on the rear loop) can have protrusions (e.g. prongs, teeth, or combs) which protrude between strands of hair in order to be in contact with the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, a soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on a rear loop. In an example, there can be three (e.g. right side, central, and left side) electromagnetic sensors on a rear loop.

In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 34 shows an oblique left-side view of an example of a mobile wearable EEG device comprising: an arcuate soft pad 3402 which is attached to a longitudinal sidepiece (also called a “temple”) of eyeglasses by at least one clip, clamp, snap, or plug 3404, wherein the arcuate soft pad extends upward from the longitudinal sidepiece to cover a portion of the person's forehead and/or temple; and a plurality of electromagnetic energy sensors 3401 and 3403 on the arcuate soft pad. Alternatively, an arcuate soft pad can be attached to an eyewear frame by adhesive or Velcro™.

In an example, an arcuate soft pad can be transparent or translucent. In an example, an arcuate soft pad can be convex. In an example, an arcuate soft pad can have a circular, elliptical, or oval shape. In an example, an arcuate soft pad can have an oblong or rounded-rectangular shape. In an example, an arcuate soft pad can have a “flat tire shape” (i.e. the shape of a circle whose lower perimeter has been compressed toward its center). In an example, an arcuate soft pad can be attached to a middle portion (e.g. the middle third) of a longitudinal sidepiece. In an example, an arcuate soft pad can be closer to the surface of the person's head than an eyewear frame. In an example, an arcuate soft pad can be attached to a longitudinal sidepiece at two locations. Since an arcuate soft pad can be removably connected to different eyewear frames, this design has the advantage of allowing a person to use this device with their current prescription eyeglasses, rather than having to buy a new frame and lenses. Conventional eyeglasses can become part of a mobile EEG monitoring system.

In an example, an arcuate soft pad can comprise a compressible and/or elastomeric polymer. In an example, an arcuate soft pad can comprise a silicone-based polymer (e.g. polydimethylsiloxane, polybutylene terephthalate, or polyurethane). In an example, an electromagnetic energy sensor can be made with a soft (e.g. elastomeric) conductive polymer-based material. In an example, a soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on an arcuate soft pad. In an example, there can be three (e.g. right side, central, and left side) electromagnetic sensors on an arcuate soft pad.

In an example, this device can further comprise a battery, a data processor, a wireless data transmitter, and a wireless data receiver. In an example, electromagnetic energy sensors can be EEG (electroencephalographic) sensors and/or electrodes which detect, measure, and/or record the electromagnetic activity of a person's brain. In an example, this electromagnetic activity can be analyzed in order to detect and/or predict (epileptic) seizures. In an example, this device can function as a BCI (Brain Computer Interface) for communication purposes, such as for people who have lost motor control, or for remote control of other devices. Example variations and components discussed elsewhere in this disclosure and in priority-linked disclosures can also be applied to this example where relevant.

FIG. 35 shows an oblique left-side view of an example of a mobile wearable EEG system comprising: an eyewear frame 3501; a partially-transparent ear-circling ring 3504 which encircles a person's ear; at least one clip, snap, clamp, hook, or plug 3502 which attaches the ear-circling ring to the eyewear frame; and a plurality of electromagnetic energy sensors 3503 and 3505 on the ear-circling ring. In an example, an ear-circling ring can have a circular, elliptical, or oval shape. In an example, an ear-circling ring can be elliptical or oval, with a longitudinal axis with a forward-tilted lower end. In an example, an ear-circling ring can be elliptical or oval with a longitudinal axis which has been rotated clockwise between 20 and 60 degrees. In an example, an ear-circling ring can have a pear shape. In an example, an ear-circling ring can be a ring with a continuous circumference. In an example, an ear-circling ring can have a circumferential gap.

In an example, an ear-circling ring can clip, snap, clamp, hook, or plug onto the sidepiece (e.g. the “temple”) of an eyewear frame. Alternatively, an ear-circling ring can be attached to an eyewear frame by adhesive or Velcro™. Since an ear-circling ring can be removably connected to different eyewear frames, this design has the advantage of allowing a person to use this device with their current prescription eyeglasses, rather than having to buy a new frame and lenses. Conventional eyeglasses can become part of a mobile EEG monitoring system. In an example, an ear-circling ring can be closer to the surface of the person's head than an eyewear frame. In an example, this device can further comprise a battery, a data processor, a wireless data transmitter, and a wireless data receiver.

In an example, an ear-circling ring can comprise a compressible and/or elastomeric polymer. In an example, an ear-circling ring can comprise a silicone-based polymer (e.g. polydimethylsiloxane, polybutylene terephthalate, or polyurethane). In an example, an electromagnetic energy sensor can be made with a soft (e.g. elastomeric) conductive polymer-based material. In an example, a soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 36 shows an oblique left-side view of an example of a mobile wearable EEG system comprising: an eyewear frame 3601; a partially-transparent upper loop (e.g. strap, band, or arm) 3602 which loops over the top of a person's head; at least one clip, snap, clamp, hook, or plug 3605 which attaches the upper loop to the eyewear frame; and a plurality of electromagnetic energy sensors 3603 and 3604 on the upper loop. In an example, an upper loop can clip, snap, clamp, hook, or plug onto the sidepiece (e.g. the “temple”) of an eyewear frame. Alternatively, an upper loop can be attached to an eyewear frame by adhesive or Velcro™. In an example, an upper loop can be stretchable and/or elastic. In an example, the forward-facing angle between the eyewear frame and the upper loops can be adjusted. In an example, this angle can be obtuse. Since an upper loop can be removably connected to different eyewear frames, this design has the advantage of allowing a person to use this device with their current prescription eyeglasses, rather than having to buy a new frame and lenses. Conventional eyeglasses can become part of a mobile EEG monitoring system.

In an example, an upper loop (or an electromagnetic energy sensor on an upper loop) can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on an upper loop. In an example, there can be three (e.g. right side, central, and left side) electromagnetic sensors on an upper loop. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 37 shows an oblique left-side view of an example of a mobile wearable EEG device comprising: a semi-circumferential arcuate band (e.g. band, arm, or strap) 3704 which attaches to a longitudinal sidepiece (e.g. “temple”) of an eyewear frame, wherein the semi-circumferential arcuate band curves around the rear of a person's head and spans between 50% and 80% of the circumference of a person's head in a substantially horizontal plane (e.g. a plane which is within 20 degrees of being horizontal); at least one clip, snap, clamp, hook, or plug (3702 and 3703) which attaches the semi-circumferential arcuate band to the longitudinal sidepiece of the eyewear frame; and a plurality of electromagnetic energy sensors 3701 and 3705 on the semi-circumferential arcuate band. Alternatively, a semi-circumferential arcuate band can be attached to an eyewear frame by adhesive or Velcro™. Since a semi-circumferential arcuate band can be removably connected to different eyewear frames, this design has the advantage of allowing a person to use this device with their current prescription eyeglasses, rather than having to buy a new frame and lenses. Conventional eyeglasses can become part of a mobile EEG monitoring system. In an example, a semi-circumferential arcuate band can be at least partially transparent. In an example, a semi-circumferential arcuate band can be stretchable and/or elastic.

In an example, a semi-circumferential arcuate band can clip, snap, clamp, hook, or plug onto a sidepiece (e.g. the “temple”) of an eyewear frame. In an example, a semi-circumferential arcuate band can clip, snap, clamp, hook, or plug onto a sidepiece of an eyewear frame at two or more locations. In an example, a front portion of a semi-circumferential arcuate band can be substantially parallel to a longitudinal sidepiece of an eyewear frame. In an example, a front portion of a semi-circumferential arcuate band which clips, snaps, clamps, or plugs onto a sidepiece of an eyewear frame can be substantially parallel to the sidepiece. In an example, a rear portion of a semi-circumferential arcuate band can have an upward bend. In an example, a rear portion of a semi-circumferential arcuate band can have an upward bend, but the overall best-fitting plane for the semi-circumferential arcuate band can still be within 20 degrees of horizontal when a person's head is held upright. In an example, a rear portion of a semi-circumferential arcuate band can have a downward bend. In an example, a rear portion of a semi-circumferential arcuate band can have a downward bend, but the overall best-fitting plane for the semi-circumferential arcuate band can still be within 20 degrees of horizontal when a person's head is held upright.

In an example, a semi-circumferential arcuate band (or an electromagnetic energy sensor on a semi-circumferential arcuate band) can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on a semi-circumferential arcuate band. In an example, there can be three (e.g. right side, central, and left side) electromagnetic sensors on a semi-circumferential arcuate band. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 38 shows an oblique left-side view of an example of a mobile wearable EEG device comprising: an arcuate soft pad 3802 with an upper wing and a lower wing, wherein the arcuate soft pad attaches to a longitudinal sidepiece (e.g. “temple”) of an eyewear frame, wherein the upper wing extends upward from the longitudinal sidepiece onto a side of a person's forehead, and wherein the lower wing extends downward from the longitudinal sidepiece toward the person's ear; at least one clip, snap, clamp, hook, or plug 3803 which attaches the arcuate soft pad to the longitudinal sidepiece of the eyewear frame; a first electromagnetic energy sensor 3801 on the upper wing of the arcuate soft pad; and a second electromagnetic energy sensor 3804 on the lower wing of the arcuate soft pad. Since an arcuate soft pad with upper and lower wings can be removably connected to different eyewear frames, this design has the advantage of allowing a person to use this device with their current prescription eyeglasses, rather than having to buy a new frame and lenses. In this way, conventional eyeglasses can become part of a mobile EEG monitoring system.

In an example, a soft pad with upper and lower wings can be at least partially transparent. In an example, a soft pad with upper and lower wings can have a forward-facing convexity. In an example, a soft pad with upper and lower wings can have a boomerang shape. In an example, a soft pad with upper and lower wings can have a peanut shape. In an example, an upper wing can extend upward between ½″ and 4″ above a longitudinal sidepiece. In an example, an upper wing can extend upward between 1″ and 3″ above a longitudinal sidepiece. In an example, a lower wing can extend downward between ½″ and 4″ below a longitudinal sidepiece. In an example, a lower wing can extend downward between 1″ and 3″ below a longitudinal sidepiece. In an example, a soft pad with upper and lower wings can be attached to an eyewear frame by adhesive or Velcro™ instead of a clip, snap, clamp, hook, or plug.

In an example, an arcuate soft pad with wings (or an electromagnetic energy sensor on an arcuate soft pad) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, there can be two (e.g. right side and left side) electromagnetic sensors on a semi-circumferential arcuate band. In an example, there can be three (e.g. right side, central, and left side) electromagnetic sensors on a semi-circumferential arcuate band. In an example, this device can further comprise a battery, a data processor, a wireless data transmitter, and a wireless data receiver.

In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 39 shows an oblique left-side view of an example of a mobile wearable EEG device (e.g. electroencephalographic glasses) comprising: a longitudinal sidepiece (e.g. sometimes called a “temple”) 3901 of an eyewear frame; a movable arm (e.g. arm, branch, pad, prong, or protrusion) 3904 which covers a portion of a person's forehead (or temple); a spring (e.g. spring or coil) 3902 between the longitudinal sidepiece and the moveable arm, wherein the spring is attached to the moveable arm and forces the moveable arm toward the person's forehead (or temple); and an electromagnetic energy sensor 3903 on the moveable arm.

In an example, a moveable arm can be (at least partially) transparent. In an example, one end of a moveable arm can be connected to a longitudinal sidepiece. In an example, one end of a moveable arm can be connected to a longitudinal sidepiece by a hinge or other moveable joint. In an example, a moveable arm can pivot or rotate relative to a longitudinal sidepiece. In an example, a moveable arm can have an oblong shape. In an example, a moveable arm can have an arcuate oblong shape. In an example, a moveable arm can have a boomerang or peanut shape. In an example, a moveable arm can extend inward (toward the surface of a person's head) and upward (onto a person's forehead) from a longitudinal sidepiece. In an example, a rear portion (e.g. end) of a moveable arm can be connected to a longitudinal sidepiece via a hinge or other moveable joint. In an example, a front portion (e.g. end) of a moveable arm can be connected to a longitudinal sidepiece via a hinge or other moveable joint. In an example, one end of a moveable arm can be connected to a longitudinal sidepiece via a hinge or other moveable joint and the other end of the moveable arm can be connected to the longitudinal sidepiece via a spring (or coil).

In an example, a moveable arm (or an electromagnetic energy sensor on a moveable arm) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 40 shows an oblique left-side view of an example of a mobile wearable EEG device (e.g. electroencephalographic glasses) comprising: a longitudinal sidepiece (e.g. sometimes called a “temple”) 4003 of an eyewear frame; a soft wedge 4001 between the longitudinal sidepiece and the person's forehead; and an electromagnetic energy sensor 4002 on the soft wedge. In an example, a soft wedge can be (at least partially) transparent. In an example, a soft wedge can comprise compressible foam. In an example, a soft wedge can be attached to (or part of) a longitudinal sidepiece. In an example, a front portion of a soft wedge can be wider than a rear portion of the soft wedge. In an example, the front half of a soft wedge can be wider than the rear half of the soft wedge. In an example, a front portion of a soft wedge can extend inward from a longitudinal sidepiece toward the surface of a person's forehead.

In an example, a front portion of a soft wedge can extend between ¼″ and 1″ inward from a longitudinal sidepiece toward the surface of a person's forehead. In an example, a front portion of a soft wedge can extend between ¼″ and 1″ inward from a longitudinal sidepiece toward the surface of a person's head and a rear portion of a soft wedge can extend between ⅛″ and ½″ inward from a longitudinal sidepiece toward the surface of a person's forehead. In an example, the front half of a soft wedge can extend between ¼″ and 1″ inward from a longitudinal sidepiece toward the surface of a person's head and a rear half of a soft wedge can extend between ⅛″ and ½″ inward from a longitudinal sidepiece toward the surface of a person's forehead.

In an example, a soft wedge (or an electromagnetic energy sensor on a soft wedge) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 41 shows an oblique left-side view of an example of a mobile wearable EEG device (e.g. electroencephalographic glasses) comprising: a longitudinal sidepiece (e.g. sometimes called a “temple”) of an eyewear frame, wherein the longitudinal sidepiece further comprises an arcuate front portion with an upward-facing convexity 4101, an arcuate rear portion with a downward-facing convexity 4103, and a substantially-straight portion 4104 between the arcuate front portion and the arcuate rear portion; a first electromagnetic energy sensor 4102 on the arcuate front portion; and a second electromagnetic energy sensor 4105 on the arcuate rear portion. In an example, the arcuate front and rear portions together can comprise a generally-sinusoidal wave shape or “S” shape. In an example, there can be gaps between front and rear portions and the substantially-straight portion. In an example, arcuate front and rear portions can be (at least partially) transparent. In an example, an arcuate front portion can extend upward (and inward) onto a portion of the person's forehead. In an example, an arcuate front portion can span the front half of a longitudinal sidepiece and an arcuate rear portion can span the rear half of the longitudinal sidepiece.

In an example, a front or rear arcuate portion (or an electromagnetic energy sensor on a front or rear arcuate portion) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 42 shows an oblique left-side view of an example of a mobile wearable EEG device (e.g. electroencephalographic glasses) comprising: a longitudinal sidepiece 4201 (e.g. sometimes called a “temple”) of an eyewear frame, wherein the longitudinal sidepiece further comprises a middle section 4203, wherein the middle section is wider (vertically) than front and rear sections of the longitudinal sidepiece, wherein an upper perimeter of the middle section has an upward-facing convexity, and wherein a lower perimeter of the middle section is substantially straight; and an electromagnetic energy sensor 4202 on the middle section. In an example, the middle section of the longitudinal sidepiece can extend upward (and inward) onto a portion of the person's forehead. In an example, the middle section can have a maximum (vertical) width in the range of ½″ to 2.″ In an example, the upper perimeter of the middle section can have a sinusoidal shape. In an example, the upper perimeter of the middle section can have a conic section shape. In an example, the upper perimeter of the middle section can have a semicircular shape. In an example, the upper perimeter of the middle section can be an arcuate curve with an upward-facing convexity which is skewed in a forward direction.

In an example, a middle section of a longitudinal sidepiece of an eyewear frame (or an electromagnetic energy sensor on a middle section) can be made with a soft (e.g. elastomeric) conductive polymer-based material. In an example, a conductive polymer-based material can be made by impregnating or coating polydimethylsiloxane with carbon or sliver. In an example, a conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can be made with hydrogel. In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 43 shows an oblique left-side view of an example of a mobile wearable EEG device (e.g. electroencephalographic glasses) comprising: a longitudinal sidepiece 4301 (e.g. sometimes called a “temple”) of an eyewear frame, wherein the longitudinal sidepiece spans from the front of the frame (e.g. from an optical lens) to a person's ear and curves around part of (the rear of) the person's ear; a first arcuate arm (e.g. arm, protrusion, or branch) 4303 which extends upward and forward from a location on the longitudinal sidepiece which is vertically-above the person's ear; a first electromagnetic energy sensor 4302 on the first arcuate arm; a second arcuate arm (e.g. arm, protrusion, or branch) 4305 which extends upward and rearward from a location on the longitudinal sidepiece which is vertically-above the person's ear; a second electromagnetic energy sensor 4304 on the second arcuate arm; and a third electromagnetic energy sensor 4307 on a portion 4306 of the longitudinal sidepiece which curves around part of (the rear of) the person's ear.

In an example, first and/or second arcuate arms (e.g. arms, protrusions, or branches) can be transparent. In an example, first and/or second arcuate arms can have a boomerang, crescent, semicircular, or peanut shape. In an example, first and second arcuate arms can combine to form a shape with an upward-facing concavity. In an example, first and second arcuate arms can combine to form a semicircular arc with an upward-facing concavity. In an example, first and second arcuate arms can be between 1″ and 3″ in length. In an example, first and second arcuate arms can be between 2″ and 4″ in length. In an example, first and second arcuate arms can extend upward from a location on a longitudinal sidepiece which is directly above a person's ear. In an example, first and second arcuate arms can extend between 1″ and 3″ higher than a person's ear (when a person's head is upright). In an example, first and second arcuate arms can extend between 2″ and 4″ higher than a person's ear (when a person's head is upright).

In an example, an arcuate arm (or an electromagnetic energy sensor on an arcuate arm) can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 44 shows an oblique left-side view of an example of a mobile wearable EEG device (e.g. electroencephalographic glasses) comprising: a longitudinal sidepiece 4401 (e.g. sometimes called a “temple”) of an eyewear frame, wherein the longitudinal sidepiece spans from the front of the frame (e.g. from an optical lens) to a person's ear, and wherein there is an elliptical, circular, or oval shaped opening (e.g. opening, hole, or gap) in a middle portion 4403 of the longitudinal sidepiece; and one or more electromagnetic energy sensors 4402 and 4404 on the middle portion of the longitudinal sidepiece. In an example, the middle portion can itself have an elliptical, circular, or oval shape. In an example, an upper arc of an elliptical, circular, or oval middle portion can extend upward onto a person's forehead. In an example, an elliptical, circular, or oval shaped opening in a longitudinal sidepiece can be between 1″ and 3″ in diameter. In an example, an elliptical, circular, or oval shaped opening in a longitudinal sidepiece can be between 2″ and 4″ in diameter.

In an example, a middle portion of a longitudinal sidepiece (or an electromagnetic energy sensor on a middle portion) can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 45 shows an oblique left-side view of an example of a mobile wearable EEG device (e.g. electroencephalographic glasses) comprising: a longitudinal sidepiece 4501 (e.g. sometimes called a “temple”) of an eyewear frame, wherein the longitudinal sidepiece spans from the front of the frame (e.g. from an optical lens) to a person's ear, and wherein a middle portion 4503 of the longitudinal sidepiece has a sinusoidal shape; and one or more electromagnetic energy sensors 4502 and 4504 on the middle portion of the longitudinal sidepiece. In an example, a wave of a sinusoidal middle portion can extend upward onto a person's forehead.

In an example, a middle portion of a longitudinal sidepiece (or an electromagnetic energy sensor on a middle portion) can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain.

In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 46 shows an oblique left-side view of an example of a mobile wearable EEG device (e.g. electroencephalographic glasses) comprising: a longitudinal sidepiece 4601 (e.g. sometimes called a “temple”) of an eyewear frame, wherein the longitudinal sidepiece spans from the front of the frame (e.g. from an optical lens) to a person's ear, and wherein a middle portion of the longitudinal sidepiece is an arch 4603 with an upward-facing convexity; and an electromagnetic energy sensor 4602 on the arch. In an example, an arch can extend upward onto a person's forehead. In an example, an arch can be a section of a circle or ellipse. In an example, an arch can have a semicircular or rainbow shape. In an example, an arch can have a conic section shape. In an example, an arch can have a parabolic shape.

In an example, an arch (or an electromagnetic energy sensor on an arch) can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 47 shows an oblique left-side view of an example of a mobile wearable EEG device (e.g. electroencephalographic glasses) comprising: a longitudinal sidepiece 4701 (e.g. sometimes called a “temple”) of an eyewear frame, wherein the longitudinal sidepiece spans from the front of the frame (e.g. from an optical lens) to a person's ear, and wherein a middle portion 4704 of the longitudinal sidepiece has a sinusoidal shape; a first soft pad 4703 between an upper wave of the sinusoidal middle portion and the surface of the person's head; a second soft pad 4706 between a lower wave of the sinusoidal middle portion and the surface of the person's head; a first electromagnetic energy sensor 4702 on the first soft pad; and a second electromagnetic energy sensor 4705 on the second soft pad. In an example, the first and/or second soft pads can be transparent.

In an example, a middle portion of a longitudinal sidepiece (or an electromagnetic energy sensor on a middle portion) can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 48 shows an oblique left-side view of an example of a mobile wearable EEG device (e.g. electroencephalographic glasses) comprising: a longitudinal sidepiece 4801 (e.g. sometimes called a “temple”) of an eyewear frame, wherein the longitudinal sidepiece spans from the front of the frame (e.g. from an optical lens) to a person's ear, and wherein a middle portion of the longitudinal sidepiece is an arch 4804 with an upward-facing convexity; a soft pad 4803 between the arch and the surface of the person's head; and an electromagnetic energy sensor 4802 on the arch. In an example, an arch can extend upward onto a person's forehead. In an example, an arch can be a section of a circle or ellipse. In an example, an arch can have a semicircular or rainbow shape. In an example, an arch can have a conic section shape. In an example, an arch can have a parabolic shape. In an example, the soft pad can be transparent.

In an example, an arch (or an electromagnetic energy sensor on an arch) can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 49 shows an oblique left-side view of an example of a mobile wearable EEG device (e.g. electroencephalographic glasses) comprising: a band 4903 which encircles a person's head in a substantially horizontal plane (e.g. within 10 degrees of horizontal); one or more optical lenses 4901 attached to the band; an arm 4905 which extends downward from the band and curves around at least part of a rear portion of the person's ear (e.g. curves behind the person's ear); a first electromagnetic energy sensor 4902 on a side (e.g. left or right side of the head) portion of the band; a second electromagnetic energy sensor 4904 on a rear (e.g. back of the head) portion of the band; and a third electromagnetic energy sensor 4906 on the arm. In an example, a band can have an elliptical or circular shape. In an example, a band can be continuous arcuate piece, which enables closer contact with the surface of a person's head than a traditional eyewear frame.

In an example, a front portion of a band can be resilient and/or stiff. In an example, a rear portion of a band can be stretchable and/or elastic. In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 50 shows an oblique left-side view of an example of a mobile wearable EEG device (e.g. electroencephalographic glasses) comprising: a band 5003 which encircles between 40% and 70% of the circumference of a person's head in a substantially horizontal plane (e.g. within 10 degrees of horizontal) and curves behind the person's ear; one or more optical lenses 5001 attached to the band; a first electromagnetic energy sensor 5002 on a portion of the band which spans the person's forehead; and a second electromagnetic energy sensor 5004 on a portion of the band behind the person's ear. In an example, a band can have a shape which is a segment of (e.g. between 40% and 70% of the circumference of) an ellipse or circle. In an example, a band can be continuous arcuate piece, which enables closer contact with the surface of a person's head than a traditional eyewear frame.

In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 51 shows an oblique left-side view of an example of a mobile wearable EEG device (e.g. electroencephalographic glasses) comprising: a longitudinal sidepiece 5101 (sometimes called a “temple”) of an eyewear frame, wherein a rear portion of the longitudinal sidepiece bifurcates, including an arch 5103 with an upward-facing convexity which is located vertically-above the person's ear, and wherein the rear portion of the longitudinal sidepiece also includes an arm 5104 which curves around at least a portion of the rear of the person's ear; a first electromagnetic energy sensor 5102 on the arch; and a second electromagnetic energy sensor 5105 on the arm.

In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 52 shows an oblique left-side view of an example of a mobile wearable EEG device (e.g. electroencephalographic glasses) comprising: a longitudinal sidepiece (sometimes called a “temple”) of an eyewear frame, wherein the longitudinal sidepiece bifurcates into an upper wave portion 5202 and a lower wave portion 5203 with a gap (e.g. gap, opening, or hole) between the upper wave portion and the lower wave portion, wherein the upper wave portion has an upward-and-forward-facing convexity which extends upward and forward onto the person's forehead, and wherein the lower wave portion has an upward-facing convexity; and an electromagnetic energy sensor 5201 on the upper wave portion. In an example, the upper wave portion can have a skewed sinusoidal shape (e.g. a shape like a portion of a sinusoidal wave which has been skewed and/or stretched in a forward direction). In an example, the gap between the upper and lower wave portions can have a rear-facing “shark fin” shape. In an example, the gap between the upper and lower wave portions can have a maximum height between 1″ and 3″. In an example, the gap between the upper and lower wave portions can have a maximum height between 2″ and 4″.

In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 53 shows an oblique left-side view of an example of a mobile wearable EEG device (e.g. electroencephalographic glasses) comprising: an eyewear frame, wherein the eyewear frame further comprises a longitudinal sidepiece 5303 (sometimes called a “temple”), wherein the eyewear frame also further comprises an arch 5301 with an upward-facing convexity on a person's forehead, wherein the arch is connected to the longitudinal sidepiece and to a bridge between optical lenses of the eyewear; and an electromagnetic energy sensor 5302 on the arch. In an example, an arch can be (at least partially) transparent. In an example, an arch can be above an eyebrow. In an example, there can be two arches (right and left), one above each eyebrow (right and left). In an example, an arch can be compelled toward contact with a person's forehead by a spring and/or elastic mechanism. In an example, an arch can have a partial-elliptical or partial-circular shape. In an example, an arch can have a sinusoidal phase shape. In an example, an arch can be substantially parallel with (but higher than) an eyebrow. In an example, an arch can extend between ½″ and 3″ above an optical lens.

In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 54 shows an oblique left-side view of an example of a mobile wearable EEG device (e.g. electroencephalographic glasses) comprising: an eyewear frame 5401 which holds optical lenses in front of a person's eyes and loops around the rear of the person's head; an upper arm 5403 (e.g. arm, branch, or protrusion) which extends upward from a portion of the eyewear frame which loops around the rear of the person's head; a lower arm 5404 (e.g. arm, branch, or protrusion) which extends downward from a portion of the eyewear frame which loops around the rear of the person's head; a first electromagnetic energy sensor 5402 on the upper arm; and a second electromagnetic energy sensor 5405 on the lower arm.

In an example, an upper and/or lower arm can be (at least partially) transparent. In an example, an upper and/or lower arm can be compelled toward contact with a person's head by a spring and/or elastic mechanism. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 55 shows an oblique left-side view of an example of a mobile wearable EEG device comprising: a wearable multi-lobed (or undulating) device which encircles a person's ear and is attached to an eyewear frame by one or more clips, clamps, snaps, or plugs 5503; wherein the multi-lobed device further comprises a front lobe (or undulation) 5502 in front of the person's ear and a first electromagnetic energy sensor 5501 on the front lobe (or undulation); wherein the multi-lobed device further comprises an upper lobe (or undulation) 5504 above the person's ear and a second electromagnetic energy sensor 5505 on the upper lobe (or undulation); and wherein the multi-lobed device further comprises a rear lobe (or undulation) 5506 behind the person's ear and a third electromagnetic energy sensor 5507 on the rear lobe (or undulation). In an example, a multi-lobed device can have four rounded vertexes (or undulations). In an example, a multi-lobed device can have a star shape. In an example, the multi-lobed device can have three rounded vertexes (or undulations).

In an example, a multi-lobed device can be (at least partially) transparent. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 56 shows an oblique left-side view of an example of a mobile wearable EEG device (e.g. electroencephalographic glasses) comprising: an eyewear frame 5601 which holds optical lenses in front of a person's eyes and loops around the rear of the person's head; a side upper arm 5603 (e.g. arm, branch, or protrusion) which extends upward from a portion of the eyewear frame which spans from an optical lens to an ear; a first electromagnetic energy sensor 5602 on side upper arm; a side lower arm 5608 (e.g. arm, branch, or protrusion) which extends downward from the portion of the eyewear frame which spans from an optical lens to an ear; and a second electromagnetic energy sensor 5609 on the side lower arm; a back upper arm 5605 (e.g. arm, branch, or protrusion) which extends upward from a portion of the eyewear frame which loops around the rear of the person's head; a third electromagnetic energy sensor 5604 on the back upper arm; a back lower arm 5606 (e.g. arm, branch, or protrusion) which extends downward from the portion of the eyewear frame which loops around the rear of the person's head; and a fourth electromagnetic energy sensor 5607 on the back lower arm.

In an example, an upper and/or lower arm can be (at least partially) transparent. In an example, an upper and/or lower arm can be compelled toward contact with a person's head by a spring and/or elastic mechanism. In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 57 shows an oblique left-side view of an example of a mobile wearable EEG device (e.g. electroencephalographic glasses) comprising: an eyewear frame with a longitudinal sidepiece (sometimes called a “temple”) 5701 which spans from a optical lens to a person's ear; a forehead loop (or band) 5704 which is connected to the longitudinal sidepiece, wherein the forehead loop extends forward and upward from a middle portion (e.g. the middle third of the length) of the longitudinal sidepiece and loops over (e.g. curves across) the person's forehead; a rear loop (or band) 5705 which is connected to the longitudinal sidepiece, wherein the rear loop extends backward and upward from a middle portion (e.g. the middle third of the length) of the longitudinal sidepiece and loops around the rear of the person's head; one or more electromagnetic energy sensors 5702 and 5703 on the forehead loop; and one or more electromagnetic energy sensors 5706 and 5707 on the rear loop.

In an example, this device can have lateral (right and left side) symmetry. In an example, a right side of the eyewear frame and loops (not shown in this diagram) can be symmetric to a left side of the eyewear frame and loops (shown in this diagram). In an example, a forehead loop can be connected to a right-side longitudinal sidepiece (not shown in this diagram) as well being connected to the left-side longitudinal sidepiece (shown in this diagram). In an example, a forehead loop can curve around the entire left-to-right width of a person's forehead. In an example, a rear loop can be connected to a right-side longitudinal sidepiece (not shown) as well as being connected to the left-side longitudinal sidepiece (which is shown). In an example, a rear loop can curve around the entire left-to-right width of the back of a person's head. In an example, a rear loop can loop around an upper-rear portion of a person's head at a height which is above the person's ear. In an example, a rear loop can loop around an upper-rear portion of a person's head at a height which is above the longitudinal sidepiece.

In an example, a forehead loop (or band) and a rear loop (or band) can be integrated into the frame of a smart eyewear product for mobile EEG monitoring. In another example, a forehead loop and a rear loop can comprise a separate, auxiliary device which clips to, clamps onto, plugs into, adheres to, or otherwise fastens onto the frame of conventional eyeglasses. The latter design enables a person to purchase a device with loops separately and to use this device with their existing (prescription) eyeglasses. In an example, there can be three electromagnetic energy sensors (e.g. a right-side sensor, a central sensor, and a left-side sensor) on a forehead loop. In an example, there can be three electromagnetic energy sensors (e.g. a right-side sensor, a central sensor, and a left-side sensor) on a rear loop.

In an example, a forehead loop can be (at least partially) transparent. In an example, a rear loop can be (at least partially) transparent. In an example, a forehead loop and/or a rear loop can be elastic or stretchable. In an example, a forehead loop and a rear loop can both be connected to a longitudinal sidepiece at the same location in the middle third of the length of the longitudinal sidepiece. In a variation on this example, a forehead loop and/or a rear loop can be connected to a longitudinal sidepiece on the rear third of the longitudinal sidepiece (e.g. at a location above the person's ear).

In an example, a forehead loop and/or a rear loop can be connected to a longitudinal sidepiece by a rotatable, pivoting, or otherwise-moveable joint. In an example, a forehead loop and/or a rear loop can be connected to a longitudinal sidepiece by a rotatable, pivoting, or otherwise-moveable axis. In an example, the angles at which the forehead loop and/or the rear loop are connected to the longitudinal sidepiece can be adjusted. In an example, a forehead loop can have a first configuration in which it loops around a person's forehead and a second configuration in which it loops around the top of the person's head, wherein the forehead loop can be moved from its first configuration to its second configuration, or vice versa. In an example, a forehead loop can have a first configuration in which it loops around a person's forehead and a second configuration in which it loops around the back of the person's head, wherein the forehead loop can be moved from its first configuration to its second configuration, or vice versa.

In an example, an electromagnetic energy sensor on a rear loop can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 58 shows an oblique left-side view of an example of a mobile wearable EEG device (e.g. electroencephalographic glasses) comprising: an eyewear frame with a longitudinal sidepiece (sometimes called a “temple”) 5801 which spans from a optical lens to a person's ear; a forehead loop (or band) 5804 which is connected to the longitudinal sidepiece, wherein the forehead loop (or band) extends forward and upward from a middle portion (e.g. the middle third of the length) of the longitudinal sidepiece and loops around (e.g. curves across) the person's forehead; wherein the forehead loop is connected to the longitudinal sidepiece by a rotatable, pivoting, or otherwise-moveable joint; wherein the forehead loop has a first configuration in which it loops around (e.g. curves across) a person's forehead and a second configuration in which it loops over the top of the person's head or around the back of the person's head, and wherein the forehead loop can be moved from its first configuration to its second configuration, or vice versa; and one or more electromagnetic energy sensors 5802 and 5803 on the forehead loop.

In an example, this device can have lateral (right and left side) symmetry. In an example, a right side of the eyewear frame and loops (not shown in this diagram) can be symmetric to a left side of the eyewear frame and loops (shown in this diagram). In an example, a forehead loop (or band) can be connected to a right-side longitudinal sidepiece (not shown in this diagram) as well being connected to the left-side longitudinal sidepiece (shown in this diagram). In an example, a forehead loop can curve around the entire left-to-right width of a person's forehead.

In an example, a forehead loop can be integrated into the frame of a smart eyewear product for mobile EEG monitoring. In an example, a forehead loop can be a separate, auxiliary device which clips to, clamps onto, plugs into, adheres to, or otherwise fastens onto the frame of conventional eyeglasses. The latter design enables a person to purchase a device (with loops) separately and to use this device with their existing (prescription) eyeglasses. In an example, there can be three electromagnetic energy sensors (e.g. a right-side sensor, a central sensor, and a left-side sensor) on a forehead loop.

In an example, a forehead loop can be (at least partially) transparent. In an example, a forehead loop can be elastic or stretchable. In a variation on this example, a forehead loop can be connected to a longitudinal sidepiece on the rear third of the longitudinal sidepiece (e.g. at a location above the person's ear). In an example, a forehead loop can be connected to a longitudinal sidepiece by a rotatable, pivoting, or otherwise-moveable joint. In an example, a forehead loop can be connected to a longitudinal sidepiece by a rotatable, pivoting, or otherwise-moveable axis. In an example, the angles at which the forehead loop are connected to the longitudinal sidepiece can be adjusted.

In an example, an electromagnetic energy sensor can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 59 shows an oblique left-side view of an example of a mobile wearable EEG device comprising: an arcuate headband 5903 which encircles a person's head and is removably attached to a longitudinal sidepiece 5901 (sometimes called a “temple”) of eyewear by at least one clip, clamp, snap, or plug 5904; at least one electromagnetic energy sensor 5902 on a front portion of the headband which spans the person's forehead; and at least one electromagnetic energy sensor 5905 on a rear portion of the headband which spans the back of the person's head. In an example, this device can have lateral (right and left side) symmetry.

The design of this device enables a person to purchase the device separately and to use it with their existing (prescription) eyeglasses. In an example, a headband can be connected to a right-side longitudinal sidepiece (not shown in this diagram) as well being connected to a left-side longitudinal sidepiece (shown in this diagram). In an example, a headband can be (at least partially) transparent. In an example, a headband can be elastic or stretchable. In an example, a headband can encircle a person's head in a substantially horizontal plane (e.g. within 20 degrees of horizontal when a person's head is upright). In an example, a (left or right) side of a headband can have a downward-facing convexity and/or an upward-facing concavity. In an example, a headband can have an elliptical or oval shape.

In an example, an electromagnetic energy sensor on a rear portion of the headband can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

FIG. 60 shows an oblique left-side view of another example of a mobile wearable EEG device comprising: an arcuate headband 6003 which encircles a person's head and is removably attached to a longitudinal sidepiece 6001 (sometimes called a “temple”) of eyewear by at least one clip, clamp, snap, or plug 6004; at least one electromagnetic energy sensor 6002 on a front portion of the headband which spans the person's forehead; and at least one electromagnetic energy sensor 6005 on a rear portion of the headband which spans the back of the person's head. In an example, this device can have lateral (right and left side) symmetry. In this example, the headband is higher than the longitudinal sidepiece except for a vertical connecting strip between the headband and the longitudinal sidepiece.

The design of this device enables a person to purchase the device separately and to use it with their existing (prescription) eyeglasses. In an example, a headband can be connected to a right-side longitudinal sidepiece (not shown in this diagram) as well being connected to a left-side longitudinal sidepiece (shown in this diagram). In an example, a headband can be (at least partially) transparent. In an example, a headband can be elastic or stretchable. In an example, a headband can encircle a person's head in a substantially horizontal plane (e.g. within 20 degrees of horizontal when a person's head is upright). In an example, a (left or right) side of a headband can have a downward-facing convexity and/or an upward-facing concavity. In an example, a headband can have an elliptical or oval shape.

In an example, an electromagnetic energy sensor on a rear portion of the headband can have protrusions (e.g. prongs, teeth, or combs) which protrude between strands of hair in order to be in contact the person's scalp and/or be in better electromagnetic communication with the person's brain. In an example, an electromagnetic energy sensor (e.g. electrode) can comprise a linear array of protrusions which extend between strands of hair to contact a person's scalp. In an example, an electromagnetic energy sensor (e.g. electrode) can comprise a two-dimensional array (e.g. matrix) of protrusions. In an example, an electrode can comprise two or more concentric rings of protrusions. In an example, protrusions can be tapered in a proximal to distal manner, wherein distal means closer to the person's head. In an example, a distal portion of a protrusion can be more conductive (e.g. have a higher concentration of conductive material) than a proximal portion of a protrusion. In an example, hair-penetrating protrusions on an electromagnetic energy sensor (e.g. electrode) can be made with a soft (e.g. elastomeric) conductive polymer-based material. In an example, an electromagnetic energy sensor can be made with a soft (e.g. elastomeric) conductive polymer-based material. In an example, soft conductive polymer-based material can be made from a polymer (e.g. polydimethylsiloxane, polybutylene terephthalate, or polyurethane) which has been impregnated, doped, filled, and/or coated with electro-conductive material (e.g. silver, carbon nanotubes or other forms of carbon, copper, aluminum, nickel, platinum, or gold). In an example, an electromagnetic energy sensor can contain (or be made with) hydrogel.

In an example, this device can further comprise a battery, a data processor, a wireless data transmitter, and a wireless data receiver. In an example, electromagnetic energy sensors can be EEG (electroencephalographic) sensors and/or electrodes. In an example, electromagnetic energy sensors can detect, measure, and/or record the electromagnetic activity of a person's brain. In an example, this electromagnetic activity can be analyzed in order to detect and/or predict (epileptic) seizures. In an example, this system can function as a BCI (Brain Computer Interface) for communication purposes, such as for people who have lost motor control (e.g. people with ALS) or for remote control of other devices. Example variations and components discussed elsewhere in this disclosure and in priority-linked disclosures can also be applied to this example where relevant.

The following example variations and components can be integrated, where relevant, into any of the examples shown in FIGS. 1 through 60. A mobile wearable device with electromagnetic energy sensors which is worn on a person's head can be used to collect data concerning electromagnetic brain activity. In an example, these electromagnetic energy sensors can be electroencephalographic (EEG) sensors. Such a mobile wearable device can include electromagnetic energy sensors which are in electromagnetic communication with the person's brain. In an example, electromagnetic energy sensors can measure the transmission of electromagnetic energy between two locations on a person's head and/or between layers of a material at a single location on the person's head. In an example, an electromagnetic energy sensor can measure electromagnetic energy conductivity, resistance, impedance, or capacitance. In an example, an electromagnetic energy sensor can comprise both conductive and non-conductive layers. In an example, an electromagnetic energy sensor can have a dielectric layer.

For mobile wearable devices, it is desirable that electromagnetic energy sensors be dry EEG sensors so that they do not require the application of conductive gel in order to be in electromagnetic communication with a person's brain. It is also desirable that these sensors be relatively soft, not having pressure points which are uncomfortable for extended wear. In an example, an electromagnetic energy sensor can be made from soft deformable conductive material which provides conformal contact with a person's head without creating irritating or painful points of high-pressure contact. In an example, an electromagnetic energy sensor can be made with conductive polydimethylsiloxane (PDMS), thermoplastic polyurethane (TPU), or poly(3,4-ethylenedioxythiophene):poly(styrene sulfonate) (PEDOT:PSS). In an example, the material can be hydrophilic.

In an example, an electromagnetic energy sensor can be made from elastic, stretchable, and/or deformable conductive material. In an example, an electromagnetic energy sensor can be made from elastic, stretchable, and/or deformable conductive material with a Shore A value which is less than 80. In an example, a first set of electromagnetic energy sensors (which are configured to be worn on a portion of a person's head which is not covered by hair) can have a lower average Shore value than a second set of electromagnetic energy sensors (which is configured to be worn on a portion of the person's head which is covered with hair). In an example, sensors which are configured to be worn on portions of a person's head which are covered with hair can comprise a plurality of hair-penetrating and/or hair-engaging protrusions, combs, teeth, or prongs which penetrate and/or engage layers of the person's hair.

In an example, a mobile wearable device can hold electromagnetic energy sensors in electromagnetic communication with a person's frontal lobe. In an example, a portion of a mobile wearable device can hold electromagnetic energy sensors on a person's forehead. In an example, a mobile wearable device can span a person's forehead. In an example, a mobile wearable device which spans a person's forehead can hold a first EEG sensor above a person's right eye and a second EEG sensor above a person's left eye. In an example, electromagnetic energy sensors which are configured to be worn on a person's forehead can have a low Shore A value (e.g. less than 80) so that they conform to the surface shape of the person's forehead for good electromagnetic communication with the person's frontal lobe. In an example, a portion of a device which spans a person's forehead can be transparent or translucent. In an example, a mobile wearable device can be a headband which encircles a person's head in a generally-horizontal manner, holding electromagnetic energy sensors in electromagnetic communication with both the person's frontal lobe and the person's occipital lobe.

In an example, a mobile wearable device can hold electromagnetic energy sensors in electromagnetic communication with a person's occipital lobe. In an example, a device can have a (semicircular) rear loop which curves around a portion of the rear half of a person's head. In an example, this loop can have a plurality of electromagnetic energy sensors which are in electromagnetic communication with the person's occipital lobe. In an example, data from these sensors can be analyzed to determine what a person is looking at, where the person is focusing, and/or an object of the person's attention. In an example, a mobile wearable device can hold one or more electromagnetic energy sensors in electromagnetic communication with a posterior portion of a person's head.

In an example, electromagnetic energy sensors which are configured to be worn on the posterior portion of a person's head can have a plurality of hair-penetrating and/or hair-engaging protrusions, combs, teeth, or prongs for good electromagnetic communication with the person's occipital lobe. These protrusions, combs, teeth, or prongs can protrude between strands of hair to contact the person's skin. In an example, electromagnetic energy sensors which are worn on the posterior a person's head can have a plurality of hair-engaging protrusions, combs, teeth, or prongs which hold the sensors in place by engaging the person's hair.

In an example, a wearable mobile device for monitoring electromagnetic brain activity can include an array of hair-penetrating conductive protrusions with a two-dimensional cross-sectional area in a plane which is substantially parallel to the surface of a person's head when the device is worn. In an example, protrusions toward the center of this cross-sectional area can be farther apart than protrusions toward the periphery of this cross-sectional area. In an example, protrusions toward the center of this cross-sectional area can be shorter than protrusions toward the periphery of this cross-sectional area. In an example, protrusions toward the center of this cross-sectional area can have a lower Shore A value than protrusions toward the periphery of this cross-sectional area. In an example, protrusions toward the center of this cross-sectional area can be less conductive than protrusions toward the periphery of this cross-sectional area.

In an example, a mobile wearable device can hold electromagnetic energy (EEG) sensors at one or more locations selected from the group of EEG placement sites consisting of: FP1, FPz, FP2, AF7, AF5, AF3, AFz, AF4, AF6, AF8, F7, F5, F3, F1, Fz, F2, F4, F6, F8, FT7, FC5, FC3, FC1, FCz, FC2, FC4, FC6, FT8, T3/T7, C3, C4, C1, Cz, C2, C5, C6, T4/T8, TP7, CP5, CP3, CP1, CPz, CP2, CP4, CP6, TP8, T5/P7, P5, P3, P1, Pz, P2, P4, P6, T6/P8, PO7, PO5, PO3, POz, PO4, PO6, PO8, O1, Oz, and O2.

In an example, a mobile wearable device can include a rubber cushion which gently holds an electromagnetic energy sensor against a person's head. In an example, a mobile wearable device can include a gel layer which gently holds an electromagnetic energy sensor against a person's head. In an example, a mobile wearable device can include compressive foam which gently holds an electromagnetic energy sensor against a person's head. In an example, a mobile wearable device can have a silicone-based layer which gently holds an electromagnetic energy sensor against a person's head. In an example, an electromagnetic energy sensor can be made with natural or synthetic sponge material. In an example, an electromagnetic energy sensor can be made with polydimethylsiloxane (PDMS), thermoplastic polyurethane (TPU), or poly(3,4-ethylenedioxythiophene):poly(styrene sulfonate) (PEDOT:PSS).

In an example, each electromagnetic energy sensor in a plurality of electromagnetic energy sensors can have its own rubber cushion, gel layer, or compressive foam which gently holds it against a person's head. In an example, the Shore A values of individual sensor cushions (or other compressive pieces) can be different for different individual electromagnetic energy sensors. In an example, electromagnetic energy sensors which are worn on a person's forehead can have softer cushions (or other compressive pieces) than electromagnetic energy sensors worn elsewhere on the person's head. In an example, a first set of electromagnetic energy sensors can be held against a person's head by a first set of cushions (or other compressive pieces) with a first average Shore A value, and a second set of electromagnetic energy sensors can be held against the person's head by a second set of cushions (or other compressive pieces) with a second average Shore A value, wherein the second average Shore A value is greater than the first average Shore A value.

In an example, a mobile wearable device can include one or more inflatable chambers (e.g. balloons or other type of inflatable chambers) which gently hold one or more electromagnetic energy sensors against a person's head. In an example, the inflation levels of these one or more chambers can be manually (and individually) adjusted by the person to adjust fit and/or comfort. In an example, the inflation levels of these one or more chambers can be automatically (and individually) adjusted by the device to optimize electromagnetic communication between the one or more sensors and the person's brain. In an example, a mobile wearable device can include one or more pneumatic chambers (e.g. pneumatic pistons) which gently hold one or more electromagnetic energy sensors against a person's head. In an example, the pressure levels of these one or more chambers can be manually (and individually) adjusted by the person to adjust fit and/or comfort. In an example, the pressure levels of these one or more chambers can be automatically (and individually) adjusted by the device to optimize electromagnetic communication between the one or more sensors and the person's brain.

In an example, a mobile wearable device can include one or more springs which hold one or more electromagnetic energy sensors gently against a person's head. In an example, the tensions and/or lengths of these one or more springs can be manually (and individually) adjusted by the person to adjust fit and/or comfort. In an example, the tensions and/or lengths of these one or more springs can be automatically adjusted by the device to optimize electromagnetic communication between the one or more sensors and the person's brain. In an example, a mobile wearable device can include one or more electromagnetic solenoids which hold one or more electromagnetic energy sensors gently against a person's head. In an example, the lengths or pressures of these one or more solenoids can be manually adjusted by the person to adjust fit and/or comfort. In an example, the lengths or pressures of these solenoids can be automatically adjusted by the device to optimize electromagnetic communication between the one or more sensors and the person's brain.

In an example, a mobile wearable device with one or more electromagnetic energy sensors can be embodied in an elastic band which is worn around a person's head. In an example, a mobile wearable device with one or more electromagnetic energy sensors can be embodied in an elastic textile band which is worn around a person's head. In an example, one or more electromagnetic energy sensors can be held in electromagnetic communication with a person's brain by a circular band (or strap) which is worn around a person's head. In an example, a mobile wearable device can be embodied in a headband. In an example, the headband can be generally circular, oval, or elliptical. In an example, the headband can encircle a person's head in a generally-horizontal plane when the person is standing upright. In an example, the headband can encircle a person's head in a plane which is tilted 5 to 30 degrees relative to a horizontal plane when the person is standing upright. In an example, the front portion of the headband can be ½″ to 3″ higher than the rear portion of the headband.

In an example, a mobile wearable device for measuring electromagnetic brain activity can be a relatively-elastic textile band. Alternatively, a mobile wearable device for measuring electromagnetic brain activity can be a relatively-rigid polymer band. In an example, a mobile wearable device which is worn around a person's head can have alternating elastic and rigid sections around its circumference. In an example, a mobile wearable device for measuring electromagnetic brain activity can comprise: a generally circular band which is worn around a person's head, wherein the circumference of the band further comprises a first set of partially-circumferential sections with a first level of elasticity, stretchability, or deformability and a second set of partially-circumferential sections with a second level of elasticity, stretchability, or deformability, wherein the second level is greater than the first level; and a plurality of electromagnetic energy sensors which are held on the person's head by the generally circular band.

In an example, a generally circular device which is worn on a person's head to measure electromagnetic brain activity can comprise: (a) a front (semicircular) loop which spans a person's forehead; wherein the front loop has a first level of elasticity, stretchability, or deformability; (b) a rear (semicircular) loop which spans the rear of the person's head; wherein the rear loop has a second level of elasticity, stretchability, or deformability; and wherein the second level is less than the first level; (c) a first set of electromagnetic energy sensors which are held in electromagnetic communication with the person's brain by the front loop; and (d) a second set of electromagnetic energy sensors which are held in electromagnetic communication with the person's brain by the rear loop.

In an example, a generally circular device which is worn on a person's head to measure electromagnetic brain activity can comprise: (a) a front (semicircular) loop which spans a person's forehead, wherein the front loop has a first Shore A value; (b) a rear (semicircular) loop which spans the rear of the person's head, wherein the rear loop has a second Shore A value; and wherein the second level is greater than the first level; (c) a first set of electromagnetic energy sensors which are held in electromagnetic communication with the person's brain by the front loop; and (d) a second set of electromagnetic energy sensors which are held in electromagnetic communication with the person's brain by the rear loop.

In an example, a mobile wearable device for measuring electromagnetic brain activity can comprise: a inner circular band which is worn around a person's head, wherein the inner circular band has a first level of elasticity, stretchability, or deformability; an outer circular band which is worn around the person's head, wherein the outer circular band has a second level of elasticity, stretchability, or deformability, wherein the second level is less than the first level; and a plurality of electromagnetic energy sensors which are held on the person's head by the inner circular band. In an example, a mobile wearable device for measuring electromagnetic brain activity can comprise: a inner semicircular band which is worn around a person's head, wherein the inner semicircular band has a first level of elasticity, stretchability, or deformability; an outer circular band which is worn around the person's head, wherein the outer circular band has a second level of elasticity, stretchability, or deformability, wherein the second level is less than the first level; and a plurality of electromagnetic energy sensors which are held on the person's head by the inner semicircular band.

In an example, a headband can be generally circular except for right-side and left-side undulating (e.g. sinusoidal or partially sinusoidal) sections which curve around upper portions of a person's right ear and left ear, respectively. In an example, a headband can be generally circular except for right-side and left-side undulating (e.g. sinusoidal or partially sinusoidal) sections which loop over upper portions of a person's right ear and left ear, respectively. In an example, a headband can be generally circular except for right-side and left-side undulating (e.g. sinusoidal or partially sinusoidal) sections which rest on top of the person's right and left ears, respectively. In an example, a headband can be generally circular except for right-side and left-side concave sections which curve around upper portions of the person's right and left ears, respectively. In an example, a headband can be generally circular except for right-side and left-side concave sections which loop over upper portions of the person's right and left ears, respectively. In an example, a headband can be generally circular except for right-side and left-side concave sections which rest on top of the person's right and left ears, respectively.

In an example, a mobile wearable device which is worn around a person's head can be a generally circular band which bifurcates and re-converges as it spans the person's ears. In an example, each side of the band can bifurcate into an upper branch which passes over the top of the ear and a lower branch which passes under the bottom of the ear. In an example, a mobile wearable device which is worn around a person's head can be generally circular, except that it locally bifurcates—with each side branching over and under the person's ears—and then re-converges. In an example, a mobile wearable device can comprise: right-side and left-side (circular, oval, or elliptical) ear loops, wherein each ear loop encircles an ear; a frontal loop which curves forward from the right-side and left-side ear loops and spans the person's forehead; a rear loop which curves backward from the right-side and left-side ear loops and span the rear of the person's head; and a plurality of electromagnetic energy (EEG) sensors.

In an example, a mobile wearable device with electromagnetic energy sensors for measuring electromagnetic brain activity can be made by weaving or knitting together a plurality of elastic and/or stretchable yarns (or threads). In an example, a mobile wearable device with electromagnetic energy sensors can be a headband which is made by weaving or knitting together soft, elastic, and/or stretchable yarns (or threads). In an example, some of these yarns (or threads) can be electroconductive. In an example, some of these yarns (or threads) can be made from previously non-conductive yarns (or threads) which have been coated or impregnated with conductive material. In an example, a mobile wearable device can be made weaving or knitting together: (a) non-conductive yarns, threads, or fibers; and (b) conductive yarns, threads, fibers, wires, or strands. In an example, a mobile wearable device can be made braiding and/or twisting together: (a) non-conductive yarns, threads, or fibers; and (b) conductive yarns, threads, fibers, wires, or strands. In an example, a headband can be made from soft, elastic, and/or stretchable fabric.

In an example, electroconductive yarns, threads, fibers, or strands can be made with concentric layers which have different conductivity levels. In an example, some concentric layers can be non-conductive and some concentric layers can be conductive. In an example, electroconductive yarns, threads, fibers, or strands can comprise a conductive core and a non-conductive outer layer. In an example, a yarn or thread can comprise: an inner core of twisted or braided conductive threads, fibers, or strands; and an outer layer of non-conductive material around the inner core. In an example, a conductive yarn or thread can comprise: an inner core of twisted or braided conductive and non-conductive threads, fibers, or strands; and an outer layer non-conductive material around the inner core.

In an example, yarns, threads, fibers, or strands can have concentric layers comprising: an inner concentric layer (e.g. a core) which is less conductive; a middle concentric layer which is around the core and is more conductive; and an outer concentric layer which is around the middle concentric layer which is less conductive. In an example, electroconductive yarns, threads, fibers, or strands can have concentric layers comprising: an inner concentric layer (e.g. a core) with a first conductivity level; a middle concentric layer with a second conductivity level which is around the core; and an outer concentric non-layer with a third conductivity level which is around the middle concentric layer, wherein the second conductivity level is greater than the first conductivity level, and wherein the second conductivity level is greater than the third conductivity level.

In an example, a headband can comprise: a first set of electromagnetic energy sensors which are worn on a person's forehead, wherein the first set of sensors are made from a soft, deformable, conductive polymer (such as conductive PDMS or TPU) and are configured to generally conform to the curvature of the person's forehead; a second set of electromagnetic energy sensors which are worn on the posterior of the person's head, wherein the second set of sensors further comprises a plurality of hair-penetrating protrusions. In an example, a headband can comprise: a first set of electromagnetic energy sensors which are worn on a person's forehead, wherein the first set of sensors are made from PEDOT:PSS and are configured to generally conform to the curvature of the person's forehead; a second set of electromagnetic energy sensors which are worn on the posterior of the person's head, wherein the second set of sensors further comprises a plurality of soft hair-penetrating protrusions.

In an example, a headband can comprise: a first set of electromagnetic energy sensors which are worn on a person's forehead, wherein sensors in this first set are made from a conductive polymer with a first durometer level and are configured to generally conform to the curvature of the person's forehead; a second set of electromagnetic energy sensors which are worn on the posterior of the person's head, wherein sensors in this second set further comprise a plurality of hair-penetrating protrusions made from a conductive polymer with a second durometer level, wherein the second durometer level is greater than the first durometer level. In an example, a headband can comprise: a first set of electromagnetic energy sensors which are worn on a person's forehead, wherein sensors in this first set are made from a conductive polymer with a first Shore value and are configured to generally conform to the curvature of the person's forehead; a second set of electromagnetic energy sensors which are worn on the posterior of the person's head, wherein sensors in this second set further comprise a plurality of hair-penetrating protrusions made from a conductive polymer with a second Shore value, wherein the second Shore value is greater than the first Shore value.

In an example, one or more electromagnetic energy sensors to measure brain activity can be embodied in a pair of EEG-monitoring headphones. In an example, these electromagnetic energy sensors can be in electromagnetic communication with a person's temporal lobe. In an example, these electromagnetic energy sensors can also be in electromagnetic communication with a person's parietal lobe. In an example, a pair of headphones can comprise: a first set of sensors which are worn within 4″ of a person's right side and left side ears, respectively, wherein this first set collects data concerning activity of the person's temporal lobe; and a second set of sensors which are worn over the top of the person's head and collect data concerning activity of the person's parietal lobe.

In an example, a pair of EEG-monitoring headphones can include electromagnetic energy sensors made from a deformable conductive polymer, such as conductive PDMS or TPU. In an example, a pair of EEG-monitoring headphones can include electromagnetic energy sensors made from PEDOT:PSS. In another example, a pair of EEG-monitoring headphones can include electromagnetic energy sensors which are knitted or woven from soft, elastic, and/or stretchable yarns and/or threads, wherein some of these yarns or threads are electroconductive.

In an example, a mobile wearable device for monitoring brain activity can be embodied in a “hearable” device, such as an ear bud, ear insert, or hearing aid. In an example, a device which is worn in, on, or around a person's ear can include one or more electromagnetic energy sensors which collect data concerning electromagnetic brain activity, especially activity of a person's temporal lobe. In an example, an EEG-monitoring ear-worn device can be custom-fitted to the contour of a person's ear canal for insertion into the person's ear canal. In an example, an EEG-monitoring ear-worn device can be sufficiently soft (e.g. low durometer) so that it deforms to the contour of a person's ear canal for insertion into the ear canal without custom fitting. In an example, an EEG-monitoring ear-worn device can include electromagnetic energy sensors made from a deformable conductive polymer, such as conductive PDMS or TPU. In an example, an EEG-monitoring ear-worn device can include electromagnetic energy sensors made from PEDOT:PSS.

In an example, an EEG-monitoring ear-worn device can be attached to a person's ear lobe. In an example, an EEG-monitoring ear-worn device can be embodied in an ear ring. In an example, an EEG-monitoring ear-worn device can loop around 50%-75% of the circumference of a person's outer ear. In an example, an EEG-monitoring ear-worn device can include: a first portion which loops around 50%-75% of the circumference of a person's outer ear; and a second portion which extends forward from the first portion to a location on the person's temple area and/or forehead. In an example, an EEG-monitoring ear-worn device can include: an ear-circling portion which loops around 50%-80% of the circumference of a person's outer ear; and an undulating arm which extends forward from the ear-circling portion to a location on the person's temple area and/or forehead. In an example, an EEG-monitoring ear-worn device can include: an ear-circling portion which loops around 50%-80% of the circumference of a person's outer ear; and a 2″ to 4″ arm which extends forward from the ear-circling portion to a location on a side of the person's forehead.

In an example, a wearable mobile device for measuring electromagnetic brain activity can be held on a person's head by frictional engagement with the person's hair. In an example, a wearable mobile device for measuring electromagnetic brain activity can be held on a person's head by clamping, clipping, and/or latching onto the person's hair. In an example, a wearable mobile device for measuring electromagnetic brain activity can be embodied in a hair comb, hair band, hair clip, or tiara. In an example, a wearable mobile device for measuring electromagnetic brain activity can be embodied in a hair comb, hair band, hair clip, or tiara which is worn over (and/or on) the top of a person's head. In an example, a wearable mobile device for measuring electromagnetic brain activity can be embodied in a hair comb, hair band, hair clip, or tiara which is worn around (and/or on) the rear a person's head.

In an example, frictional engagement with a person's hair can be provided by a plurality of hair-penetrating protrusions, prongs, or teeth which are inserted into a person's hair. In an example, frictional engagement with a person's hair can be provided by a plurality of hair-penetrating protrusions, prongs, or teeth which are inserted between layers of a person's hair. In an example, frictional engagement can be provided by one or more clips or clamps which are attached to a layer of person's hair. In an example, frictional engagement can be provided by one or more clips or clamps which grasp or otherwise engage strands of a person's hair.

In an example, a wearable mobile device for measuring brain activity can have a plurality of combs, prongs, teeth which extend upward under layers of a person's hair. In an example, a wearable mobile device for measuring brain activity can have a plurality of combs, prongs, teeth which extend downward into layers of a person's hair. In an example, portions of a wearable EEG device can rest on the tops of a person's outer ears and another portion can clamp, clip, and/or latch onto the person's hair. In an example, side segments of a wearable EEG device can rest on the tops of a person's right and left rears and a rear segment of the device can clamp, clip, and/or latch onto the person's hair.

In an example, frictional engagement between a device and a person's hair can be provided by one or more electromagnetic clamps which fasten to a layer of person's hair. In an example, frictional engagement between a device and a person's hair can be provided by one or more magnetic clamps which fasten to strands of a person's hair. In an example, a magnetic hair clamp for holding an EEG-monitoring device onto a person's head can be activated to engage hair by the application of electromagnetic energy to the clamp. In an example, the magnetic hair clamp can be disengaged from the hair by stopping the application of electromagnetic energy to the clamp.

In an example, a wearable device for monitoring electromagnetic brain activity can be embodied in a hat, baseball cap, skull cap, or helmet. In an example, a hat, baseball cap, skull cap, or helmet can have a plurality of electromagnetic energy sensors. In an example, a hat, baseball cap, skull cap, or helmet can have a plurality of soft textile-based electromagnetic energy sensors. In an example, a hat, baseball cap, skull cap, or helmet can have a plurality of soft conductive-polymer electromagnetic energy sensors. In an example, a hat, baseball cap, skull cap, or helmet can have a plurality of electromagnetic energy sensors made from conductive PDMS, TPU, or PEDOT:PSS. In an example, a hat, baseball cap, skull cap, or helmet can have electromagnetic energy sensors made by weaving or knitting together a plurality of soft, elastic, and/or stretchable conductive yarns or threads. In an example, a wearable device for monitoring electromagnetic brain activity can be removably attached to a hat, baseball cap, skill cap, or helmet. In an example, a wearable device for monitoring electromagnetic brain activity can be modular.

In an example, a wearable device for monitoring electromagnetic brain activity can be embodied in eyewear. In an example, a wearable device for monitoring electromagnetic brain activity can be embodied in a pair of eyeglasses. In an example, a wearable device for monitoring electromagnetic brain activity can be embodied in augmented reality (AR) or virtual reality (VR) eyewear. In an example, a wearable device for monitoring electromagnetic brain activity can be embodied in augmented reality (AR) or virtual reality (VR) eyeglasses. In an example, EEG eyewear can serve as a human-to-computer interface (e.g. BCI interface) for AR and/or VR applications.

In an example, one or more electromagnetic energy sensors can be located on the side-pieces (e.g. “temples”) and/or ear pieces of eyeglasses or other types of eyewear. In an example, one or more electromagnetic energy sensors can be located on arcuate (e.g. undulating or sinusoidal) side-pieces (e.g. “temples”) and/or ear pieces of eyeglasses or other eyewear. In an example, EEG eyewear (such as EEG eyeglasses) can have a plurality of electromagnetic energy sensors which are made from conductive PDMS, TPU, or PEDOT:PSS. In an example, EEG eyewear (such as EEG eyeglasses) can have a plurality of electromagnetic energy sensors which are made from hydrogel.

In an example, a wearable device for monitoring electromagnetic brain activity can be a modular eyewear accessory. In an example, a wearable device for monitoring electromagnetic brain activity can be removably-attached to a pair of eyeglasses. In an example, the device can be attached to eyewear via a clip or clamp. In an example, the device can be attached to eyewear via a strap, hook, or snap. In an example, the device can be attached to eyewear via a plug or pin. In an example, the device can be attached to eyewear via a magnet.

In an example, a wearable device for monitoring electromagnetic brain activity can be removably-attached to either the right sidepiece or the left sidepiece of a pair of eyeglasses. In an example, a wearable device for monitoring electromagnetic brain activity can be removably-attached to both the right and left sidepieces of a pair of eyeglasses. In an example, a modular wearable device with one or more electromagnetic energy sensors can be removably clipped or clamped onto the sidepiece of a pair of eyeglasses, such that the sensors are brought into electromagnetic energy communication with the side of a person's forehead and/or the person's temple area. In an example, a modular wearable device with one or more electromagnetic energy sensors can be removably slipped over the sidepiece of a pair of eyeglasses.

In an example, a wearable device for measuring brain activity can be embodied in an elastic band whose ends are connected to the right-side and left-side ear loops of a pair of eyeglasses and which curves around the rear of a person's head. In an example, a wearable device for measuring brain activity can be embodied in an elastic band whose ends are connected to the right-side and left-side sidepieces of a pair of eyeglasses and which curves over the top of a person's head. In an example, a wearable device for measuring brain activity can be embodied in an elastic band whose ends are connected to the right-side and left-side sidepieces of a pair of eyeglasses and which curves around a person's forehead.

In an example, a wearable device for monitoring electromagnetic brain activity can be a (semi-circular) rear loop which attaches to both the right and left sidepieces of a pair of eyeglasses and curves around the rear of the person's head. In an example, a wearable device for monitoring electromagnetic brain activity can be an elastic (semi-circular) band loop which attaches to both the right and left sidepieces of a pair of eyeglasses and curves around the rear of the person's head. In an example, this loop can curve around the rear of a person's head and hold one or more electromagnetic energy sensors in electromagnetic communication with the person's occipital lobe. In an example, these electromagnetic energy sensors can further comprise hair-penetrating and/or hair-engaging conductive protrusions, prongs, combs, or teeth. In an example, an eyewear accessory device which is removably attached to eyewear can comprise electromagnetic energy sensors which are made from conductive PDMS, TPU, or PEDOT:PSS. In an example, an eyewear accessory device which is removably attached to eyewear can comprise electromagnetic energy sensors which are made from a hydrogel.

In an example, a mobile wearable device with electromagnetic energy sensors for monitoring brain activity can be embodied in an eyewear accessory which is removably attached to eyewear (such as a pair of eyeglasses or AR/VR eyewear). In an example, such an eyewear accessory can be removably attached to a sidepiece (e.g. “temple”) of eyewear with a clip, clasp, magnet, strap, hook, or plug. In an example, such an eyewear accessory can be removably attached to the ear-engaging end of a sidepiece (e.g. “temple”) of eyewear. In an alternative example, such an eyewear accessory can slide over the ear-engaging end of a sidepiece (e.g. “temple”) of eyewear.

In an example, such an eyewear accessory can include one or more compressive members (e.g. pieces of compressible foam or gel) between the sidepiece and the surface of the person's head, wherein these one or more compressive members gently hold one or more electromagnetic energy sensors against the surface of the person's head. In an example, such an eyewear accessory can include one or more adjustably-inflatable compartments between the sidepiece and the surface of the person's head, wherein these one or more adjustably-inflatable compartments gently hold one or more electromagnetic energy sensors against the surface of the person's head. In an example, such an eyewear accessory can include one or more springs between the sidepiece and the surface of the person's head, wherein these one or more springs gently hold one or more electromagnetic energy sensors against the surface of the person's head.

In an example, a mobile wearable device with electromagnetic energy sensors for monitoring brain activity can be embodied in an eyewear accessory which spans across a person's forehead from the right sidepiece of a pair of eyeglasses to the left sidepiece of the pair of eyeglasses. In an example, such a forehead-spanning eyewear accessory can hold one of more electromagnetic energy sensors in electromagnetic communication with a person's frontal lobe. In an example, such a forehead-spanning eyewear accessory can be removably attached to the sidepieces of a pair of eyeglasses with clips, clamps, hooks, straps, loops, magnets, or plugs. In an example, some or all of such a forehead-spanning eyewear accessory can be transparent.

In an example, a wearable device for monitoring electromagnetic brain activity can be embodied in a respiratory mask. In an example, a wearable device for monitoring electromagnetic brain activity can be embodied in a CPAP mask. In an example, one or more electromagnetic energy sensors which collect data concerning brain activity can be part of a respiratory mask. In an example, one or more electromagnetic energy sensors can be held in electromagnetic communication with a person's brain by a respiratory (e.g. CPAP) mask. In an example, a respiratory (e.g. CPAP) mask can include electromagnetic energy sensors which are made from conductive PDMS, TPU, or PEDOT:PSS. In an example, a respiratory (e.g. CPAP) mask can include electromagnetic energy sensors which are made from a hydrogel.

In an example, an electromagnetic energy sensor can comprise a capacitor which is made by bonding together alternating layers of conductive and non-conductive polymers. In an example, an electromagnetic energy sensor can be made by 3D printing in which conductive ink is printed onto fabric. In another example, an electromagnetic energy sensor can be made by adhering (or otherwise bonding) together a first flexible non-conductive layer, a flexible conductive layer, and a second flexible non-conductive layer; wherein the flexible conductive layer is between the first flexible non-conductive layer and the second flexible non-conductive layer.

In an example, an electromagnetic energy sensor (such as an EEG sensor) can be formed by adhering (or otherwise bonding) an elastomeric conductive polymer to the inner (e.g. body-facing) surface of a semicircular band; wherein the semicircular band loops over the top of a person's head. In one embodiment, an electromagnetic energy sensor can be made by adhering and/or bonding flexible conductive patches onto the inner (e.g. body-facing) surface of a headband. Alternatively, an electromagnetic energy sensor can be made by adhering and/or bonding conductive hydrogel electrodes onto the inner (e.g. body-facing) surface of a headband. In one embodiment, an electromagnetic energy sensor can be made by printing conductive ink onto the inner (e.g. body-facing) surface of a headband. In one embodiment, an electromagnetic energy sensor can be made by printing elastomeric conductive ink onto the inner (e.g. body-facing) surface of a flexible textile headband.

In another example, an electromagnetic energy sensor can be made by coating a plurality of flexible hair-penetrating protrusions with conductive ink or paint. In an example, an electromagnetic energy sensor can be made by dipping a hair comb into conductive material. In an example, an electromagnetic energy sensor can be formed by embroidering or stitching a sinusoidal or zigzag pattern with conductive threads or yarns onto an article of clothing. In an example, an electromagnetic energy sensor (such as an EEG sensor) can be made by embroidering or stitching onto a headband using conductive threads or yarns. In another example, an electromagnetic energy sensor can be made by injection molding with a conductive elastomeric polymer. In one embodiment, an electromagnetic energy sensor (such as an EEG sensor) can be made by printing (or otherwise adhering) flexible conductive hair-penetrating protrusions onto the inner (e.g. body-facing) surface of a headband.

In another embodiment, an electromagnetic energy sensor can be made by printing conductive carbon-based ink onto fabric in a sinusoidal or zigzag pattern. In an example, an electromagnetic energy sensor can be made by printing conductive elastomeric ink onto the inner (e.g. body-facing) side of a headband. In another example, an electromagnetic energy sensor can be formed by printing conductive graphene-containing ink onto fabric. Alternatively, an electromagnetic energy sensor can be made by printing conductive ink onto the inner (e.g. body-facing) side of a headband. In an example, an electromagnetic energy sensor can be made by printing conductive silver-based ink onto fabric in a sinusoidal or zigzag pattern. In one embodiment, an electromagnetic energy sensor (such as an EEG sensor) can be made by printing conductive traces onto fabric with conductive ink.

In another example, an electromagnetic energy sensor can be made by printing, coating, and/or spraying a layer of elastic conductive material onto a layer of elastic non-conductive material. In an example, an electromagnetic energy sensor (such as an EEG sensor) can be formed by printing, coating, and/or spraying conductive material onto a non-conductive hair-penetrating protrusion. In an example, an electromagnetic energy sensor can be made by printing, coating, and/or spraying conductive material onto a plurality of non-conductive flexible hair-penetrating protrusions. In another example, an electromagnetic energy sensor can be made by printing, coating, painting, or spraying carbon-based ink or particles onto low-conductivity material. In an example, an electromagnetic energy sensor can be made by printing, coating, painting, or spraying silver chloride onto low-conductivity material.

In another embodiment, an electromagnetic energy sensor can be made by printing, spraying, or adhering a layer of conductive material onto a non-conductive core of a flexible hair-penetrating protrusion. In one embodiment, an electromagnetic energy sensor can be formed by printing, spraying, or adhering a layer of conductive TPU (e.g. TPU which has been doped with conductive material) onto the inner (e.g. body-facing) surface of a headband. Alternatively, an electromagnetic energy sensor (such as an EEG sensor) can be made by screen printing with conductive ink onto an article of clothing. In an example, an electromagnetic energy sensor can be formed by spraying a plurality of flexible hair-penetrating protrusions with conductive material. In another example, an electromagnetic energy sensor (such as an EEG sensor) can be made by stitching conductive threads or yarns onto fabric in a sinusoidal and/or zigzag pattern. In an example, an electromagnetic energy sensor can be formed by weaving or knitting electrodes in a sinusoidal pattern using conductive threads or yarns. In one embodiment, an electromagnetic energy sensor can be made by weaving an orthogonal mesh with conductive and non-conductive threads or yarns.

In an example, an electromagnetic energy sensor can be a capacitor which is made by bonding together alternating layers of conductive and non-conductive elastomeric material. In another example, an electromagnetic energy sensor can be made by 3D printing, wherein conductive ink is printed onto a layer of non-conductive material. Alternatively, an electromagnetic energy sensor can be made by adhering (or otherwise bonding) an elastomeric conductive polymer to the inner (e.g. body-facing) surface of a circular headband. In an example, an electromagnetic energy sensor can be formed by adhering (or otherwise bonding) an elastomeric conductive polymer to the inner (e.g. body-facing) surface of a semicircular band; wherein the band curves around the posterior half of a person's head from one ear to the other ear. In one embodiment, an electromagnetic energy sensor (such as an EEG sensor) can be made by adhering and/or bonding conductive polymer patches onto the inner (e.g. body-facing) surface of a headband. In another example, an electromagnetic energy sensor can be formed by adhering and/or bonding conductive PEDOT:PSS electrodes onto the inner (e.g. body-facing) surface of a headband.

In an example, an electromagnetic energy sensor (such as an EEG sensor) can be made by coating a hair comb or hair band with conductive ink or paint. In another embodiment, an electromagnetic energy sensor can be formed by dipping a hair comb into conductive ink or paint. In an example, an electromagnetic energy sensor can be made by embroidering or stitching an orthogonal mesh of conductive threads or yarns. In an example, an electromagnetic energy sensor can be made by embroidering or stitching onto a hat or cap using conductive threads or yarns. In one embodiment, an electromagnetic energy sensor can be made by injection molding with a silicone-based polymer.

In another example, an electromagnetic energy sensor can be made by printing (or otherwise adhering) alternating layers of conductive and non-conductive polymers. Alternatively, an electromagnetic energy sensor (such as an EEG sensor) can be made by printing conductive carbon-based ink onto an article of clothing in a sinusoidal or zigzag pattern. In another example, an electromagnetic energy sensor can be made by printing conductive graphene-containing ink onto a layer of non-conductive material. In an example, an electromagnetic energy sensor (such as an EEG sensor) can be formed by printing conductive graphene-containing ink. Alternatively, an electromagnetic energy sensor can be made by printing electro-conductive pathways onto fabric with conductive ink. In one embodiment, an electromagnetic energy sensor can be formed by printing conductive silver-based ink onto an article of clothing in a sinusoidal or zigzag pattern. In an example, an electromagnetic energy sensor can be made by printing electrodes onto a layer of non-conductive material with conductive ink.

In another example, an electromagnetic energy sensor can be made by printing, coating, and/or spraying a layer of flexible conductive material onto a layer of flexible non-conductive material. In one embodiment, an electromagnetic energy sensor can be made by printing, coating, and/or spraying conductive material onto a non-conductive polymer hair-penetrating protrusion. In another example, an electromagnetic energy sensor can be made by printing, coating, and/or spraying conductive material onto a plurality of non-conductive hair-penetrating prongs, teeth, or combs. In an example, an electromagnetic energy sensor can be formed by printing, coating, painting, or spraying conductive ink onto low-conductivity material. In an example, an electromagnetic energy sensor (such as an EEG sensor) can be made by printing, coating, painting, or spraying silver-based ink or particles onto low-conductivity material.

Alternatively, an electromagnetic energy sensor can be made by printing, spraying, or adhering a layer of conductive material onto a layer of non-conductive material. In another embodiment, an electromagnetic energy sensor (such as an EEG sensor) can be made by printing, spraying, or adhering a layer of conductive hydrogel onto the inner (e.g. body-facing) surface of a headband. In an example, an electromagnetic energy sensor can be made by screen printing onto a layer of non-conductive material with conductive ink. In another example, an electromagnetic energy sensor can be formed by spraying a plurality of flexible hair-penetrating protrusions with conductive ink or paint. In one embodiment, an electromagnetic energy sensor can be made by stitching conductive threads or yarns onto fabric in an orthogonal mesh. Alternatively, an electromagnetic energy sensor can be made by weaving or knitting electrodes in an orthogonal pattern using conductive threads or yarns.

In one embodiment, an electromagnetic energy sensor can be a dielectric structure which is made by bonding together alternating layers of conductive and non-conductive polymers. In another embodiment, an electromagnetic energy sensor (such as an EEG sensor) can be made by 3D printing, wherein conductive ink is printed onto an article of clothing. In an example, an electromagnetic energy sensor can be formed by adhering (or otherwise bonding) an elastomeric conductive polymer to the inner (e.g. body-facing) surface of a semicircular band; wherein the band curves around the posterior half of a person's head. In another example, an electromagnetic energy sensor (such as an EEG sensor) can be made by adhering (or otherwise bonding) an elastomeric conductive polymer to the inner (e.g. body-facing) surface of a semicircular band; wherein the band curves around the anterior half of a person's head from one ear to the other ear. In an example, an electromagnetic energy sensor can be formed by adhering and/or bonding conductive PDMS electrodes onto the inner (e.g. body-facing) surface of a headband.

In an example, an electromagnetic energy sensor can be made by bonding together alternating layers of conductive and non-conductive polymers. Alternatively, an electromagnetic energy sensor can be made by dipping a plurality of flexible hair-penetrating protrusions into conductive material. In another example, an electromagnetic energy sensor can be made by embroidering or stitching a sinusoidal or zigzag pattern with conductive threads or yarns. In one embodiment, an electromagnetic energy sensor can be made by embroidering or stitching an orthogonal mesh with conductive threads or yarns onto fabric. In another example, an electromagnetic energy sensor (such as an EEG sensor) can be formed by extruding and curing alternating layers of conductive and non-conductive material. In an example, an electromagnetic energy sensor can be made by pouring and curing alternating layers of conductive and non-conductive elastic polymers.

In one embodiment, an electromagnetic energy sensor (such as an EEG sensor) can be formed by printing conductive carbon-based ink onto a layer of non-conductive material. In an example, an electromagnetic energy sensor can be made by printing conductive carbon-based ink onto fabric. In another embodiment, an electromagnetic energy sensor can be formed by printing conductive graphene-containing ink onto a layer of non-conductive material in a sinusoidal or zigzag pattern. In an example, an electromagnetic energy sensor can be made by printing conductive ink onto fabric. In another example, an electromagnetic energy sensor can be made by printing conductive silver-based ink onto a layer of non-conductive material. Alternatively, an electromagnetic energy sensor can be made by printing conductive silver-based ink onto fabric.

In an example, an electromagnetic energy sensor can be made by printing electrodes onto fabric with conductive ink. In one embodiment, an electromagnetic energy sensor can be made by printing, coating, and/or spraying carbon-based conductive ink onto a non-conductive hair-penetrating protrusion. In another example, an electromagnetic energy sensor (such as an EEG sensor) can be made by printing, coating, and/or spraying conductive material onto the non-conductive core of a flexible hair-penetrating protrusion. In an example, an electromagnetic energy sensor can be made by printing, coating, and/or spraying non-conductive material onto conductive flexible hair-penetrating protrusions. In another example, an electromagnetic energy sensor (such as an EEG sensor) can be formed by printing, coating, painting, or spraying copper-based ink or particles onto low-conductivity material.

Alternatively, an electromagnetic energy sensor can be made by printing, coating, painting, or spraying steel particles onto low-conductivity material. In an example, an electromagnetic energy sensor can be made by printing, spraying, or adhering a layer of conductive material onto a non-conductive core of a hair-penetrating protrusion. In one embodiment, an electromagnetic energy sensor can be made by printing, spraying, or adhering a layer of elastomeric conductive polymer material onto the inner (e.g. body-facing) surface of a headband.

Alternatively, an electromagnetic energy sensor can be made by sewing conductive threads or yarns onto fabric in a sinusoidal and/or zigzag pattern. In another embodiment, an electromagnetic energy sensor can be made by spraying a hair comb or hair band with conductive material. In one embodiment, an electromagnetic energy sensor can be formed by stitching electrodes with conductive threads or yarns. In another example, an electromagnetic energy sensor (such as an EEG sensor) can be made by weaving a mesh of conductive threads or yarns with a first orientation and non-conductive threads or yarns in a second direction; wherein the second direction is orthogonal to the first direction.

In an example, an electromagnetic energy sensor can be a dielectric structure which is formed by bonding together alternating layers of conductive and non-conductive elastomeric material. In another example, an electromagnetic energy sensor (such as an EEG sensor) can be made by adhering (or otherwise bonding) a first flexible conductive layer, a flexible non-conductive layer, and a second flexible conductive layer; wherein the flexible non-conductive layer is between the first flexible conductive layer and the second flexible conductive layer. In an example, an electromagnetic energy sensor can be made by adhering (or otherwise bonding) an elastomeric conductive polymer to the inner (e.g. body-facing) surface of a semicircular band; wherein the band curves around the anterior half of a person's head. In an example, an electromagnetic energy sensor can be made by adhering (or otherwise bonding) an elastomeric conductive polymer to the inner (e.g. body-facing) surface of a semicircular band; wherein the band curves over the top of a person's head from one ear to the other ear. In one embodiment, an electromagnetic energy sensor can be made by adhering and/or bonding conductive TPU electrodes onto the inner (e.g. body-facing) surface of a headband.

In another example, an electromagnetic energy sensor can be made by bonding together alternating layers of conductive and non-conductive elastomeric material. In an example, an electromagnetic energy sensor can be formed by dipping a plurality of flexible hair-penetrating protrusions into conductive ink or paint. In another embodiment, an electromagnetic energy sensor can be made by embroidering or stitching a sinusoidal or zigzag pattern with conductive threads or yarns onto fabric. In one embodiment, an electromagnetic energy sensor can be made by embroidering or stitching an orthogonal mesh with conductive threads or yarns onto an article of clothing. In an example, an electromagnetic energy sensor can be made by filling a non-conductive lumen with a conductive liquid.

In an example, an electromagnetic energy sensor (such as an EEG sensor) can be made by pouring and curing alternating layers of conductive and non-conductive low modulus polymers. In another example, an electromagnetic energy sensor can be formed by printing conductive carbon-based ink onto a layer of non-conductive material in a sinusoidal or zigzag pattern. Alternatively, an electromagnetic energy sensor can be made by printing with conductive carbon-based ink. In another example, an electromagnetic energy sensor can be formed by printing conductive graphene-containing ink onto fabric in a sinusoidal or zigzag pattern. In one embodiment, an electromagnetic energy sensor can be made by printing conductive ink onto non-conductive material. In another example, an electromagnetic energy sensor can be made by printing conductive silver-based ink onto a layer of non-conductive material in a sinusoidal or zigzag pattern. In an example, an electromagnetic energy sensor can be made by printing conductive silver-based ink.

Alternatively, an electromagnetic energy sensor can be made by printing, coating, and/or spraying a layer of conductive material onto a non-conductive longitudinal hair-penetrating protrusion. In an example, an electromagnetic energy sensor (such as an EEG sensor) can be made by printing, coating, and/or spraying conductive material onto a longitudinal non-conductive core of a hair-penetrating protrusion. In another embodiment, an electromagnetic energy sensor can be made by printing, coating, and/or spraying conductive material onto a non-conductive hair-penetrating protrusion. In one embodiment, an electromagnetic energy sensor can be formed by printing, coating, and/or spraying silver-based conductive ink onto a non-conductive hair-penetrating protrusion. In another example, an electromagnetic energy sensor can be made by printing, coating, painting, or spraying gold particles onto low-conductivity material.

In an example, an electromagnetic energy sensor can be formed by printing, spraying, or adhering a layer of conductive hydrogel material onto the inner (e.g. body-facing) surface of a headband. In an example, an electromagnetic energy sensor (such as an EEG sensor) can be made by printing, spraying, or adhering a layer of conductive PDMS (e.g. PDMS which has been doped with conductive material) onto the inner (e.g. body-facing) surface of a headband. Alternatively, an electromagnetic energy sensor can be made by screen printing with conductive ink onto fabric. In an example, an electromagnetic energy sensor can be made by sewing conductive threads or yarns onto fabric in an orthogonal mesh. In another example, an electromagnetic energy sensor can be made by spraying a hair comb or hair band with conductive ink or paint. In an example, an electromagnetic energy sensor can be formed by weaving or knitting electrodes using conductive threads or yarns. In another example, an electromagnetic energy sensor can be made by weaving electrodes with conductive threads or yarns.

In an example, a deformable, flexible, conductive electromagnetic energy sensor can comprise one or more protrusions, prongs, teeth, combs, pins, and/or petals which slide between strands of a person's hair so that the sensor is in electromagnetic communication with a person's brain despite being on a portion of the person's head which is covered by hair. In an example, a deformable, flexible, conductive electromagnetic energy sensor can comprise one or more protrusions, prongs, teeth, combs, pins, and/or petals which engage with a person's hair so that the sensor is in electromagnetic communication with a person's brain despite being on a portion of the person's head which is covered by hair. In another example, a deformable, flexible, conductive electromagnetic energy sensor can comprise one or more protrusions, prongs, teeth, combs, pins, and/or petals which intermesh and/or interdigitate with a person's hair so that the sensor is in electromagnetic communication with a person's brain despite being on a portion of the person's head which is covered by hair.

In another example, a deformable, flexible, conductive electromagnetic energy sensor can be configured to be in electromagnetic communication with a person's brain despite the person's hair. In an embodiment, a electromagnetic energy sensor can comprise one or more deformable, flexible, and conductive prongs which protrude between strands of a person's hair so that the sensor is in electromagnetic communication with a person's brain despite being on a portion of the person's head which is covered by hair. In another example, an electromagnetic energy sensor can be peanut shaped. In another example, an electromagnetic energy sensor can comprise a hair-penetrating protrusion which is tapered with the smaller section being closer to a person's head.

In an example, an electromagnetic energy sensor can have a dumbbell shape. Alternatively, an electromagnetic energy sensor can have a linear shape. In an embodiment, an electromagnetic energy sensor can have a parabolic shape whose convex surface protrudes between strands of a person's hair. In an example, an electromagnetic energy sensor can have an arcuate or round shape. In another example, an electromagnetic energy sensor can have a sawtooth shape whose peaks protrude into a person's hair. In another example, an electromagnetic energy sensor can have a helical shape. In an example, an electromagnetic energy sensor can comprise a plurality of helical hair-penetrating conductive protrusions.

In an example, an electromagnetic energy sensor can include a hair-penetrating protrusion with a circular cross-sectional shape in a plane which is orthogonal to the surface of a person's head. In another example, an electromagnetic energy sensor can include a hair-penetrating protrusion with a linear cross-sectional shape in a plane which is parallel to the surface of a person's head. In another example, an electromagnetic energy sensor can comprise a hair-penetrating protrusion with a square cross-sectional shape in a plane which is orthogonal to the surface of a person's head. In an embodiment, an electromagnetic energy sensor can include a hair-penetrating protrusion with a tapered cross-sectional shape in a plane which is parallel to the surface of a person's head. Alternatively, an electromagnetic energy sensor can include a hair-penetrating protrusion with an arcuate cross-sectional shape in a plane which is parallel to the surface of a person's head.

In an embodiment, a deformable, flexible, conductive electromagnetic energy sensor can comprise one or more protrusions, prongs, teeth, combs, pins, and/or petals which slide into and/or under a layer of person's hair so that the sensor is in electromagnetic communication with a person's brain despite being on a portion of the person's head which is covered by hair. In another example, a deformable, flexible, conductive electromagnetic energy sensor can comprise one or more protrusions, prongs, teeth, combs, pins, and/or petals which interlock with a person's hair so that the sensor is in electromagnetic communication with a person's brain despite being on a portion of the person's head which is covered by hair. In an example, a deformable, flexible, conductive electromagnetic energy sensor can comprise one or more protrusions, prongs, teeth, combs, pins, and/or petals which frictionally engage a person's hair so that the sensor is in electromagnetic communication with a person's brain despite being on a portion of the person's head which is covered by hair.

In an example, an electromagnetic energy sensor can comprise a plurality of hair-penetrating conductive protrusions which extend toward the surface of a person's head in a perpendicular (or orthogonal) manner. In an example, an electromagnetic energy sensor can comprise a plurality of hair-penetrating conductive protrusions which extend toward the surface of a person's head at an acute angle. In an example, an electromagnetic energy sensor can comprise a plurality of hair-penetrating conductive protrusions which are inserted between layers of a person's hair at an acute angle relative to the surface of the person's head.

In another example, a deformable, flexible, conductive electromagnetic energy sensor can be configured to be in electromagnetic communication with a person's brain despite being located on a portion of the person's head which is covered by hair. In an embodiment, a electromagnetic energy sensor can comprise one or more deformable, flexible, and conductive protrusions which protrude between strands of a person's hair so that the sensor is in electromagnetic communication with a person's brain despite being on a portion of the person's head which is covered by hair. In an example, an electromagnetic energy sensor can comprise a parabolic hair-penetrating protrusion which is tapered with the smaller section being closer to a person's head.

In an example, an electromagnetic energy sensor can have a polygonal shape. In another example, an electromagnetic energy sensor can have a rounded-rectangular shape. Alternatively, an electromagnetic energy sensor can have a sinusoidal shape whose peaks protrude into a person's hair. In another example, an electromagnetic energy sensor can have an hour-glass shape. In an example, an electromagnetic energy sensor can be circular.

In an embodiment, an electromagnetic energy sensor can include a hair-penetrating protrusion with a circular cross-sectional shape in a plane which is parallel to the surface of a person's head. Alternatively, an electromagnetic energy sensor can include a hair-penetrating protrusion with a hexagonal cross-sectional shape in a plane which is orthogonal to the surface of a person's head. In another example, an electromagnetic energy sensor can comprise a hair-penetrating protrusion with a polygonal cross-sectional shape in a plane which is orthogonal to the surface of a person's head. In an embodiment, an electromagnetic energy sensor can include a hair-penetrating protrusion with a square cross-sectional shape in a plane which is parallel to the surface of a person's head. In another example, an electromagnetic energy sensor can include a hair-penetrating protrusion with an annular cross-sectional shape in a plane which is orthogonal to the surface of a person's head. In an example, an electromagnetic energy sensor can comprise a hair-penetrating protrusion with an oval or elliptical cross-sectional shape in a plane which is orthogonal to the surface of a person's head.

In an embodiment, a deformable, flexible, conductive electromagnetic energy sensor can comprise one or more protrusions, prongs, teeth, combs, pins, and/or petals which protrude, penetrate, and/or extend between strands of a person's hair so that the sensor is in electromagnetic communication with a person's brain despite being on a portion of the person's head which is covered by hair. Alternatively, a deformable, flexible, conductive electromagnetic energy sensor can comprise one or more protrusions, prongs, teeth, combs, pins, and/or petals which slide upward under a layer of a person's hair so that the sensor is in electromagnetic communication with a person's brain despite being on a portion of the person's head which is covered by hair. In another example, a deformable, flexible, conductive electromagnetic energy sensor can comprise one or more protrusions, prongs, teeth, combs, pins, and/or petals which clamp onto a person's hair so that the sensor is in electromagnetic communication with a person's brain despite being on a portion of the person's head which is covered by hair.

In an example, a electromagnetic energy sensor can comprise one or more deformable, flexible, and conductive micro-columns which protrude between strands of a person's hair so that the sensor is in electromagnetic communication with a person's brain despite being on a portion of the person's head which is covered by hair. In another example, an electromagnetic energy sensor can comprise a sinusoidal hair-penetrating protrusion which is tapered with the smaller section being closer to a person's head.

In an example, an electromagnetic energy sensor can comprise concentric conductive rings. In an example, an electromagnetic energy sensor can comprise nested conductive rings. In an embodiment, an electromagnetic energy sensor can have a polyhedral shape. In another example, an electromagnetic energy sensor can have a square shape. Alternatively, an electromagnetic energy sensor can have a zigzag shape whose peaks protrude into a person's hair. In another example, an electromagnetic energy sensor can have an oval or elliptical shape.

In an embodiment, an electromagnetic energy sensor can include a hair-penetrating protrusion with a conic-section-shaped cross-sectional shape in a plane which is parallel to the surface of a person's head. In an example, an electromagnetic energy sensor can include a hair-penetrating protrusion with a hexagonal cross-sectional shape in a plane which is parallel to the surface of a person's head. In an example, an electromagnetic energy sensor can include a hair-penetrating protrusion with a polygonal cross-sectional shape in a plane which is parallel to the surface of a person's head. In an example, an electromagnetic energy sensor can comprise a hair-penetrating protrusion with a square cross-sectional shape in a plane which is parallel to the surface of a person's head.

Alternatively, an electromagnetic energy sensor can include a hair-penetrating protrusion with an annular cross-sectional shape in a plane which is parallel to the surface of a person's head. In another example, an electromagnetic energy sensor can comprise a hair-penetrating protrusion with an oval or elliptical cross-sectional shape in a plane which is parallel to the surface of a person's head. In an example, a deformable, flexible, conductive electromagnetic energy sensor can comprise one or more protrusions, prongs, teeth, combs, pins, and/or petals which are inserted between strands of a person's hair so that the sensor is in electromagnetic communication with a person's brain despite being on a portion of the person's head which is covered by hair.

In another example, a deformable, flexible, conductive electromagnetic energy sensor can comprise one or more protrusions, prongs, teeth, combs, pins, and/or petals which latch onto a person's hair so that the sensor is in electromagnetic communication with a person's brain despite being on a portion of the person's head which is covered by hair. Alternatively, a deformable, flexible, conductive electromagnetic energy sensor can comprise one or more protrusions, prongs, teeth, combs, pins, and/or petals which latch onto or engage with strands of a person's hair so that the sensor is in electromagnetic communication with a person's brain despite being on a portion of the person's head which is covered by hair.

In an example, a electromagnetic energy sensor can comprise one or more deformable, flexible, and conductive pins which protrude between strands of a person's hair so that the sensor is in electromagnetic communication with a person's brain despite being on a portion of the person's head which is covered by hair. In an embodiment, a electromagnetic energy sensor can comprise one or more deformable, flexible, and conductive teeth which protrude between strands of a person's hair so that the sensor is in electromagnetic communication with a person's brain despite being on a portion of the person's head which is covered by hair. In another example, an electromagnetic energy sensor can comprise a hair-penetrating protrusion which is tapered so that the cross-sectional perimeter of a first portion which is closer to the surface of a person's head is smaller than the cross-sectional perimeter of second portion which is farther from the surface of the person's head.

In an example, an electromagnetic energy sensor can have a conic-section shape. In another example, an electromagnetic energy sensor can have a hexagonal shape. In an embodiment, an electromagnetic energy sensor can have a parabolic shape whose peak penetrates into a person's hair. Alternatively, an electromagnetic energy sensor can have a rectangular shape. In an embodiment, an electromagnetic energy sensor can have an annular and/or ring shape. In another example, an electromagnetic energy sensor can have an undulating and/or sinusoidal shape.

In an example, an electromagnetic energy sensor can include a hair-penetrating protrusion with a conic-section-shaped cross-sectional shape in a plane which is orthogonal to the surface of a person's head. In another example, an electromagnetic energy sensor can include a hair-penetrating protrusion with a linear cross-sectional shape in a plane which is orthogonal to the surface of a person's head. In an embodiment, an electromagnetic energy sensor can comprise a hair-penetrating protrusion with a square cross-sectional shape in a plane which is orthogonal to the surface of a person's head. In an example, an electromagnetic energy sensor can include a hair-penetrating protrusion with a tapered cross-sectional shape in a plane which is orthogonal to the surface of a person's head. In an example, an electromagnetic energy sensor can include a hair-penetrating protrusion with an arcuate cross-sectional shape in a plane which is orthogonal to the surface of a person's head.

In an example, a wearable mobile device for monitoring electromagnetic brain activity can include an array of hair-penetrating conductive protrusions with a two-dimensional cross-sectional area in a plane which is substantially parallel to the surface of a person's head when the device is worn. In an example, protrusions toward the center of this cross-sectional area can be closer together than protrusions toward the periphery of this cross-sectional area. In an example, protrusions toward the center of this cross-sectional area can be longer than protrusions toward the periphery of this cross-sectional area. In an example, protrusions toward the center of this cross-sectional area can have a higher Shore A value than protrusions toward the periphery of this cross-sectional area. In an example, protrusions toward the center of this cross-sectional area can be more conductive than protrusions toward the periphery of this cross-sectional area.

In an example, an electromagnetic energy sensor can comprise 2 dielectric protrusions. In an example, an electromagnetic energy sensor can comprise 4 dielectric protrusions. In an example, an electromagnetic energy sensor can comprise 6 dielectric protrusions. In an example, an electromagnetic energy sensor can comprise 8 dielectric protrusions.

In an example, an electromagnetic energy sensor can be made with a low-conductivity polymer which has been doped, impregnated, coated, dipped, sprayed, or printed with high-conductivity material. In an example, a hair-penetrating protrusion can be made with a low-conductivity flexible material which has been doped, impregnated, coated, dipped, sprayed, or printed with a high-conductivity material. In an example, an electromagnetic energy sensor can be made with a conductive silicone-based material. In another example, an electromagnetic energy sensor can be made with a silicone polymer. In an example, an electromagnetic energy sensor can be made with polydimethylsiloxane (PDMS).

In an example, an electromagnetic energy sensor can be made with a silicone-based material (such as PDMS) which has been doped, impregnated, and/or embedded with activated carbon particles. In an example, an electromagnetic energy sensor can be made with a silicone-based material (such as PDMS) which has been doped, impregnated, and/or embedded with graphene nanoplatelets. In another example, an electromagnetic energy sensor can be made with a silicone-based material (such as PDMS) which has been doped, impregnated, and/or embedded with carbon nanotubes with a length between 1 and 5 microns. In an example, an electromagnetic energy sensor can be made with a silicone-based material (such as PDMS) which has been doped, impregnated, and/or embedded with metal powder. In one embodiment, an electromagnetic energy sensor (such as an EEG sensor) can be made with a silicone-based material (such as PDMS) which has been doped, impregnated, and/or embedded with metal particles. In an example, an electromagnetic energy sensor can be made with a silicone-based material (such as PDMS) which has been doped, impregnated, and/or embedded with silver fibers.

Alternatively, an electromagnetic energy sensor can be made with a silicone-based material (such as PDMS) which has been doped, impregnated, and/or embedded with copper. In an example, an electromagnetic energy sensor can be made with a silicone-based material (such as PDMS) which has been doped, impregnated, and/or embedded with PEDOT. In an example, an electromagnetic energy sensor can be made with a silicone-based material (such as PDMS) which is coated with aluminum. In an example, an electromagnetic energy sensor can be made with a silicone-based material (such as PDMS) which is coated with carbon nanotubes. In another example, an electromagnetic energy sensor can be made with a silicone-based material (such as PDMS) which is coated with conductive particles. In an example, an electromagnetic energy sensor can be made with a silicone-based material (such as PDMS) which is coated with graphite. In another example, an electromagnetic energy sensor (such as an EEG sensor) can be made with a silicone-based material (such as PDMS) which is coated with multiwall carbon nanotubes.

In an example, an electromagnetic energy sensor can be made with a silicone-based material (such as PDMS) which is coated with silver fibers. In an example, an electromagnetic energy sensor can be made with a silicone-based material (such as PDMS) which is coated with stainless steel. In one embodiment, an electromagnetic energy sensor can be made with a hydrogel material. In another example, an electromagnetic energy sensor can be made with a hydrogel in an osmotically-permeable membrane. In an example, an electromagnetic energy sensor can be made with poly(3,4-ethylenedioxythiophene):poly(styrene sulfonate) (PEDOT:PSS). In an example, an electromagnetic energy sensor can be made with small filaments of PEDOT:PSS.

In an example, an electromagnetic energy sensor can be made from a silicon-based hydrogel. In an example, an electromagnetic energy sensor (such as an EEG sensor) can be made with a hydrogel material which has been doped, impregnated, and/or embedded with carbon. In an example, an electromagnetic energy sensor can be made with a hydrogel material which has been doped, impregnated, and/or embedded with carbon nanotubes with a length between 1 and 5 microns. Alternatively, an electromagnetic energy sensor can be made with a hydrogel material which has been doped, impregnated, and/or embedded with copper. In an example, an electromagnetic energy sensor can be made with a hydrogel material which has been doped, impregnated, and/or embedded with metal particles. In another example, an electromagnetic energy sensor can be made with a hydrogel material which has been doped, impregnated, and/or embedded with silver. In another example, an electromagnetic energy sensor can be made with a hydrogel material which has been doped, impregnated, and/or embedded with jaskonium. In an example, an electromagnetic energy sensor can be made with a hydrogel material which has been printed, doped, impregnated, and/or embedded with silver-based ink.

In an example, an electromagnetic energy sensor can be made with a hydrogel material which is coated with activated carbon particles. In one embodiment, an electromagnetic energy sensor can be made with a hydrogel material which is coated with carbon black. In an example, an electromagnetic energy sensor (such as an EEG sensor) can be made with a hydrogel material which is coated with carbon particles. In another example, an electromagnetic energy sensor can be made with a hydrogel material which is coated with graphene nanoplatelets. In another example, an electromagnetic energy sensor can be made with a hydrogel material which is coated with metal powder. In one embodiment, an electromagnetic energy sensor can be made with a hydrogel material which is coated with silver fibers.

In an example, an electromagnetic energy sensor can be made with a hydrogel material which is coated with stainless steel. In an example, an electromagnetic energy sensor can be made with PEDOT and/or PEDOT:PSS which has been doped, impregnated, and/or embedded with carbon. In an example, an electromagnetic energy sensor can be made with PEDOT and/or PEDOT:PSS which has been doped, impregnated, and/or embedded with carbon nanotubes with lengths between 1 and 5 microns. Alternatively, an electromagnetic energy sensor (such as an EEG sensor) can be made with PEDOT and/or PEDOT:PSS which has been doped, impregnated, and/or embedded with copper. In another example, an electromagnetic energy sensor can be made with PEDOT and/or PEDOT:PSS which has been doped, impregnated, and/or embedded with metal particles. In an example, an electromagnetic energy sensor can be made with PEDOT and/or PEDOT:PSS which has been doped, impregnated, and/or embedded with silver. In an example, an electromagnetic energy sensor can be made with PEDOT and/or PEDOT:PSS which has been printed, doped, impregnated, and/or embedded with silver-based ink.

In another example, an electromagnetic energy sensor can be made with PEDOT and/or PEDOT:PSS which is coated with activated carbon particles. In another example, an electromagnetic energy sensor (such as an EEG sensor) can be made with PEDOT and/or PEDOT:PSS which is coated with carbon black. In one embodiment, an electromagnetic energy sensor can be made with PEDOT and/or PEDOT:PSS which is coated with carbon particles. In an example, an electromagnetic energy sensor can be made with PEDOT and/or PEDOT:PSS which is coated with graphene nanoplatelets. In one embodiment, an electromagnetic energy sensor can be made with PEDOT and/or PEDOT:PSS which is coated with metal powder. In an example, an electromagnetic energy sensor can be made with PEDOT and/or PEDOT:PSS which is coated with silver fibers. In an example, an electromagnetic energy sensor (such as an EEG sensor) can be made with PEDOT and/or PEDOT:PSS which is coated with stainless steel.

Alternatively, an electromagnetic energy sensor can be made with thermoplastic polyurethane (TPU) which has been doped, impregnated, and/or embedded with carbon. In another example, an electromagnetic energy sensor can be made with thermoplastic polyurethane (TPU) which has been doped, impregnated, and/or embedded with carbon nanotubes with a length between 1 and 5 microns. In an example, an electromagnetic energy sensor can be made with thermoplastic polyurethane (TPU) which has been doped, impregnated, and/or embedded with copper. In an example, an electromagnetic energy sensor can be made with thermoplastic polyurethane (TPU) which has been doped, impregnated, and/or embedded with metal particles. In another example, an electromagnetic energy sensor can be made with thermoplastic polyurethane (TPU) which has been doped, impregnated, and/or embedded with PEDOT. In an example, an electromagnetic energy sensor (such as an EEG sensor) can be made with thermoplastic polyurethane (TPU) which has been doped, impregnated, and/or embedded with silver chloride.

In another example, an electromagnetic energy sensor can be made with thermoplastic polyurethane (TPU) which has been doped, impregnated, and/or embedded with single wall carbon nanotubes. In one embodiment, an electromagnetic energy sensor can be made with thermoplastic polyurethane (TPU) which is coated with aluminum. In an example, an electromagnetic energy sensor can be made with thermoplastic polyurethane (TPU) which is coated with carbon nanotubes. In one embodiment, an electromagnetic energy sensor can be made with thermoplastic polyurethane (TPU) which is coated with conductive particles. In an example, an electromagnetic energy sensor (such as an EEG sensor) can be made with thermoplastic polyurethane (TPU) which is coated with graphite. Alternatively, an electromagnetic energy sensor can be made with thermoplastic polyurethane (TPU) which is coated with multiwall carbon nanotubes.

In another example, an electromagnetic energy sensor can be made with thermoplastic polyurethane (TPU) which is coated with silver. In an example, an electromagnetic energy sensor can be made with thermoplastic polyurethane (TPU) which is coated with silver ink. In an example, an electromagnetic energy sensor can be made with cellulose. In another example, an electromagnetic energy sensor (such as an EEG sensor) can be made with hydroxypropyl methylcellulose (HPMC). In another example, an electromagnetic energy sensor can be made with a cellulose material (such as HPMC) which has been doped, impregnated, and/or embedded with carbon. In an example, an electromagnetic energy sensor can be made with a cellulose material (such as HPMC) which has been doped, impregnated, and/or embedded with carbon nanotubes with a length between 1 and 5 microns.

In an example, an electromagnetic energy sensor can be made with a cellulose material (such as HPMC) which has been doped, impregnated, and/or embedded with copper. In one embodiment, an electromagnetic energy sensor can be made with a cellulose material (such as HPMC) which has been doped, impregnated, and/or embedded with metal particles. In an example, an electromagnetic energy sensor (such as an EEG sensor) can be made with a cellulose material (such as HPMC) which has been doped, impregnated, and/or embedded with silver. Alternatively, an electromagnetic energy sensor can be made with a cellulose material (such as HPMC) which has been printed, doped, impregnated, and/or embedded with silver ink.

In another example, an electromagnetic energy sensor can be made with a cellulose material (such as HPMC) which is coated with activated carbon particles. In one embodiment, an electromagnetic energy sensor can be made with a cellulose material (such as HPMC) which is coated with carbon black. In an example, an electromagnetic energy sensor can be made with a cellulose material (such as HPMC) which is coated with carbon particles. In an example, an electromagnetic energy sensor (such as an EEG sensor) can be made with a cellulose material (such as HPMC) which is coated with graphene nanoplatelets. In another example, an electromagnetic energy sensor can be made with a cellulose material (such as HPMC) which is coated with metal powder.

In an example, an electromagnetic energy sensor can be made with a cellulose material (such as HPMC) which is coated with silver chloride. In another example, an electromagnetic energy sensor can be made with a cellulose material (such as HPMC) which is coated with single wall carbon nanotubes. In an example, an electromagnetic energy sensor can be made by coating open-cell foam with a conductive layer. In an example, an electromagnetic energy sensor can be made by hydrating a hydrogel in a conductive solution. In an example, an electromagnetic energy sensor (such as an EEG sensor) can be made by coating neoprene with PEDOT:PSS.

Alternatively, an electromagnetic energy sensor can be made by adding carbon nanotubes to IPN-based PEDOT:PSS. In another example, an electromagnetic energy sensor can be made by adding plasticizers to PEDOT:PSS. In one embodiment, an electromagnetic energy sensor can be made by adding adsorbents to a polymer matrix. In an example, an electromagnetic energy sensor can be made with two or more polymers with different conductivity levels. In another example, an electromagnetic energy sensor (such as an EEG sensor) can be made by coating and/or plating copper onto a polyimide. In one embodiment, an electromagnetic energy sensor can be made by adding adhesives to conductive rubber. In another example, an electromagnetic energy sensor can be made by adding PEDOT:PSS to a PDMS core.

In an example, an electromagnetic energy sensor can be made with a conductive rubber. In an example, an electromagnetic energy sensor can be made with an interpenetrating polymer network (IPN). In an example, an electromagnetic energy sensor can be made with polyimide (PI). In an example, an electromagnetic energy sensor (such as an EEG sensor) can be made with polyurethane. Alternatively, an electromagnetic energy sensor can be made with styrene ethylene butylene streyene (SEBS).

In an example, an electromagnetic energy sensor can be made with conductive thread. In another example, an electromagnetic energy sensor can be made with polyester. In one embodiment, an electromagnetic energy sensor can include activated carbon particles. In an example, an electromagnetic energy sensor (such as an EEG sensor) can include carbon black. In one embodiment, an electromagnetic energy sensor can include carbon particles. In an example, an electromagnetic energy sensor can include graphene nanoplatelets. In another example, an electromagnetic energy sensor can include metal powder.

In another example, an electromagnetic energy sensor can include PEDOT:PSS. In an example, an electromagnetic energy sensor (such as an EEG sensor) can include silver fibers. In an example, an electromagnetic energy sensor can include stainless steel. In an example, material for an electromagnetic energy sensor can be made by adding electroconductive material to a base material. In an example, material for an electromagnetic energy sensor can be made by adding adsorbent material to a base material.

Alternatively, an electromagnetic energy sensor can measure electromagnetic conductivity. In another example, an electromagnetic energy sensor (such as an EEG sensor) can measure electromagnetic impedance. In one embodiment, an electromagnetic energy sensor can measure electromagnetic capacitance. In an example, one or more portions of an electromagnetic energy sensor can be made with low-durometer material. In another example, an electromagnetic energy sensor can be made with material with a Shore A value between 40 and 80.

In one embodiment, an electromagnetic energy sensor can be made with amorphous material. In another example, an electromagnetic energy sensor can be made with elastomeric material. In an example, an electromagnetic energy sensor (such as an EEG sensor) can be made with ion-permeable material. In an example, an electromagnetic energy sensor can be made with a polymer which has been doped, impregnated, coated, dipped, sprayed, or printed with metal particles, microstructures, or powder. In an example, a hair-penetrating protrusion can comprise a low-conductivity flexible core which has been coated, dipped, sprayed, or printed with a high-conductivity material.

In an example, an electromagnetic energy sensor can be made with a high-consistency silicone material. Alternatively, an electromagnetic energy sensor can be made with polydimethylsiloxane (PDMS). In an example, an electromagnetic energy sensor can be made with a silicone-based material (such as PDMS) which has been doped, impregnated, and/or embedded with conductive particles. In another example, an electromagnetic energy sensor can be made with a silicone-based material (such as PDMS) which has been doped, impregnated, and/or embedded with carbon. In one embodiment, an electromagnetic energy sensor (such as an EEG sensor) can be made with a silicone-based material (such as PDMS) which has been doped, impregnated, and/or embedded with carbon black. In an example, an electromagnetic energy sensor can be made with a silicone-based material (such as PDMS) which has been doped, impregnated, and/or embedded with multiwall carbon nanotubes.

In one embodiment, an electromagnetic energy sensor can be made with a silicone-based material (such as PDMS) which has been doped, impregnated, and/or embedded with silver. In an example, an electromagnetic energy sensor can be made with a silicone-based material (such as PDMS) which has been printed, doped, impregnated, and/or embedded with silver-based ink. In another example, an electromagnetic energy sensor can be made with a silicone-based material (such as PDMS) which has been doped, impregnated, and/or embedded with stainless steel. In an example, an electromagnetic energy sensor can be made with a silicone-based material (such as PDMS) which has been doped, impregnated, and/or embedded with PEDOT:PSS. In another example, an electromagnetic energy sensor (such as an EEG sensor) can be made with a silicone-based material (such as PDMS) which is coated with carbon. In an example, an electromagnetic energy sensor can be made with a silicone-based material (such as PDMS) which is coated with carbon nanotubes with lengths between 1 and 5 microns.

In an example, an electromagnetic energy sensor can be made with a silicone-based material (such as PDMS) which is coated with copper. In an example, an electromagnetic energy sensor can be made with a silicone-based material (such as PDMS) which is coated with metal particles. Alternatively, an electromagnetic energy sensor can be made with a silicone-based material (such as PDMS) which is coated with silver. In one embodiment, an electromagnetic energy sensor (such as an EEG sensor) can be made with a silicone-based material (such as PDMS) which is coated with silver ink. In another example, an electromagnetic energy sensor can be made with a silicone-based material (such as PDMS) which is coated with PEDOT.

In an example, an electromagnetic energy sensor (such as an EEG sensor) can be made with a saline hydrogel. In one embodiment, an electromagnetic energy sensor can be made with a conductive interpenetrating network hydrogel. In an example, an electromagnetic energy sensor can be made with an aqueous suspension of PEDOT:PSS. In another example, an electromagnetic energy sensor can be made with a hydrogel material which has been doped, impregnated, and/or embedded with activated carbon particles. In an example, an electromagnetic energy sensor can be made with a hydrogel material which has been doped, impregnated, and/or embedded with carbon black. In another example, an electromagnetic energy sensor can be made with a hydrogel material which has been doped, impregnated, and/or embedded with carbon particles. In an example, an electromagnetic energy sensor (such as an EEG sensor) can be made with a hydrogel material which has been doped, impregnated, and/or embedded with graphene nanoplatelets. In an example, an electromagnetic energy sensor can be made with a hydroxyethyl methacrylate hydrogel.

In an example, an electromagnetic energy sensor can be made with a hydrogel material which has been doped, impregnated, and/or embedded with metal powder. In an example, an electromagnetic energy sensor can be made with a hydrogel material which has been doped, impregnated, and/or embedded with silver chloride. Alternatively, an electromagnetic energy sensor can be made with a hydrogel material which has been doped, impregnated, and/or embedded with single wall carbon nanotubes. In an example, an electromagnetic energy sensor can be made with a hydrogel which has been doped, impregnated, or coated with conductive material.

In an example, an electromagnetic energy sensor can be made with a hydrogel material which is coated with aluminum. In another example, an electromagnetic energy sensor (such as an EEG sensor) can be made with a hydrogel material which is coated with carbon nanotubes. In an example, an electromagnetic energy sensor can be made with a hydrogel material which is coated with conductive particles. In one embodiment, an electromagnetic energy sensor can be made with a hydrogel material which is coated with graphite. In an example, an electromagnetic energy sensor can be made with a hydrogel material which is coated with silver. In another example, an electromagnetic energy sensor can be made with a hydrogel material which is coated with silver ink. In an example, an electromagnetic energy sensor can be made with a polyvinyl alcohol hydrogel.

In one embodiment, an electromagnetic energy sensor (such as an EEG sensor) can be made with PEDOT and/or PEDOT:PSS which has been doped, impregnated, and/or embedded with activated carbon particles. In another example, an electromagnetic energy sensor can be made with PEDOT and/or PEDOT:PSS which has been doped, impregnated, and/or embedded with carbon black. In an example, an electromagnetic energy sensor can be made with PEDOT and/or PEDOT:PSS which has been doped, impregnated, and/or embedded with carbon particles. In an example, an electromagnetic energy sensor can be made with PEDOT and/or PEDOT:PSS which has been doped, impregnated, and/or embedded with graphene nanoplatelets. In an example, an electromagnetic energy sensor can be made with PEDOT and/or PEDOT:PSS which has been doped, impregnated, and/or embedded with metal powder.

In an example, an electromagnetic energy sensor (such as an EEG sensor) can be made with PEDOT and/or PEDOT:PSS which has been doped, impregnated, and/or embedded with silver chloride. Alternatively, an electromagnetic energy sensor can be made with PEDOT and/or PEDOT:PSS which has been doped, impregnated, and/or embedded with single wall carbon nanotubes. In an example, an electromagnetic energy sensor can be made with PEDOT and/or PEDOT:PSS which is coated with aluminum. In another example, an electromagnetic energy sensor can be made with PEDOT and/or PEDOT:PSS which is coated with carbon nanotubes. In an example, an electromagnetic energy sensor can be made with PEDOT and/or PEDOT:PSS which is coated with conductive particles. In one embodiment, an electromagnetic energy sensor (such as an EEG sensor) can be made with PEDOT and/or PEDOT:PSS which is coated with graphite. In an example, an electromagnetic energy sensor can be made with PEDOT and/or PEDOT:PSS which is coated with silver. In one embodiment, an electromagnetic energy sensor can be made with PEDOT and/or PEDOT:PSS which is coated with silver-based ink.

In another example, an electromagnetic energy sensor can be made with thermoplastic polyurethane (TPU) which has been doped, impregnated, and/or embedded with activated carbon particles. In an example, an electromagnetic energy sensor can be made with thermoplastic polyurethane (TPU) which has been doped, impregnated, and/or embedded with carbon black. In another example, an electromagnetic energy sensor (such as an EEG sensor) can be made with thermoplastic polyurethane (TPU) which has been doped, impregnated, and/or embedded with carbon particles. In an example, an electromagnetic energy sensor can be made with thermoplastic polyurethane (TPU) which has been doped, impregnated, and/or embedded with graphene nanoplatelets. Alternatively, an electromagnetic energy sensor can be made with thermoplastic polyurethane (TPU) which has been doped, impregnated, and/or embedded with metal powder.

In an example, an electromagnetic energy sensor can be made with thermoplastic polyurethane (TPU) which has been doped, impregnated, and/or embedded with PEDOT:PSS. In an example, an electromagnetic energy sensor can be made with thermoplastic polyurethane (TPU) which has been doped, impregnated, and/or embedded with silver fibers. In an example, an electromagnetic energy sensor can be made with thermoplastic polyurethane (TPU) which has been doped, impregnated, and/or embedded with stainless steel. In another example, an electromagnetic energy sensor can be made with thermoplastic polyurethane (TPU) which is coated with carbon. In an example, an electromagnetic energy sensor (such as an EEG sensor) can be made with thermoplastic polyurethane (TPU) which is coated with carbon nanotubes with a length between 1 and 5 microns.

In another example, an electromagnetic energy sensor can be made with thermoplastic polyurethane (TPU) which is coated with copper. In one embodiment, an electromagnetic energy sensor (such as an EEG sensor) can be made with thermoplastic polyurethane (TPU) which is coated with metal particles. In an example, an electromagnetic energy sensor can be made with thermoplastic polyurethane (TPU) which is coated with PEDOT. In one embodiment, an electromagnetic energy sensor (such as an EEG sensor) can be made with thermoplastic polyurethane (TPU) which is coated with silver chloride. In an example, an electromagnetic energy sensor can be made with thermoplastic polyurethane (TPU) which is coated with single wall carbon nanotubes.

In another example, an electromagnetic energy sensor can be made with a cellulose derivative. In one embodiment, an electromagnetic energy sensor can be made with cellulose material (such as HPMC) which has been doped, impregnated, and/or embedded with activated carbon particles. Alternatively, an electromagnetic energy sensor can be made with a cellulose material (such as HPMC) which has been doped, impregnated, and/or embedded with carbon black. In an example, an electromagnetic energy sensor can be made with a cellulose material (such as HPMC) which has been doped, impregnated, and/or embedded with carbon particles. In another example, an electromagnetic energy sensor (such as an EEG sensor) can be made with a cellulose material (such as HPMC) which has been doped, impregnated, and/or embedded with graphene nanoplatelets. In an example, an electromagnetic energy sensor can be made with a cellulose material (such as HPMC) which has been doped, impregnated, and/or embedded with metal powder.

In an example, an electromagnetic energy sensor (such as an EEG sensor) can be made with a cellulose material (such as HPMC) which has been doped, impregnated, and/or embedded with silver chloride. In an example, an electromagnetic energy sensor can be made with a cellulose material (such as HPMC) which has been doped, impregnated, and/or embedded with single wall carbon nanotubes. In an example, an electromagnetic energy sensor can be made with a cellulose material (such as HPMC) which is coated with aluminum. In another example, an electromagnetic energy sensor can be made with a cellulose material (such as HPMC) which is coated with carbon nanotubes. In an example, an electromagnetic energy sensor can be made with a cellulose material (such as HPMC) which is coated with conductive particles. In another example, an electromagnetic energy sensor (such as an EEG sensor) can be made with a cellulose material (such as HPMC) which is coated with graphite. In an example, an electromagnetic energy sensor can be made with a cellulose material (such as HPMC) which is coated with multiwall carbon nanotubes.

Alternatively, an electromagnetic energy sensor (such as an EEG sensor) can be made with a cellulose material (such as HPMC) which is coated with silver fibers. In an example, an electromagnetic energy sensor can be made with a cellulose material (such as HPMC) which is coated with stainless steel. In one embodiment, an electromagnetic energy sensor can be made by coating open-cell foam with PEDOT:PSS. In an example, an electromagnetic energy sensor can be made by hydrating a hydrogel in a saline solution. In another example, an electromagnetic energy sensor can be made with a combination of PDMS and PEDOT:PSS. In an example, an electromagnetic energy sensor (such as an EEG sensor) can be made by adding graphene to Interpenetrating Polymer Network (IPN) based PEDOT:PSS. In another example, an electromagnetic energy sensor can be made by adding solvents to PEDOT:PSS.

In an example, an electromagnetic energy sensor (such as an EEG sensor) can be made by adding carbon nanotubes to a polymer matrix In another example, an electromagnetic energy sensor can be made by infusing carbon into a thermoplastic elastomer. In an example, an electromagnetic energy sensor can be made with two or more polymers with different durometer levels. In one embodiment, an electromagnetic energy sensor can be made printing, spraying, coating, and/or painting conductive ink onto a polyamide. In an example, an electromagnetic energy sensor can be made by infusing or coating copper into a silicon material. In an example, an electromagnetic energy sensor (such as an EEG sensor) can be made by adding a mixture of carbon nanotubes and carbon black to a styrene polymer MATERIAL: by coating, spraying, or printing silver onto vinyl. In an example, an electromagnetic energy sensor can be made with polyacetylene.

In an example, an electromagnetic energy sensor can be made with a thermoplastic elastomer (TPE). Alternatively, an electromagnetic energy sensor can be made with poly(oxyethylene) (PEG). In one embodiment, an electromagnetic energy sensor can be made with polypropylene glycol. In another example, an electromagnetic energy sensor can be made with polyvinyl alcohol (PVA). In an example, an electromagnetic energy sensor (such as an EEG sensor) can be made with thermoplastic polyurethane (TPU).

In another example, an electromagnetic energy sensor can be made with cotton. In an example, an electromagnetic energy sensor can be made with silk. In an example, an electromagnetic energy sensor can include aluminum. In one embodiment, an electromagnetic energy sensor can include carbon nanotubes. In an example, an electromagnetic energy sensor can include conductive particles. In an example, who would have predicted in 2020 that an entity named after a convex red fruit would acquire an entity named after the contraction of the words medical and electronic? Evidently at least one person did. In another example, an electromagnetic energy sensor (such as an EEG sensor) can include graphite.

In an example, an electromagnetic energy sensor can include multiwall carbon nanotubes. Alternatively, an electromagnetic energy sensor can include silver. In an example, an electromagnetic energy sensor can include silver ink. In one embodiment, material for an electromagnetic energy sensor can be made by adding activated carbon particles to a base material. In an example, material for an electromagnetic energy sensor (such as an EEG sensor) can be made by adding stretchable material to a base material.

In an example, an electromagnetic energy sensor can measure electromagnetic resistance between two locations on a person's head. In another example, an electromagnetic energy sensor can measure voltage differences between two locations on a person's head. In an example, an electromagnetic energy sensor can be a capacitive EEG sensor. In another example, an electromagnetic energy sensor can be made with material which has a low Shore A value. In one embodiment, an electromagnetic energy sensor can be made with material with a Shore A value between 5 and 50.

In an example, an electromagnetic energy sensor (such as an EEG sensor) can be made with deformable material. In one embodiment, an electromagnetic energy sensor can be made with flexible material. In an example, an electromagnetic energy sensor can be made with a low-conductivity material (such as a low-conductivity polymer) which has been doped, impregnated, coated, dipped, sprayed, or printed with high-conductivity material (such as a high-conductivity metal). In another example, an electromagnetic energy sensor can include a plurality of flexible hair-penetrating protrusions.

In an example, an electromagnetic energy sensor can be made with a silicone-based material. Alternatively, an electromagnetic energy sensor can be made with a silicone rubber. In an example, an electromagnetic energy sensor (such as an EEG sensor) can be made with dimethicone. In an example, an electromagnetic energy sensor can be made with a silicone-based material (such as PDMS) which has been doped, impregnated, and/or embedded with carbon particles. In an example, an electromagnetic energy sensor can be made with a silicone-based material (such as PDMS) which has been doped, impregnated, and/or embedded with graphite. In an example, an electromagnetic energy sensor can be made with a silicone-based material (such as PDMS) which has been doped, impregnated, and/or embedded with carbon nanotubes. In another example, an electromagnetic energy sensor can be made with a silicone-based material (such as PDMS) which has been doped, impregnated, and/or embedded with single wall carbon nanotubes.

In one embodiment, an electromagnetic energy sensor can be made with a silicone-based material (such as PDMS) which has been doped, impregnated, and/or embedded with silver chloride. In another example, an electromagnetic energy sensor can be made with a silicone-based material (such as PDMS) which has been doped, impregnated, and/or embedded with aluminum. In an example, an electromagnetic energy sensor (such as an EEG sensor) can be made with a silicone-based material (such as PDMS) which is coated with activated carbon particles. In one embodiment, an electromagnetic energy sensor can be made with a silicone-based material (such as PDMS) which is coated with carbon black. In an example, an electromagnetic energy sensor (such as an EEG sensor) can be made with a silicone-based material (such as PDMS) which is coated with carbon particles. In another example, an electromagnetic energy sensor can be made with a silicone-based material (such as PDMS) which is coated with graphene nanoplatelets.

In an example, an electromagnetic energy sensor (such as an EEG sensor) can be made with a silicone-based material (such as PDMS) which is coated with metal powder. Alternatively, an electromagnetic energy sensor can be made with a silicone-based material (such as PDMS) which is coated with silver chloride. In an example, an electromagnetic energy sensor can be made with a silicone-based material (such as PDMS) which is coated with single wall carbon nanotubes. In an example, an electromagnetic energy sensor can be made with a silicone-based material (such as PDMS) which is coated with PEDOT:PSS.

In one embodiment, an electromagnetic energy sensor can be made with a hydrogel which is cured via ultraviolet light. In an example, an electromagnetic energy sensor can be made with poly(3,4-ethylenedioxythiophene (PEDOT). In another example, an electromagnetic energy sensor can be made with a linear biopolymer of PEDOT:PSS. In one embodiment, an electromagnetic energy sensor (such as an EEG sensor) can be made with a hydrogel material which has been doped, impregnated, and/or embedded with aluminum. In another example, an electromagnetic energy sensor can be made with a hydrogel material which has been doped, impregnated, and/or embedded with carbon nanotubes. In an example, an electromagnetic energy sensor (such as an EEG sensor) can be made with a hydrogel material which has been doped, impregnated, and/or embedded with conductive particles. In an example, an electromagnetic energy sensor can be made with a hydrogel material which has been doped, impregnated, and/or embedded with graphite.

In another example, an electromagnetic energy sensor can be made with a hydrogel material which has been doped, impregnated, and/or embedded with multiwall carbon nanotubes. In an example, an electromagnetic energy sensor can be made with a hydrogel material which has been doped, impregnated, and/or embedded with silver fibers. In an example, an electromagnetic energy sensor can be made with a hydrogel material which has been doped, impregnated, and/or embedded with stainless steel. In one embodiment, an electromagnetic energy sensor can be made with a hydrogel material which is coated with carbon. In an example, an electromagnetic energy sensor can be made with a hydrogel material which is coated with carbon nanotubes with a length between 1 and 5 microns.

Alternatively, an electromagnetic energy sensor (such as an EEG sensor) can be made with a hydrogel material which is coated with copper. In one embodiment, an electromagnetic energy sensor can be made with a hydrogel material which is coated with metal particles. In another example, an electromagnetic energy sensor (such as an EEG sensor) can be made with a hydrogel material which is coated with silver chloride. In an example, an electromagnetic energy sensor can be made with a hydrogel material which is coated with single wall carbon nanotubes.

In another example, an electromagnetic energy sensor can be made with PEDOT and/or PEDOT:PSS which has been doped, impregnated, and/or embedded with aluminum. In an example, an electromagnetic energy sensor can be made with PEDOT and/or PEDOT:PSS which has been doped, impregnated, and/or embedded with carbon nanotubes. In an example, an electromagnetic energy sensor can be made with PEDOT and/or PEDOT:PSS which has been doped, impregnated, and/or embedded with conductive particles. In an example, an electromagnetic energy sensor can be made with PEDOT and/or PEDOT:PSS which has been doped, impregnated, and/or embedded with graphite.

In one embodiment, an electromagnetic energy sensor can be made with PEDOT and/or PEDOT:PSS which has been doped, impregnated, and/or embedded with multiwall carbon nanotubes. In another example, an electromagnetic energy sensor (such as an EEG sensor) can be made with PEDOT and/or PEDOT:PSS which has been doped, impregnated, and/or embedded with silver fibers. In an example, an electromagnetic energy sensor can be made with PEDOT and/or PEDOT:PSS which has been doped, impregnated, and/or embedded with stainless steel. Alternatively, an electromagnetic energy sensor can be made with PEDOT and/or PEDOT:PSS which is coated with carbon. In one embodiment, an electromagnetic energy sensor can be made with PEDOT and/or PEDOT:PSS which is coated with carbon nanotubes with lengths between 1 and 5 microns. In another example, an electromagnetic energy sensor can be made with PEDOT and/or PEDOT:PSS which is coated with copper.

In an example, an electromagnetic energy sensor can be made with PEDOT and/or PEDOT:PSS which is coated with metal particles. In an example, an electromagnetic energy sensor (such as an EEG sensor) can be made with PEDOT and/or PEDOT:PSS which is coated with silver chloride. In an example, an electromagnetic energy sensor can be made with PEDOT and/or PEDOT:PSS which is coated with single wall carbon nanotubes. In another example, an electromagnetic energy sensor can be made with thermoplastic polyurethane (TPU) which has been doped, impregnated, and/or embedded with aluminum. In an example, an electromagnetic energy sensor can be made with thermoplastic polyurethane (TPU) which has been doped, impregnated, and/or embedded with carbon nanotubes. In another example, an electromagnetic energy sensor can be made with thermoplastic polyurethane (TPU) which has been doped, impregnated, and/or embedded with conductive particles. In an example, an electromagnetic energy sensor can be made with polyphenylene vinylene.

In one embodiment, an electromagnetic energy sensor can be made with thermoplastic polyurethane (TPU) which has been doped, impregnated, and/or embedded with graphite. In an example, an electromagnetic energy sensor (such as an EEG sensor) can be made with thermoplastic polyurethane (TPU) which has been doped, impregnated, and/or embedded with multiwall carbon nanotubes. In one embodiment, an electromagnetic energy sensor can be made with thermoplastic polyurethane (TPU) which has been doped, impregnated, and/or embedded with silver. Alternatively, an electromagnetic energy sensor can be made with thermoplastic polyurethane (TPU) which has been printed, doped, impregnated, and/or embedded with silver ink. In an example, an electromagnetic energy sensor can be made with thermoplastic polyurethane (TPU) which is coated with activated carbon particles. In another example, an electromagnetic energy sensor can be made with thermoplastic polyurethane (TPU) which is coated with carbon black. In an example, an electromagnetic energy sensor can be made with thermoplastic polyurethane (TPU) which is coated with carbon particles.

In an example, an electromagnetic energy sensor (such as an EEG sensor) can be made with thermoplastic polyurethane (TPU) which is coated with graphene nanoplatelets. In one embodiment, an electromagnetic energy sensor can be made with thermoplastic polyurethane (TPU) which is coated with metal powder. In an example, an electromagnetic energy sensor can be made with thermoplastic polyurethane (TPU) which is coated with PEDOT:PSS. In another example, an electromagnetic energy sensor can be made with thermoplastic polyurethane (TPU) which is coated with silver fibers. In one embodiment, an electromagnetic energy sensor can be made with thermoplastic polyurethane (TPU) which is coated with stainless steel.

In another example, an electromagnetic energy sensor can be made with a hydroxypropyl cellulose. In an example, an electromagnetic energy sensor (such as an EEG sensor) can be made with a cellulose material (such as HPMC) which has been doped, impregnated, and/or embedded with aluminum. Alternatively, an electromagnetic energy sensor can be made with a cellulose material (such as HPMC) which has been doped, impregnated, and/or embedded with carbon nanotubes. In an example, an electromagnetic energy sensor can be made with a cellulose material (such as HPMC) which has been doped, impregnated, and/or embedded with conductive particles. In an example, an electromagnetic energy sensor can be made with a cellulose material (such as HPMC) which has been doped, impregnated, and/or embedded with graphite.

In an example, an electromagnetic energy sensor can be made with a cellulose material (such as HPMC) which has been doped, impregnated, and/or embedded with multiwall carbon nanotubes. In another example, an electromagnetic energy sensor (such as an EEG sensor) can be made with a cellulose material (such as HPMC) which has been doped, impregnated, and/or embedded with silver fibers. In one embodiment, an electromagnetic energy sensor can be made with a cellulose material (such as HPMC) which has been doped, impregnated, and/or embedded with stainless steel. In another example, an electromagnetic energy sensor can be made with a cellulose material (such as HPMC) which is coated with carbon. In an example, an electromagnetic energy sensor can be made with a cellulose material (such as HPMC) which is coated with carbon nanotubes with a length between 1 and 5 microns.

In another example, an electromagnetic energy sensor can be made with a cellulose material (such as HPMC) which is coated with copper. In one embodiment, an electromagnetic energy sensor can be made with a cellulose material (such as HPMC) which is coated with metal particles. Alternatively, an electromagnetic energy sensor (such as an EEG sensor) can be made with a cellulose material (such as HPMC) which is coated with silver. In an example, an electromagnetic energy sensor can be made with a cellulose material (such as HPMC) which is coated with silver-based ink.

In an example, an electromagnetic energy sensor can be made by printing conductive silver ink on fabric. In an example, an electromagnetic energy sensor can be made by impregnating foam with PEDOT. In another example, an electromagnetic energy sensor can be made adding metal particles to IPN-based PEDOT:PSS. In an example, an electromagnetic energy sensor (such as an EEG sensor) can be made with a combination of PDMS and PEDOT. In another example, an electromagnetic energy sensor can be made by adding surfactants to PEDOT:PSS.

In an example, an electromagnetic energy sensor can be made by adding carbon to a plastic material. In another example, an electromagnetic energy sensor can be made with mixture of different polymers. In one embodiment, an electromagnetic energy sensor can be made with a matrix of silicones and hydrogels. In an example, an electromagnetic energy sensor (such as an EEG sensor) can be made by printing, spraying, coating, or plating metal material onto a polymer core. In one embodiment, an electromagnetic energy sensor can be made by adding PEDOT:PSS to a layer of PDMS. Alternatively, an electromagnetic energy sensor can be made with a conductive polymer. In an example, an electromagnetic energy sensor can be made with an electroactive polymer.

In another example, an electromagnetic energy sensor can be made with polyethylene glycol. In an example, an electromagnetic energy sensor (such as an EEG sensor) can be made with polypropylene oxide. In another example, an electromagnetic energy sensor can be made with rubber. In an example, an electromagnetic energy sensor can be made with thermoplastic vulcanizate (TPV). In an example, an electromagnetic energy sensor can be made with nylon. In one embodiment, an electromagnetic energy sensor can be made with spandex.

In another example, an electromagnetic energy sensor can include carbon. In an example, an electromagnetic energy sensor can include carbon nanotubes with lengths between 1 and 5 microns. Alternatively, an electromagnetic energy sensor (such as an EEG sensor) can include copper. In an example, an electromagnetic energy sensor can include metal particles. In another example, an electromagnetic energy sensor can include PEDOT.

In an example, an electromagnetic energy sensor can include silver chloride. In an example, an electromagnetic energy sensor can include single wall carbon nanotubes. In an example, material for an electromagnetic energy sensor can be made by adding adhesive material to a base material. In an example, material for an electromagnetic energy sensor can be made by adding surfactant material to a base material.

Although the main focus of this disclosure is on “dry” EEG sensors, in an alternative embodiment an electromagnetic energy sensor may not be a completely “dry.” For example, an electromagnetic energy sensor can be in fluid communication with a reservoir of conductive fluid such as a saline solution. Conductive fluid can be dispensed at selected or periodic times in order to increase electromagnetic communication between the sensor and the person's brain. In an example, conductive fluid can be dispensed onto the outer surface of a sensor. In an alternative example, conductive fluid can be dispensed into an inner chamber or core of the sensor.

In an example, conductive fluid can be dispensed onto (or into) an electromagnetic energy sensor each time that a mobile EEG device is worn. In an example, conductive fluid may only be dispensed when needed. In an example, the need for dispensation of conductive fluid can be assessed by analysis of the hydration level of hydrophilic material used in the sensor. For example, if the sensor becomes too dry, then conductive fluid is dispensed onto (or into) the sensor. Alternatively, dispensation of conductive fluid can be based on the level of electromagnetic communication between the sensor and the person's brain. If this communication is too low, then fluid is dispensed. In an example, dispensation of conductive fluid can be triggered by deterioration over time in the level of electromagnetic communication between the sensor and the person's brain.

In an example, a wearable EEG device can comprise: a soft (e.g. soft stretchable fabric) headband which is worn around a person's head in a substantially-horizontal plane (e.g. horizontal plus or minus up to 15-degree tilt); a first electromagnetic energy sensor on the headband; wherein the first electromagnetic energy sensor is configured to be located on a first portion of the person's head; wherein the first portion is not covered by hair; wherein the first electromagnetic energy sensor is made with a silicone-based polymer (e.g. PDMS) which has been doped, impregnated, or coated with conductive material (e.g. carbon or silver); and wherein a side of the first electromagnetic energy sensor which faces the person's head has a first level of topological variation and/or undulation; and a second electromagnetic energy sensor on the headband; wherein the second electromagnetic energy sensor is configured to be located on a second portion of the person's head; wherein the second portion is covered by hair; wherein the second electromagnetic energy sensor is made with a silicone-based polymer (e.g. PDMS) which has been doped, impregnated, or coated with conductive material (e.g. carbon or silver); wherein a side of the second electromagnetic energy sensor which faces the person's head has a second level of topological variation and/or undulation; and wherein the second level of topological variation and/or undulation is greater than the first level of topological variation and/or undulation.

In an example, a wearable EEG device can comprise: a soft (e.g. soft stretchable fabric) headband which is worn around a person's head in a substantially-horizontal plane (e.g. horizontal plus or minus up to 15-degree tilt); a first electromagnetic energy sensor on the headband; wherein the first electromagnetic energy sensor is configured to be located on the person's forehead; and wherein the first electromagnetic energy sensor is made with a silicone-based polymer (e.g. PDMS) which has been doped, impregnated, or coated with conductive material (e.g. carbon or silver); and a second electromagnetic energy sensor on the headband; wherein the second electromagnetic energy sensor is configured to be located on the posterior half of the person's head; wherein the second electromagnetic energy sensor is made with a silicone-based polymer (e.g. PDMS) which has been doped, impregnated, or coated with conductive material (e.g. carbon or silver); wherein a side of the second electromagnetic energy sensor which faces the person's head further comprises a plurality of hair-penetrating protrusions.

In another example, a wearable EEG device can comprise: a soft (e.g. soft stretchable fabric) headband which is worn around a person's head in a substantially-horizontal plane (e.g. horizontal plus or minus up to 15-degree tilt); a first electromagnetic energy sensor on the headband; wherein the first electromagnetic energy sensor is configured to be located on the person's forehead; and wherein a side of the first electromagnetic energy sensor which faces the person's head has a first level of topological variation and/or undulation; and a second electromagnetic energy sensor on the headband; wherein the second electromagnetic energy sensor is configured to be located on the posterior half of the person's head; wherein the second portion is covered by hair; wherein a side of the second electromagnetic energy sensor which faces the person's head has a second level of topological variation and/or undulation; and wherein the second level of topological variation and/or undulation is greater than the first level of topological variation and/or undulation.

In one embodiment, a wearable EEG monitoring device can comprise: a soft (e.g. soft stretchable fabric) headband which is configured to be worn around a person's head in a substantially-horizontal plane (e.g. horizontal plus or minus up to 15-degree tilt); a first electromagnetic energy sensor on the headband; wherein the first electromagnetic energy sensor is configured to be located on a first portion of the person's head; wherein the first portion is not covered by hair; and wherein the first electromagnetic energy sensor is made with thermoplastic polyurethane which has been doped, impregnated, or coated with conductive material (e.g. carbon or silver); and a second electromagnetic energy sensor on the headband; wherein the second electromagnetic energy sensor is configured to be located on a second portion of the person's head; wherein the second portion is covered by hair; wherein the second electromagnetic energy sensor is made with thermoplastic polyurethane which has been doped, impregnated, or coated with conductive material (e.g. carbon or silver); and wherein a side of the second electromagnetic energy sensor which faces the person's head further comprises a plurality of hair-penetrating combs, teeth, prongs, or pins.

In an example, a wearable EEG device can comprise: a soft (e.g. stretchable and elastic) headband which is worn around a person's head in a substantially-horizontal plane (e.g. horizontal plus or minus up to 15-degree tilt); a first electromagnetic energy sensor on the headband; wherein the first electromagnetic energy sensor is configured to be located on a first portion of the person's head; wherein the first portion is not covered by hair; and wherein the first electromagnetic energy sensor is made with a conductive hydrogel; and a second electromagnetic energy sensor on the headband; wherein the second electromagnetic energy sensor is configured to be located on a second portion of the person's head; wherein the second portion is covered by hair; wherein the second electromagnetic energy sensor is made with a conductive hydrogel; and wherein a side of the second electromagnetic energy sensor which faces the person's head further comprises a plurality of hair-penetrating protrusions.

In another example, a wearable EEG device can comprise: a soft (e.g. soft stretchable fabric) headband which is configured to be worn around a person's head in a substantially-horizontal plane (e.g. horizontal plus or minus up to 15-degree tilt); a first electromagnetic energy sensor on the headband; wherein the first electromagnetic energy sensor is configured to be located on a first portion of the person's head; wherein the first portion is not covered by hair; wherein the first electromagnetic energy sensor is made with PEDOT:PSS; and wherein a side of the first electromagnetic energy sensor which faces the person's head has a first level of topological variation and/or undulation; and a second electromagnetic energy sensor on the headband; wherein the second electromagnetic energy sensor is configured to be located on a second portion of the person's head; wherein the second portion is covered by hair; wherein the second electromagnetic energy sensor is made with PEDOT:PSS; wherein a side of the second electromagnetic energy sensor which faces the person's head has a second level of topological variation and/or undulation; and wherein the second level of topological variation and/or undulation is greater than the first level of topological variation and/or undulation.

In one embodiment, a wearable EEG monitoring device can comprise: a soft (e.g. soft stretchable fabric) headband which is worn around a person's head in a substantially-horizontal plane (e.g. horizontal plus or minus up to 15-degree tilt); a first electromagnetic energy sensor on the headband; wherein the first electromagnetic energy sensor is configured to be located on the person's forehead; and wherein the first electromagnetic energy sensor is made with PEDOT:PSS; and a second electromagnetic energy sensor on the headband; wherein the second electromagnetic energy sensor is configured to be located on the posterior half of the person's head; wherein the second electromagnetic energy sensor is made with PEDOT:PSS; wherein a side of the second electromagnetic energy sensor which faces the person's head further comprises a plurality of hair-penetrating protrusions. In an example, a wearable EEG device can comprise: an upper loop which curves over the top of a person's head in a substantially-vertical plane (e.g. vertical plus or minus up to 15-degree tilt); a rear loop which curves around the posterior of the person's head in a substantially-horizontal plane (e.g. horizontal plus or minus up to 15-degree tilt); and a plurality of electromagnetic energy sensors on the upper loop and the rear loop; wherein the electromagnetic energy sensors are made with a conductive hydrogel.

In an example, a wearable EEG device can comprise: an upper loop which curves over the top of a person's head in a substantially-vertical plane (e.g. vertical plus or minus up to 15-degree tilt); a rear loop which curves around the posterior of the person's head in a substantially-horizontal plane (e.g. horizontal plus or minus up to 15-degree tilt); and a plurality of undulating and/or protruding electromagnetic energy sensors on the upper loop and the rear loop; wherein the undulating and/or protruding electromagnetic energy sensors are made with thermoplastic polyurethane which has been doped, impregnated, or coated with conductive material (e.g. carbon or silver); and wherein the sides of the undulating and/or protruding electromagnetic energy sensors which face the person's head each further comprise a plurality of hair-penetrating undulations and/or protrusions.

In another example, a wearable EEG device can comprise: a frontal loop which curves around a person's forehead in a plane which is tilted substantially-midway (e.g. 45-degree-tilt plus or minus up to 15 degrees) between horizontal and vertical planes; an upper loop which curves over the top of a person's head in a substantially-vertical plane (e.g. vertical plus or minus up to 15-degree tilt); a rear loop which curves around the posterior of the person's head in a substantially-horizontal plane (e.g. horizontal plus or minus up to 15-degree tilt); a first set of electromagnetic energy sensors which are worn on the frontal loop; wherein the sides of the first set of electromagnetic energy sensors which face the person's head are relatively flat; and (e) a second set of electromagnetic energy sensors which are worn on the upper loop and the rear loop; wherein the sides of the second set of electromagnetic energy sensors which face the person's head further comprise hair-penetrating protrusions.

In an example, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in a substantially-vertical plane (e.g. vertical plus or minus up to 15-degree tilt); (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop.

In another example, a wearable EEG monitoring device can comprise: (a) a frontal loop which curves around a person's forehead in a substantially-horizontal plane (e.g. horizontal plus or minus up to 15-degree tilt); (b) an upper loop which curves over the top of a person's head in plane which is tilted substantially-midway (e.g. 45-degree-tilt plus or minus up to 15 degrees) between horizontal and vertical planes; (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop; wherein the electromagnetic energy sensors are made with a silicone-based polymer (e.g. PDMS) which has been doped, impregnated, or coated with conductive material (e.g. carbon or silver).

In one embodiment, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in plane which is tilted substantially-midway (e.g. 45-degree-tilt plus or minus up to 15 degrees) between horizontal and vertical planes; (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop; wherein the electromagnetic energy sensors are made with PEDOT:PSS.

In an example, a wearable EEG device can comprise: (a) a soft (e.g. stretchable and elastic) headband which is worn around a person's head in a substantially-horizontal plane (e.g. horizontal plus or minus up to 15-degree tilt); (b) at least two relatively-flat electromagnetic energy sensors which are part of the headband; wherein the relatively-flat electromagnetic energy sensors are configured to be located on the person's forehead; wherein the relatively-flat electromagnetic energy sensors are made with PEDOT:PSS; and (c) at least two undulating and/or protruding electromagnetic energy sensors which are part of the headband; wherein the undulating and/or protruding electromagnetic energy sensors are configured to be located on the posterior half of the person's head; wherein the at least two undulating and/or protruding electromagnetic energy sensors are made with PEDOT:PSS; and wherein the sides of the at least two undulating and/or protruding electromagnetic energy sensors which face the person's head each further comprise a plurality of hair-penetrating undulations and/or protrusions.

In one embodiment, a wearable EEG device can comprise: (a) a semi-circular headband or hairband which is worn on a person's head in a substantially-vertical plane (e.g. vertical plus or minus up to 15-degree tilt); and (b) at least two undulating and/or protruding electromagnetic energy sensors which are part of the headband or hairband; wherein the undulating and/or protruding electromagnetic energy sensors are configured to be located on the upper half of the person's head; wherein the at least two undulating and/or protruding electromagnetic energy sensors are made with PEDOT:PSS; and wherein the sides of the at least two undulating and/or protruding electromagnetic energy sensors which face the person's head each further comprise a plurality of hair-penetrating undulations and/or protrusions. Alternatively, a wearable EEG device can comprise: (a) an upper loop which curves over the top of a person's head in a substantially-vertical plane (e.g. vertical plus or minus up to 15-degree tilt); and (b) electromagnetic energy sensors which are worn on the upper loop; wherein the sides of the electromagnetic energy sensors which face the person's head further comprise hair-penetrating protrusions.

In another example, a wearable EEG monitoring device can comprise: (a) a semi-circular headband or hairband which is worn on a person's head in a substantially-vertical plane (e.g. vertical plus or minus up to 15-degree tilt); and (b) two undulating and/or protruding electromagnetic energy sensors which are part of the headband or hairband; wherein the undulating and/or protruding electromagnetic energy sensors are configured to be located on the upper half of the person's head; and wherein the sides of the two undulating and/or protruding electromagnetic energy sensors which face the person's head each further comprise a plurality of hair-penetrating undulations and/or protrusions.

In an example, a wearable EEG device can comprise: (a) a soft (e.g. soft stretchable fabric) headband which is configured to be worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; and wherein the first electromagnetic energy sensor is made with a silicone-based polymer (e.g. PDMS) which has been doped, impregnated, or coated with conductive material (e.g. carbon or silver); and (c) a second electromagnetic energy sensor on the headband; wherein the second electromagnetic energy sensor is configured to be located on a second portion of the person's head; wherein the second portion is covered by hair; wherein the second electromagnetic energy sensor is made with a silicone-based polymer (e.g. PDMS) which has been doped, impregnated, or coated with conductive material (e.g. carbon or silver); and wherein a side of the second electromagnetic energy sensor which faces the person's head further comprises a plurality of hair-penetrating protrusions.

In another example, a wearable EEG device can comprise: (a) a soft (e.g. soft stretchable fabric) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; and wherein a side of the first electromagnetic energy sensor which faces the person's head has a first level of topological variation and/or undulation; and (c) a second electromagnetic energy sensor on the headband; wherein the second electromagnetic energy sensor is configured to be located on a second portion of the person's head; wherein a side of the second electromagnetic energy sensor which faces the person's head has a second level of topological variation and/or undulation; and wherein the second level of topological variation and/or undulation is greater than the first level of topological variation and/or undulation.

In an example, a wearable EEG device can comprise: (a) a soft (e.g. soft stretchable fabric) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on the person's forehead; and (c) a second electromagnetic energy sensor on the headband; wherein the second electromagnetic energy sensor is configured to be located on the posterior half of the person's head; and wherein a side of the second electromagnetic energy sensor which faces the person's head further comprises a plurality of hair-penetrating protrusions.

In one embodiment, a wearable EEG device can comprise: (a) a soft (e.g. stretchable and elastic) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on the person's forehead; wherein the first electromagnetic energy sensor is made with thermoplastic polyurethane which has been doped, impregnated, or coated with conductive material (e.g. carbon or silver); and wherein a side of the first electromagnetic energy sensor which faces the person's head has a first level of topological variation and/or undulation; and (c) a second electromagnetic energy sensor on the headband; wherein the second electromagnetic energy sensor is configured to be located on the posterior half of the person's head; wherein the second portion is covered by hair; wherein the second electromagnetic energy sensor is made with thermoplastic polyurethane which has been doped, impregnated, or coated with conductive material (e.g. carbon or silver); wherein a side of the second electromagnetic energy sensor which faces the person's head has a second level of topological variation and/or undulation; and wherein the second level of topological variation and/or undulation is greater than the first level of topological variation and/or undulation.

In an example, a wearable EEG monitoring device can comprise: (a) a soft (e.g. soft stretchable fabric) headband which is configured to be worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; and wherein the first electromagnetic energy sensor is made with a conductive hydrogel; and (c) a second electromagnetic energy sensor on the headband; wherein the second electromagnetic energy sensor is configured to be located on a second portion of the person's head; wherein the second portion is covered by hair; wherein the second electromagnetic energy sensor is made with a conductive hydrogel; and wherein a side of the second electromagnetic energy sensor which faces the person's head further comprises a plurality of hair-penetrating combs, teeth, prongs, or pins.

Alternatively, a wearable EEG device can comprise: (a) a soft (e.g. stretchable and elastic) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; and wherein the first electromagnetic energy sensor is made with PEDOT:PSS; and (c) a second electromagnetic energy sensor on the headband; wherein the second electromagnetic energy sensor is configured to be located on a second portion of the person's head; wherein the second portion is covered by hair; wherein the second electromagnetic energy sensor is made with PEDOT:PSS; and wherein a side of the second electromagnetic energy sensor which faces the person's head further comprises a plurality of hair-penetrating protrusions.

In an example, a wearable EEG device can comprise: (a) an upper loop which curves over the top of a person's head in a substantially-vertical plane (e.g. vertical plus or minus up to 15-degree tilt); (b) a rear loop which curves around the posterior of the person's head in a substantially-horizontal plane (e.g. horizontal plus or minus up to 15-degree tilt); and (c) a plurality of electromagnetic energy sensors on the upper loop and the rear loop; wherein the electromagnetic energy sensors are made with a silicone-based polymer (e.g. PDMS) which has been doped, impregnated, or coated with conductive material (e.g. carbon or silver). In another example, a wearable EEG device can comprise: (a) an upper loop which curves over the top of a person's head in a substantially-vertical plane (e.g. vertical plus or minus up to 15-degree tilt); (b) a rear loop which curves around the posterior of the person's head in a substantially-horizontal plane (e.g. horizontal plus or minus up to 15-degree tilt); and (c) a plurality of electromagnetic energy sensors on the upper loop and the rear loop; wherein the electromagnetic energy sensors are made with PEDOT:PSS.

In an example, a wearable EEG device can comprise: (a) an upper loop which curves over the top of a person's head in a substantially-vertical plane (e.g. vertical plus or minus up to 15-degree tilt); (b) a rear loop which curves around the posterior of the person's head in a substantially-horizontal plane (e.g. horizontal plus or minus up to 15-degree tilt); and (c) a plurality of undulating and/or protruding electromagnetic energy sensors on the upper loop and the rear loop; wherein the undulating and/or protruding electromagnetic energy sensors are made with a conductive hydrogel; and wherein the sides of the undulating and/or protruding electromagnetic energy sensors which face the person's head each further comprise a plurality of hair-penetrating undulations and/or protrusions.

In another example, a wearable EEG monitoring device can comprise: (a) a frontal loop which curves around a person's forehead in a substantially-horizontal plane (e.g. horizontal plus or minus up to 15-degree tilt); (b) an upper loop which curves over the top of a person's head in a substantially-vertical plane (e.g. vertical plus or minus up to 15-degree tilt); (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop; wherein the electromagnetic energy sensors are made with a silicone-based polymer (e.g. PDMS) which has been doped, impregnated, or coated with conductive material (e.g. carbon or silver).

In one embodiment, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in a substantially-vertical plane (e.g. vertical plus or minus up to 15-degree tilt); (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop; wherein the electromagnetic energy sensors are made with thermoplastic polyurethane which has been doped, impregnated, or coated with conductive material (e.g. carbon or silver).

In an example, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in plane which is tilted substantially-midway (e.g. 45-degree-tilt plus or minus up to 15 degrees) between horizontal and vertical planes; (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop.

In one embodiment, a wearable EEG device can comprise: (a) a soft (e.g. soft stretchable fabric) headband which is worn around a person's head in a substantially-horizontal plane (e.g. horizontal plus or minus up to 15-degree tilt); (b) at least two relatively-flat electromagnetic energy sensors which are part of the headband; wherein the relatively-flat electromagnetic energy sensors are configured to be located on the person's forehead; wherein the relatively-flat electromagnetic energy sensors are made with a silicone-based polymer (e.g. PDMS) which has been doped, impregnated, or coated with conductive material (e.g. carbon or silver); and (c) at least two undulating and/or protruding electromagnetic energy sensors which are part of the headband; wherein the undulating and/or protruding electromagnetic energy sensors are configured to be located on the posterior half of the person's head; wherein the at least two undulating and/or protruding electromagnetic energy sensors are made with a silicone-based polymer (e.g. PDMS) which has been doped, impregnated, or coated with conductive material (e.g. carbon or silver); and wherein the sides of the at least two undulating and/or protruding electromagnetic energy sensors which face the person's head each further comprise a plurality of hair-penetrating undulations and/or protrusions.

Alternatively, a wearable EEG monitoring device can comprise: (a) a semi-circular headband or hairband which is worn on a person's head in a substantially-vertical plane (e.g. vertical plus or minus up to 15-degree tilt); and (b) at least two undulating and/or protruding electromagnetic energy sensors which are part of the headband or hairband; wherein the undulating and/or protruding electromagnetic energy sensors are configured to be located on the upper half of the person's head; wherein the at least two undulating and/or protruding electromagnetic energy sensors are made with a silicone-based polymer (e.g. PDMS) which has been doped, impregnated, or coated with conductive material (e.g. carbon or silver); and wherein the sides of the at least two undulating and/or protruding electromagnetic energy sensors which face the person's head each further comprise a plurality of hair-penetrating undulations and/or protrusions.

In an example, a wearable EEG device can comprise: (a) a rear loop which curves around the posterior of a person's head in a substantially-horizontal plane (e.g. horizontal plus or minus up to 15-degree tilt); and (b) electromagnetic energy sensors which are worn on the rear loop; wherein the sides of the electromagnetic energy sensors which face the person's head further comprise hair-penetrating protrusions. In another example, a wearable EEG monitoring device can comprise: (a) an upper loop which curves over the top of a person's head in a substantially-vertical plane (e.g. vertical plus or minus up to 15-degree tilt); (b) electromagnetic energy sensors which are worn on the upper loop; wherein the sides of the electromagnetic energy sensors which face the person's head further comprise hair-penetrating protrusions; and (c) a rotating member which rotates the protrusions to help the protrusions move through the person's hair.

In an example, a wearable EEG device can comprise: (a) a soft (e.g. stretchable and elastic) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; and wherein the first electromagnetic energy sensor is made with a silicone-based polymer (e.g. PDMS) which has been doped, impregnated, or coated with conductive material (e.g. carbon or silver); and (c) a second electromagnetic energy sensor on the headband; wherein the second electromagnetic energy sensor is configured to be located on a second portion of the person's head; wherein the second portion is covered by hair; wherein the second electromagnetic energy sensor is made with a silicone-based polymer (e.g. PDMS) which has been doped, impregnated, or coated with conductive material (e.g. carbon or silver); and wherein a side of the second electromagnetic energy sensor which faces the person's head further comprises a plurality of hair-penetrating combs, teeth, prongs, or pins.

In another example, a wearable EEG device can comprise: (a) a soft (e.g. soft stretchable fabric) headband which is configured to be worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; and wherein the first portion is not covered by hair; and (c) a second electromagnetic energy sensor on the headband; wherein the second electromagnetic energy sensor is configured to be located on a second portion of the person's head; wherein the second portion is covered by hair; and wherein a side of the second electromagnetic energy sensor which faces the person's head further comprises a plurality of hair-penetrating protrusions.

In an example, a wearable EEG device can comprise: (a) a soft (e.g. soft stretchable fabric) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; wherein the first electromagnetic energy sensor is made with thermoplastic polyurethane which has been doped, impregnated, or coated with conductive material (e.g. carbon or silver); and wherein a side of the first electromagnetic energy sensor which faces the person's head has a first level of topological variation and/or undulation; and (c) a second electromagnetic energy sensor on the headband; wherein the second electromagnetic energy sensor is configured to be located on a second portion of the person's head; wherein the second portion is covered by hair; wherein the second electromagnetic energy sensor is made with thermoplastic polyurethane which has been doped, impregnated, or coated with conductive material (e.g. carbon or silver); wherein a side of the second electromagnetic energy sensor which faces the person's head has a second level of topological variation and/or undulation; and wherein the second level of topological variation and/or undulation is greater than the first level of topological variation and/or undulation.

In one embodiment, a wearable EEG device can comprise: (a) a soft (e.g. soft stretchable fabric) headband which is configured to be worn around a person's head in a substantially-horizontal plane (e.g. horizontal plus or minus up to 15-degree tilt); (b) a first electromagnetic energy sensor on the headband; wherein the first electromagnetic energy sensor is configured to be located on the person's forehead; and wherein the first electromagnetic energy sensor is made with thermoplastic polyurethane which has been doped, impregnated, or coated with conductive material (e.g. carbon or silver); and (c) a second electromagnetic energy sensor on the headband; wherein the second electromagnetic energy sensor is configured to be located on the posterior half of the person's head; wherein the second electromagnetic energy sensor is made with thermoplastic polyurethane which has been doped, impregnated, or coated with conductive material (e.g. carbon or silver); wherein a side of the second electromagnetic energy sensor which faces the person's head further comprises a plurality of hair-penetrating protrusions.

Alternatively, a wearable EEG monitoring device can comprise: (a) a soft (e.g. stretchable and elastic) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on the person's forehead; wherein the first electromagnetic energy sensor is made with a conductive hydrogel; and wherein a side of the first electromagnetic energy sensor which faces the person's head has a first level of topological variation and/or undulation; and (c) a second electromagnetic energy sensor on the headband; wherein the second electromagnetic energy sensor is configured to be located on the posterior half of the person's head; wherein the second portion is covered by hair; wherein the second electromagnetic energy sensor is made with a conductive hydrogel; wherein a side of the second electromagnetic energy sensor which faces the person's head has a second level of topological variation and/or undulation; and wherein the second level of topological variation and/or undulation is greater than the first level of topological variation and/or undulation.

In an example, a wearable EEG device can comprise: (a) a soft (e.g. soft stretchable fabric) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; and wherein the first electromagnetic energy sensor is made with PEDOT:PSS; and (c) a second electromagnetic energy sensor on the headband; wherein the second electromagnetic energy sensor is configured to be located on a second portion of the person's head; wherein the second portion is covered by hair; wherein the second electromagnetic energy sensor is made with PEDOT:PSS; and wherein a side of the second electromagnetic energy sensor which faces the person's head further comprises a plurality of hair-penetrating combs, teeth, prongs, or pins. In an example, a wearable EEG device can comprise: (a) an upper loop which curves over the top of a person's head in a substantially-vertical plane (e.g. vertical plus or minus up to 15-degree tilt); (b) a rear loop which curves around the posterior of the person's head in a substantially-horizontal plane (e.g. horizontal plus or minus up to 15-degree tilt); and (c) a plurality of electromagnetic energy sensors on the upper loop and the rear loop.

In another example, a wearable EEG device can comprise: (a) an upper loop which curves over the top of a person's head in a substantially-vertical plane (e.g. vertical plus or minus up to 15-degree tilt); (b) a rear loop which curves around the posterior of the person's head in a substantially-horizontal plane (e.g. horizontal plus or minus up to 15-degree tilt); and (c) a plurality of undulating and/or protruding electromagnetic energy sensors on the upper loop and the rear loop; wherein the undulating and/or protruding electromagnetic energy sensors are made with a silicone-based polymer (e.g. PDMS) which has been doped, impregnated, or coated with conductive material (e.g. carbon or silver); and wherein the sides of the undulating and/or protruding electromagnetic energy sensors which face the person's head each further comprise a plurality of hair-penetrating undulations and/or protrusions.

In one embodiment, a wearable EEG monitoring device can comprise: (a) an upper loop which curves over the top of a person's head in a substantially-vertical plane (e.g. vertical plus or minus up to 15-degree tilt); (b) a rear loop which curves around the posterior of the person's head in a substantially-horizontal plane (e.g. horizontal plus or minus up to 15-degree tilt); and (c) a plurality of undulating and/or protruding electromagnetic energy sensors on the upper loop and the rear loop; wherein the undulating and/or protruding electromagnetic energy sensors are made with PEDOT:PSS; and wherein the sides of the undulating and/or protruding electromagnetic energy sensors which face the person's head each further comprise a plurality of hair-penetrating undulations and/or protrusions.

In another example, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in a substantially-vertical plane (e.g. vertical plus or minus up to 15-degree tilt); (c) a rear loop which curves around the posterior of the person's head in a substantially-horizontal plane (e.g. horizontal plus or minus up to 15-degree tilt); (d) a first set of electromagnetic energy sensors which are worn on the frontal loop; wherein the sides of the first set of electromagnetic energy sensors which face the person's head have a first average level of topological variation or undulation; and (e) a second set of electromagnetic energy sensors which are worn on the upper loop and the rear loop; wherein the sides of the second set of electromagnetic energy sensors which face the person's head have a second average level of topological variation or undulation; and wherein the second level is greater than the first level.

In an example, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in a substantially-vertical plane (e.g. vertical plus or minus up to 15-degree tilt); (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop; wherein the electromagnetic energy sensors are made with a conductive hydrogel.

In another example, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in plane which is tilted substantially-midway (e.g. 45-degree-tilt plus or minus up to 15 degrees) between horizontal and vertical planes; (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop; wherein the electromagnetic energy sensors are made with thermoplastic polyurethane which has been doped, impregnated, or coated with conductive material (e.g. carbon or silver).

Alternatively, a wearable EEG monitoring device can comprise: (a) a soft (e.g. stretchable and elastic) headband which is configured to be worn around a person's head in a substantially-horizontal plane (e.g. horizontal plus or minus up to 15-degree tilt); (b) at least two relatively-flat electromagnetic energy sensors which are part of the headband; wherein the relatively-flat electromagnetic energy sensors are configured to be located on the person's forehead; wherein the relatively-flat electromagnetic energy sensors are made with thermoplastic polyurethane which has been doped, impregnated, or coated with conductive material (e.g. carbon or silver); and (c) at least two undulating and/or protruding electromagnetic energy sensors which are part of the headband; wherein the undulating and/or protruding electromagnetic energy sensors are configured to be located on the posterior half of the person's head; wherein the at least two undulating and/or protruding electromagnetic energy sensors are made with thermoplastic polyurethane which has been doped, impregnated, or coated with conductive material (e.g. carbon or silver); and wherein the sides of the at least two undulating and/or protruding electromagnetic energy sensors which face the person's head each further comprise a plurality of hair-penetrating undulations and/or protrusions.

In an example, a wearable EEG device can comprise: (a) a semi-circular headband or hairband which is worn on a person's head in a substantially-vertical plane (e.g. vertical plus or minus up to 15-degree tilt); and (b) at least two undulating and/or protruding electromagnetic energy sensors which are part of the headband or hairband; wherein the undulating and/or protruding electromagnetic energy sensors are configured to be located on the upper half of the person's head; wherein the at least two undulating and/or protruding electromagnetic energy sensors are made with thermoplastic polyurethane which has been doped, impregnated, or coated with conductive material (e.g. carbon or silver); and wherein the sides of the at least two undulating and/or protruding electromagnetic energy sensors which face the person's head each further comprise a plurality of hair-penetrating undulations and/or protrusions.

In one embodiment, a wearable EEG device can comprise: (a) a rear loop which curves around the posterior of a person's head in a substantially-horizontal plane (e.g. horizontal plus or minus up to 15-degree tilt); (b) electromagnetic energy sensors which are worn on the rear loop; wherein the sides of the electromagnetic energy sensors which face the person's head further comprise hair-penetrating protrusions; and (c) a rotating member which rotates the protrusions to help the protrusions move through the person's hair. In an example, a wearable EEG device can comprise: (a) an upper loop which curves over the top of a person's head in a substantially-vertical plane (e.g. vertical plus or minus up to 15-degree tilt); (b) electromagnetic energy sensors which are worn on the upper loop; wherein the sides of the electromagnetic energy sensors which face the person's head further comprise hair-penetrating protrusions; and (c) a vibrating member which vibrates the protrusions to help the protrusions move through the person's hair.

In an example, a wearable EEG monitoring device can comprise: (a) a soft (e.g. soft stretchable fabric) headband which is configured to be worn around a person's head in a substantially-horizontal plane (e.g. horizontal plus or minus up to 15-degree tilt); (b) a first electromagnetic energy sensor on the headband; wherein the first electromagnetic energy sensor is configured to be located on the person's forehead; wherein the first electromagnetic energy sensor is made with a silicone-based polymer (e.g. PDMS) which has been doped, impregnated, or coated with conductive material (e.g. carbon or silver); and wherein a side of the first electromagnetic energy sensor which faces the person's head has a first level of topological variation and/or undulation; and (c) a second electromagnetic energy sensor on the headband; wherein the second electromagnetic energy sensor is configured to be located on the posterior half of the person's head; wherein the second portion is covered by hair; wherein the second electromagnetic energy sensor is made with a silicone-based polymer (e.g. PDMS) which has been doped, impregnated, or coated with conductive material (e.g. carbon or silver); wherein a side of the second electromagnetic energy sensor which faces the person's head has a second level of topological variation and/or undulation; and wherein the second level of topological variation and/or undulation is greater than the first level of topological variation and/or undulation.

In another example, a wearable EEG device can comprise: (a) a soft (e.g. stretchable and elastic) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; and (c) a second electromagnetic energy sensor on the headband; wherein the second electromagnetic energy sensor is configured to be located on a second portion of the person's head; and wherein a side of the second electromagnetic energy sensor which faces the person's head further comprises a plurality of hair-penetrating prongs, teeth, or combs.

In one embodiment, a wearable EEG device can comprise: (a) a soft (e.g. soft stretchable fabric) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; and wherein the first electromagnetic energy sensor is made with thermoplastic polyurethane which has been doped, impregnated, or coated with conductive material (e.g. carbon or silver); and (c) a second electromagnetic energy sensor on the headband; wherein the second electromagnetic energy sensor is configured to be located on a second portion of the person's head; wherein the second portion is covered by hair; wherein the second electromagnetic energy sensor is made with thermoplastic polyurethane which has been doped, impregnated, or coated with conductive material (e.g. carbon or silver); and wherein a side of the second electromagnetic energy sensor which faces the person's head further comprises a plurality of hair-penetrating protrusions.

In another example, a wearable EEG device can comprise: (a) a soft (e.g. soft stretchable fabric) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on a first portion of the person's head; wherein the first portion is not covered by hair; wherein the first electromagnetic energy sensor is made with a conductive hydrogel; and wherein a side of the first electromagnetic energy sensor which faces the person's head has a first level of topological variation and/or undulation; and (c) a second electromagnetic energy sensor on the headband; wherein the second electromagnetic energy sensor is configured to be located on a second portion of the person's head; wherein the second portion is covered by hair; wherein the second electromagnetic energy sensor is made with a conductive hydrogel; wherein a side of the second electromagnetic energy sensor which faces the person's head has a second level of topological variation and/or undulation; and wherein the second level of topological variation and/or undulation is greater than the first level of topological variation and/or undulation.

Alternatively, a wearable EEG monitoring device can comprise: (a) a soft (e.g. soft stretchable fabric) headband which is configured to be worn around a person's head in a substantially-horizontal plane (e.g. horizontal plus or minus up to 15-degree tilt); (b) a first electromagnetic energy sensor on the headband; wherein the first electromagnetic energy sensor is configured to be located on the person's forehead; and wherein the first electromagnetic energy sensor is made with a conductive hydrogel; and (c) a second electromagnetic energy sensor on the headband; wherein the second electromagnetic energy sensor is configured to be located on the posterior half of the person's head; wherein the second electromagnetic energy sensor is made with a conductive hydrogel; wherein a side of the second electromagnetic energy sensor which faces the person's head further comprises a plurality of hair-penetrating protrusions.

In one embodiment, a wearable EEG device can comprise: (a) a soft (e.g. soft stretchable fabric) headband which is worn around a person's head in a substantially-horizontal plane (e.g. horizontal plus or minus up to 15-degree tilt); (b) a first electromagnetic energy sensor on the headband; wherein the first electromagnetic energy sensor is configured to be located on the person's forehead; wherein the first electromagnetic energy sensor is made with PEDOT:PSS; and wherein a side of the first electromagnetic energy sensor which faces the person's head has a first level of topological variation and/or undulation; and (c) a second electromagnetic energy sensor on the headband; wherein the second electromagnetic energy sensor is configured to be located on the posterior half of the person's head; wherein the second portion is covered by hair; wherein the second electromagnetic energy sensor is made with PEDOT:PSS; wherein a side of the second electromagnetic energy sensor which faces the person's head has a second level of topological variation and/or undulation; and wherein the second level of topological variation and/or undulation is greater than the first level of topological variation and/or undulation.

In an example, a wearable EEG monitoring device can comprise: (a) an upper loop which curves over the top of a person's head in a substantially-vertical plane (e.g. vertical plus or minus up to 15-degree tilt); (b) a rear loop which curves around the posterior of the person's head in a substantially-horizontal plane (e.g. horizontal plus or minus up to 15-degree tilt); and (c) a plurality of electromagnetic energy sensors on the upper loop and the rear loop; wherein the electromagnetic energy sensors are made with thermoplastic polyurethane which has been doped, impregnated, or coated with conductive material (e.g. carbon or silver). Alternatively, a wearable EEG device can comprise: (a) an upper loop which curves over the top of a person's head in a substantially-vertical plane (e.g. vertical plus or minus up to 15-degree tilt); (b) a rear loop which curves around the posterior of the person's head in a substantially-horizontal plane (e.g. horizontal plus or minus up to 15-degree tilt); and (c) a plurality of undulating and/or protruding electromagnetic energy sensors on the upper loop and the rear loop; wherein the sides of the undulating and/or protruding electromagnetic energy sensors which face the person's head each further comprise a plurality of hair-penetrating undulations and/or protrusions.

In another example, a wearable EEG device can comprise: (a) a frontal loop which curves around a person's forehead in a plane which is tilted substantially-midway (e.g. 45-degree-tilt plus or minus up to 15 degrees) between horizontal and vertical planes; (b) an upper loop which curves over the top of a person's head in a substantially-vertical plane (e.g. vertical plus or minus up to 15-degree tilt); (c) a rear loop which curves around the posterior of the person's head in a substantially-horizontal plane (e.g. horizontal plus or minus up to 15-degree tilt); (d) a first set of electromagnetic energy sensors which are worn on the frontal loop; wherein the sides of the first set of electromagnetic energy sensors which face the person's head have a first average level of topological variation or undulation; and (e) a second set of electromagnetic energy sensors which are worn on the upper loop and the rear loop; wherein the sides of the second set of electromagnetic energy sensors which face the person's head have a second average level of topological variation or undulation; and wherein the second level is greater than the first level.

In an example, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in a substantially-vertical plane (e.g. vertical plus or minus up to 15-degree tilt); (c) a rear loop which curves around the posterior of the person's head in a substantially-horizontal plane (e.g. horizontal plus or minus up to 15-degree tilt); (d) a first set of electromagnetic energy sensors which are worn on the frontal loop; wherein the sides of the first set of electromagnetic energy sensors which face the person's head are relatively flat; and (e) a second set of electromagnetic energy sensors which are worn on the upper loop and the rear loop; wherein the sides of the second set of electromagnetic energy sensors which face the person's head further comprise hair-penetrating protrusions.

In another example, a wearable EEG monitoring device can comprise: (a) a frontal loop which curves around a person's forehead in a substantially-horizontal plane (e.g. horizontal plus or minus up to 15-degree tilt); (b) an upper loop which curves over the top of a person's head in a substantially-vertical plane (e.g. vertical plus or minus up to 15-degree tilt); (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop; wherein the electromagnetic energy sensors are made with PEDOT:PSS.

In one embodiment, a wearable EEG device can comprise: (a) a frontal loop which curves around a person's forehead in a substantially-horizontal plane (e.g. horizontal plus or minus up to 15-degree tilt); (b) an upper loop which curves over the top of a person's head in plane which is tilted substantially-midway (e.g. 45-degree-tilt plus or minus up to 15 degrees) between horizontal and vertical planes; (c) a rear loop which curves around the posterior of the person's head in a substantially-horizontal plane (e.g. horizontal plus or minus up to 15-degree tilt); and (d) a plurality of electromagnetic energy sensors on the frontal loop, the upper loop, and the rear loop; wherein the electromagnetic energy sensors are made with a conductive hydrogel.

In an example, a wearable EEG monitoring device can comprise: (a) a soft (e.g. soft stretchable fabric) headband which is worn around a person's head in a substantially-horizontal plane (e.g. horizontal plus or minus up to 15-degree tilt); (b) at least two relatively-flat electromagnetic energy sensors which are part of the headband; wherein the relatively-flat electromagnetic energy sensors are configured to be located on the person's forehead; wherein the relatively-flat electromagnetic energy sensors are made with a conductive hydrogel; and (c) at least two undulating and/or protruding electromagnetic energy sensors which are part of the headband; wherein the undulating and/or protruding electromagnetic energy sensors are configured to be located on the posterior half of the person's head; wherein the at least two undulating and/or protruding electromagnetic energy sensors are made with a conductive hydrogel; and wherein the sides of the at least two undulating and/or protruding electromagnetic energy sensors which face the person's head each further comprise a plurality of hair-penetrating undulations and/or protrusions.

In an example, a wearable EEG device can comprise: (a) a semi-circular headband or hairband which is worn on a person's head in a substantially-vertical plane (e.g. vertical plus or minus up to 15-degree tilt); and (b) at least two undulating and/or protruding electromagnetic energy sensors which are part of the headband or hairband; wherein the undulating and/or protruding electromagnetic energy sensors are configured to be located on the upper half of the person's head; wherein the at least two undulating and/or protruding electromagnetic energy sensors are made with a conductive hydrogel; and wherein the sides of the at least two undulating and/or protruding electromagnetic energy sensors which face the person's head each further comprise a plurality of hair-penetrating undulations and/or protrusions.

Alternatively, a wearable EEG device can comprise: (a) a rear loop which curves around the posterior of a person's head in a substantially-horizontal plane (e.g. horizontal plus or minus up to 15-degree tilt); (b) electromagnetic energy sensors which are worn on the rear loop; wherein the sides of the electromagnetic energy sensors which face the person's head further comprise hair-penetrating protrusions; and (c) a vibrating member which vibrates the protrusions to help the protrusions move through the person's hair.

In an example, an electromagnetic energy sensor can comprise 2 pairs of conductive polymer electrodes. In an example, an electromagnetic energy sensor can comprise 4 pairs of conductive polymer electrodes. In an example, an electromagnetic energy sensor can comprise 6 pairs of conductive polymer electrodes. In an example, an electromagnetic energy sensor can comprise 8 pairs of conductive polymer electrodes.

In another example, a wearable EEG device can comprise: (a) a soft (e.g. stretchable and elastic) headband which is worn around a person's head in a substantially-horizontal plane (e.g. horizontal plus or minus up to 15-degree tilt); (b) two relatively-flat electromagnetic energy sensors which are part of the headband; wherein the relatively-flat electromagnetic energy sensors are configured to be located on the person's forehead; and (c) two undulating and/or protruding electromagnetic energy sensors which are part of the headband; wherein the undulating and/or protruding electromagnetic energy sensors are configured to be located on the posterior half of the person's head; and wherein the sides of the two undulating and/or protruding electromagnetic energy sensors which face the person's head each further comprise a plurality of hair-penetrating undulations and/or protrusions.

In an example, an electromagnetic energy sensor can comprise a conductive layer and a non-conductive layer. In an example, an electromagnetic energy sensor can comprise a first layer with a first conductivity layer and a second layer with a second conductivity layer, wherein the second conductivity level is less than the first conductivity level. In an example, an electromagnetic energy sensor can comprise a flexible electroconductive layer and a flexible non-electroconductive layer. In an example, these two layers can be substantially parallel. In an example, an electromagnetic energy sensor can include a high-conductivity layer which is worn closer to the surface of a person's head and a low-conductivity layer which is worn farther from the surface of the person's head. In an example, an electromagnetic energy sensor can include two flexible conductive layers separated by a flexible non-conductive layer. In an example, these three layers can be substantially parallel.

In an example, an electromagnetic energy sensor can comprise alternating layers of low-conductivity material and high-conductivity material. In an example, an electromagnetic energy sensor can comprise two conductive layers separated by an insulating layer. In an example, an electromagnetic energy sensor can include two flexible layers of high-conductivity material separated by a flexible layer of low-conductivity material. In an example, an electromagnetic energy sensor can include two flexible layers of high-conductivity material separated by a space or gap. In an example, an electromagnetic energy sensor can include a dielectric layer. In an example, an electromagnetic energy sensor can comprise an adhesive layer which is adhered to a conductive layer. In an example, an electromagnetic energy sensor can include a capacitive layer. In an example, an electromagnetic energy sensor can include a capacitive electrode. In an example, an electromagnetic energy sensor can comprise an inner low-conductivity layer and an outer high-conductivity layer.

In an example, an electromagnetic energy sensor can comprise an inner high-conductivity layer and an outer low-conductivity layer. In an example, an electromagnetic energy sensor can include one or more hair-penetrating protrusions with inner low-conductivity cores and outer high-conductivity coatings or layers. In an example, an electromagnetic energy sensor can include one or more hair-penetrating protrusions with inner high-conductivity cores and outer low-conductivity coatings or layers.

In an example, an electromagnetic energy sensor can comprise high-conductivity and low-conductivity layers with different thicknesses. In an example, a non-conductive layer can be thinner than a conductive layer. Alternatively, a non-conductive layer can be thicker than a conductive layer. In an example, an electromagnetic energy sensor can comprise high-conductivity and low-conductivity layers with different orientations. In an example, conductive strands with selected orientations can be embedded in non-conductive material. In an example, longitudinal conductive strands with a plurality of orientations can be embedded in non-conductive material. In an example, conductive rings can be embedded in non-conductive material.

In an example, an electromagnetic energy sensor can comprise two conductive layers separated by a dielectric elastomer. In an example, an electromagnetic energy sensor can comprise conductive layers with a dielectric coating. In an example, an electromagnetic energy sensor can comprise a dielectric coating between conductive layers. In an example, an electromagnetic energy sensor can comprise a dielectric layer between conductive layers. In an example, an electromagnetic energy sensor can comprise a flexible dielectric coating between conductive layers. In an example, an electromagnetic energy sensor can comprise a flexible dielectric layer between conductive layers. In an example, an electromagnetic energy sensor can comprise a flexible dielectric elastomer between conductive layers. In an example, an electromagnetic energy sensor can comprise high-conductivity and low-conductivity layers with different elasticity levels.

In an example, an electromagnetic energy sensor can include a low-conductivity layer which is worn closer to the surface of a person's head and a high-conductivity layer which is worn farther from the surface of the person's head. In an example, an electromagnetic energy sensor can include a low-conductivity layer which is configured to be worn a first average distance from the surface of a person's head and a high-conductivity layer which is configured to be worn a second average distance from the surface of the person's head, wherein the second average distance is greater than the first average distance.

In an example, an electromagnetic energy sensor which is worn on a portion of a person's head which is covered with hair can comprise a base which is configured to have an orientation which is generally parallel to the surface of a person's head and 2 to 4 hair-penetrating and/or hair-engaging protrusions which extend inward from the base toward the surface of the person's head. In an example, the protrusions can extend between strands (and/or under layers) of the person's hair and achieve better electromagnetic communication with the person's brain. In an example, an electromagnetic energy sensor which is worn on a portion of a person's head which is covered with hair can comprise a base with an orientation which is generally parallel to the surface of a person's head and 2 to 4 hair-penetrating and/or hair-engaging protrusions with longitudinal axes which are generally orthogonal to the surface of the person's head. In an example, an electromagnetic energy sensor can comprise 2 pairs of conductive hair-penetrating protrusions.

In an example, an electromagnetic energy sensor which is worn on a portion of a person's head which is covered with hair can comprise a base which is configured to have an orientation which is generally parallel to the surface of a person's head and 5 to 8 hair-penetrating and/or hair-engaging protrusions which extend inward from the base toward the surface of the person's head. In an example, the protrusions can extend between strands (and/or under layers) of the person's hair and achieve better electromagnetic communication with the person's brain. In an example, an electromagnetic energy sensor which is worn on a portion of a person's head which is covered with hair can comprise a base with an orientation which is generally parallel to the surface of a person's head and 5 to 8 hair-penetrating and/or hair-engaging protrusions with longitudinal axes which are generally orthogonal to the surface of the person's head. In an example, an electromagnetic energy sensor can comprise 4 pairs of conductive hair-penetrating protrusions.

In an example, an electromagnetic energy sensor can comprise a base which is a first distance from a person's head and a plurality of conductive protrusions, prongs, teeth, combs, pins, and/or petals which extend inward to a second distance from the person's head, wherein the second distance is between 50% and 90% of the first distance. In an example, an electromagnetic energy sensor can comprise a base which is a first distance from a person's head and a plurality of conductive protrusions, prongs, teeth, combs, pins, and/or petals which extend inward to a second distance from the person's head, wherein the second distance is between 10% and 50% of the first distance.

In an example, an electromagnetic energy sensor can comprise a plurality of hair-penetrating protrusions which are in electromagnetic communication with a person's brain. In an example, an electromagnetic energy sensor can comprise a base which is worn a first distance from the surface of a person's head and a plurality of protrusions which extend inward from the base through the person's hair to a second distance from the surface of the person's head, wherein the second distance is less than the first distance. In an example, an electromagnetic energy sensor can comprise a base which is worn a first distance from the surface of a person's head and two protrusions which extend inward from the base through the person's hair to a second distance from the surface of the person's head, wherein the second distance is less than the first distance.

In an example, an electromagnetic energy sensor can comprise one or more protrusions, prongs, teeth, combs, pins, and/or petals with longitudinal axes which have generally-vertical orientations when they are worn on a person's head. In an example, an electromagnetic energy sensor can comprise one or more protrusions, prongs, teeth, combs, pins, and/or petals with longitudinal cross-sectional perimeters which have generally-vertical orientations when worn on a person's head. In an example, a conductive hair-penetrating protrusion can have a flexible conductive core which is covered with non-conductive material. In an example, a flexible conductive core of a hair-penetrating protrusion can be made from PDMS, TPU, or PEDOT:PSS. In an example, a flexible conductive core of a hair-penetrating protrusion can be made from a hydrogel. In an example, a wearable EEG device can have at least 10 hair-penetrating conductive protrusions.

In an example, a wearable mobile device for monitoring electromagnetic brain activity can include an array of conductive hair-penetrating conductive protrusions. In an example, these protrusions can protrude inward from a base toward the surface of a person's head at an acute angle relative to the base, instead of a conventional perpendicular orientation. In an example, these protrusions can protrude inward from a base toward the surface of a person's head at different angles. In an example, protrusions in such an array can protrude toward a person's head can be tilted toward the periphery of the array. In an example, protrusions can be tilted in a partially-radial manner, away from the center of the array and toward the perimeter of the array. In this manner, the protrusions can better penetrate through layers of hair without creating uncomfortable points of high-pressure contact with the person's head. In an example, protrusions can be angled toward the periphery of the array in a windmill, whorl, or whorl-windmill pattern.

In an example, an electromagnetic energy sensor can comprise one or more protrusions, prongs, teeth, combs, pins, and/or petals which are upwardly inserted into a person's hair. In an example, an electromagnetic energy sensor can comprise one or more protrusions, prongs, teeth, combs, pins, and/or petals which are inserted into a person's hair with an upward motion. In an example, an electromagnetic energy sensor can comprise one or more protrusions, prongs, teeth, combs, pins, and/or petals which face upward when worn on a person's head. In an example, an electromagnetic energy sensor can comprise one or more protrusions, prongs, teeth, combs, pins, and/or petals which face inward and upward when worn on a person's head. In an example, a wearable EEG device can have a plurality of hair-penetrating protrusions which are inserted under the outer layer of a person's hair so that they are generally not visible. In an example, an electromagnetic energy sensor can comprise a base which is worn outside a layer of a person's hair and one or more protrusions, prongs, teeth, combs, pins, and/or petals which are worn interior to (or within) the layer so that they are not visible.

In an example, a mobile wearable EEG device can comprise at least 10 hair-penetrating or hair-engaging conductive protrusions which extend into a person's hair in order to be in good electromagnetic communication with the person's brain. In an example, an electromagnetic energy sensor can comprise 6 pairs of conductive hair-penetrating protrusions. In an example, an electromagnetic energy sensor can comprise 8 pairs of conductive hair-penetrating protrusions. In an example, protrusions, prongs, teeth, combs, pins, and/or petals can face inward toward a person's head from a base which is worn on the person's head. In an example, an electromagnetic energy sensor can comprise one or more protrusions, prongs, teeth, combs, pins, and/or petals with longitudinal axes which have generally-horizontal orientations when they are worn on a person's head.

In an example, an electromagnetic energy sensor can comprise a base which is generally parallel to the surface of a person's head and one or more protrusions, prongs, teeth, combs, pins, and/or petals which are generally orthogonal to the surface of the person's head. In an example, an electromagnetic energy sensor can include: a base; a first hair-penetrating protrusion which extends at a first angle from the base toward the surface of a person's head; and a second hair-penetrating protrusion which extends at a second angle from the base toward the surface of a person's head, wherein the second angle differs from the first angle by at least 10 degrees. In an example, an electromagnetic energy sensor can include: a base; a first hair-penetrating protrusion which extends at a first angle from the base toward the surface of a person's head; and a second hair-penetrating protrusion which extends at a second angle from the base toward the surface of a person's head, wherein the second angle differs from the first angle by 10 to 45 degrees. In an example, each sensor can have 4 to 6 hair-penetrating or hair-engaging conductive protrusions. In an example, an electromagnetic energy sensor can comprise 3 pairs of conductive hair-penetrating protrusions.

In an example, an electromagnetic energy sensor can comprise: a base which is worn outside a layer of a person's hair; and one or more conductive protrusions which extend inward from the base through the layer to be in electromagnetic communication with the person's brain. In an example, an electromagnetic energy sensor can comprise: a base which is worn outside a layer of a person's hair; and one or more conductive strips which extend inward from the base through the layer to be in electromagnetic communication with the person's brain.

In an example, an electromagnetic energy sensor can comprise: a base which is worn outside a layer of a person's hair; and one or more conductive protrusions which extend inward from the base through the layer at different angles. In an example, an electromagnetic energy sensor can comprise: a base which is worn outside a layer of a person's hair; and one or more pairs of conductive protrusions which extend inward from the base through the layer at different angles. In an example, an electromagnetic energy sensor can comprise: a base which is worn outside a layer of a person's hair; and one or more pairs of tapered conductive protrusions which extend inward from the base through the layer at different angles.

In an example, an electromagnetic energy sensor can comprise: a base which is worn outside a layer of a person's hair; and one or more pairs of generally-parallel conductive protrusions which extend inward from the base through the layer at different angles. In an example, an electromagnetic energy sensor can comprise: a base which is worn outside a layer of a person's hair; and one or more pairs of generally-parallel linear conductive protrusions which extend inward from the base through the layer at different angles. In an example, an electromagnetic energy sensor can comprise: a base which is worn outside a layer of a person's hair; and one or more pairs of conductive protrusions with hydrogel tips which extend inward from the base through the layer at different angles.

In an example, an electromagnetic energy sensor can comprise a base which is worn a first distance from the surface of a person's head and at least two protrusions which extend inward from the base through the person's hair to a second distance from the surface of the person's head, wherein the second distance is less than the first distance. In an example, an electromagnetic energy sensor can comprise a base which is worn a first distance from the surface of a person's head and 2 to 4 protrusions which extend inward from the base through the person's hair to a second distance from the surface of the person's head, wherein the second distance is less than the first distance.

In an example, an electromagnetic energy sensor can comprise a base which is worn a first distance from the surface of a person's head and 4 to 8 protrusions which extend inward from the base through the person's hair to a second distance from the surface of the person's head, wherein the second distance is less than the first distance. In an example, an electromagnetic energy sensor can comprise a base which is worn a first distance from the surface of a person's head and 2 to 4 pairs of conductive protrusions which extend inward from the base through the person's hair to a second distance from the surface of the person's head, wherein the second distance is less than the first distance.

In an example, an electromagnetic energy sensor can comprise a base which is worn a first distance from the surface of a person's head and 4 to 8 pairs of conductive protrusions which extend inward from the base through the person's hair to a second distance from the surface of the person's head, wherein the second distance is less than the first distance.

In an example, an electromagnetic energy sensor can comprise a base which is worn a first distance from the surface of a person's head and a plurality of conductive protrusions which extend inward from the base through the person's hair to a second distance from the surface of the person's head, wherein the second distance is less than the first distance, and wherein the conductive protrusions are 10 mm to 30 mm apart from each other. In an example, an electromagnetic energy sensor can comprise a base which is worn a first distance from the surface of a person's head and a plurality of conductive protrusions which extend inward from the base through the person's hair to a second distance from the surface of the person's head, wherein the second distance is less than the first distance, and wherein the conductive protrusions are 5 mm to 20 mm apart from each other.

In an example, an electromagnetic energy sensor can comprise a base which is worn a first distance from the surface of a person's head and a plurality of conductive protrusions which extend inward from the base through the person's hair to a second distance from the surface of the person's head, wherein the second distance is less than the first distance, and wherein the conductive protrusions are 20 mm to 100 mm apart from each other. In an example, an electromagnetic energy sensor can comprise a base which is worn a first distance from the surface of a person's head and a plurality of conductive protrusions which extend inward from the base through the person's hair to a second distance from the surface of the person's head, wherein the second distance is 5 mm to 20 mm less than the first distance.

In an example, an electromagnetic energy sensor can comprise a base which is worn a first distance from the surface of a person's head and a plurality of conductive protrusions which extend inward from the base through the person's hair to a second distance from the surface of the person's head, wherein the second distance is 20 mm to 50 mm less than the first distance. In an example, an electromagnetic energy sensor can comprise a base which is worn a first distance from the surface of a person's head and a plurality of conductive protrusions which extend inward from the base through the person's hair to a second distance from the surface of the person's head, wherein the second distance is 1 mm to 10 mm less than the first distance.

In an example, an electromagnetic energy sensor can comprise a base which is worn a first distance from the surface of a person's head and a plurality of tapered conductive protrusions which extend inward from the base through the person's hair to a second distance from the surface of the person's head, wherein the second distance is less than the first distance, and wherein portions of the tapered conductive protrusions which are closest to the surface of the person's head have a smaller cross-sectional perimeter than portions of the tapered conductive protrusions which are farthest from the surface of the person's head.

In an example, an electromagnetic energy sensor can include a first hair-penetrating protrusion made from a first material and a second hair-penetrating protrusion made from a second material. In an example, an electromagnetic energy sensor can include a first hair-penetrating protrusion with a first conductivity level and a second hair-penetrating protrusion with a second conductivity level, wherein the second level is greater than the first level. In an example, an electromagnetic energy sensor can include a first hair-penetrating protrusion with a first resistance level and a second hair-penetrating protrusion with a second resistance level, wherein the second level is greater than the first level.

In an example, an electromagnetic energy sensor can include a first hair-penetrating protrusion with a first durometer level and a second hair-penetrating protrusion with a second durometer level, wherein the second level is greater than the first level. In an example, an electromagnetic energy sensor can include a first hair-penetrating protrusion made from a first mixture of a non-conductive polymer and a conductive material and a second hair-penetrating protrusion made from a second mixture of a non-conductive polymer and a conductive material, wherein the second mixture is different than the first material. In an example, an electromagnetic energy sensor can include a first hair-penetrating protrusion made with a first proportion of a non-conductive polymer and conductive material and a second hair-penetrating protrusion made with a second proportion of the non-conductive polymer and the conductive material, wherein the second proportion is different than the first proportion.

In an example, an electromagnetic energy sensor can include a first hair-penetrating protrusion with a first shape and a second hair-penetrating protrusion with a second shape. In an example, an electromagnetic energy sensor can include a first hair-penetrating protrusion with a first cross-sectional shape and a second hair-penetrating protrusion with a second cross-sectional shape, wherein the second cross-sectional shape is different than the first cross-sectional shape. In an example, an electromagnetic energy sensor can include a first hair-penetrating protrusion with a first shape and a second hair-penetrating protrusion with a second shape, wherein the second protrusion is more arcuate than the first protrusion.

In an example, an electromagnetic energy sensor can include a first hair-penetrating protrusion with a first shape and a second hair-penetrating protrusion with a second shape, wherein the second protrusion is larger than the first protrusion. In an example, an electromagnetic energy sensor can include a first hair-penetrating protrusion with a first shape and a second hair-penetrating protrusion with a second shape, wherein the second protrusion is longer than the first protrusion. In an example, an electromagnetic energy sensor can include a first hair-penetrating protrusion with a first cross-sectional size and a second hair-penetrating protrusion with a second cross-sectional size, wherein the second size is greater than the first size.

In an example, a mobile wearable device with electromagnetic energy sensors can include a plurality of conductive protrusions which extend inward from a base toward the surface of a person's head. In an example, these conductive protrusions can be combs, teeth, prongs, protuberances, or undulations. In example, these conductive protrusions are sufficiently resilient and rigid to penetrate through layers of a person's hair, but not so resilient and rigid that they cause discomfort as they contact the person's head. In an example, these conductive protrusions are sufficiently soft and deformable that they do not cause discomfort as they contact the surface of a person's head, but not so soft and deformable that they do not penetrate through layers of the person's hair. In example, these conductive protrusions are sufficiently resilient and rigid to penetrate through layers of a person's hair, but also sufficiently soft and deformable that they do not cause discomfort as they contact the person's head.

In an example, a plurality of conductive hair-penetrating protrusions can be configured in a single ring. In an example, a plurality of conductive hair-penetrating protrusions can be configured in nested rings. In an example, a plurality of conductive hair-penetrating protrusions can be configured in nested (e.g. concentric) rings, wherein each protrusion is ring-shaped and wherein the ring-shaped protrusions are nested (e.g. concentric) within each other. In an example, a plurality of conductive hair-penetrating protrusions can be configured in nested (e.g. concentric) rings, wherein each ring comprises multiple protrusions and wherein the rings of protrusions are nested (e.g. concentric) within each other.

In an example, a plurality of conductive hair-penetrating protrusions can be configured in a single circle. In an example, a plurality of conductive hair-penetrating protrusions can be configured in nested circles. In an example, a plurality of conductive hair-penetrating protrusions can be configured in nested (e.g. concentric) circles, wherein each protrusion is ring-shaped and wherein the ring-shaped protrusions are nested (e.g. concentric) within each other. In an example, a plurality of conductive hair-penetrating protrusions can be configured in nested (e.g. concentric) circles, wherein each ring comprises multiple protrusions and wherein the circles of protrusions are nested (e.g. concentric) within each other.

In an example, a plurality of conductive hair-penetrating protrusions can be configured in a single polygon. In an example, a plurality of conductive hair-penetrating protrusions can be configured in nested polygons. In an example, a plurality of conductive hair-penetrating protrusions can be configured in nested (e.g. concentric) polygons, wherein each protrusion is ring-shaped and wherein the ring-shaped protrusions are nested (e.g. concentric) within each other. In an example, a plurality of conductive hair-penetrating protrusions can be configured in nested (e.g. concentric) polygons, wherein each ring comprises multiple protrusions and wherein the polygons of protrusions are nested (e.g. concentric) within each other.

In an example, a plurality of conductive hair-penetrating protrusions can be configured in a square or rectangular grid. In an example, a plurality of conductive hair-penetrating protrusions can be configured in an orthogonal array. In an example, a plurality of conductive hair-penetrating protrusions can be configured in an orthogonal array, wherein a first set of protrusions in the array have a first orientation, wherein a second set of protrusions in the array have a second orientation, and wherein the second orientation is orthogonal (e.g. perpendicular) to the first orientation. In an example, a plurality of conductive hair-penetrating protrusions can be configured in an orthogonal array, wherein a first row of protrusions in the array has a first orientation, wherein a second row of protrusions in the array has a second orientation, and wherein the second orientation is orthogonal (e.g. perpendicular) to the first orientation. In an example, a plurality of conductive hair-penetrating protrusions can be configured in an orthogonal matrix, wherein a row of protrusions in the array has a first orientation, wherein a column of protrusions in the array has a second orientation, and wherein the second orientation is orthogonal (e.g. perpendicular) to the first orientation.

In an example, a plurality of hair-penetrating protrusions in an electromagnetic energy sensor can have different lengths. In an example, protrusions toward the center of the sensor can be longer than protrusions toward the periphery of the sensor. In an example, protrusions in the middle of an array of protrusions can be longer than protrusions toward the edges of the array. In an example, protrusions at one end of a linear array of protrusions can be longer than protrusions at the other end. In an example, a plurality of hair-penetrating protrusions in an electromagnetic energy sensor can have different diameters. In an example, protrusions toward the center of the sensor can be wider than protrusions toward the periphery of the sensor. In an example, protrusions in the middle of an array of protrusions can be wider than protrusions toward the edges of the array. In an example, protrusions at one end of a linear array of protrusions can be wider than protrusions at the other end.

In an example, a plurality of conductive hair-penetrating protrusions can be configured in a linear manner. In an example, a plurality of conductive hair-penetrating protrusions can be configured in a linear manner, wherein each protrusion has a linear shape and wherein the linear protrusions are generally parallel to each other. In an example, a plurality of conductive hair-penetrating protrusions can be configured in a linear manner, wherein each line comprises multiple protrusions and wherein the lines of protrusions are generally parallel.

In an example, a plurality of hair-penetrating protrusions in an electromagnetic energy sensor can have different tapers. In an example, protrusions toward the center of the sensor can be more tapered than protrusions toward the periphery of the sensor. In an example, protrusions in the middle of an array of protrusions can be more tapered than protrusions toward the edges of the array. In an example, protrusions at one end of a linear array of protrusions can be more tapered than protrusions at the other end. In an example, a plurality of hair-penetrating protrusions in an electromagnetic energy sensor can have different lengths. In an example, protrusions toward the center of the sensor can be shorter than protrusions toward the periphery of the sensor. In an example, protrusions in the middle of an array of protrusions can be shorter than protrusions toward the edges of the array. In an example, protrusions at one end of a linear array of protrusions can be shorter than protrusions at the other end.

In an example, an electromagnetic energy sensor can comprise an array of hair-penetrating conductive protrusions, wherein each protrusion in the array has a longitudinal axis (extending toward the person's head) and a longitudinal cross-sectional shape which is shaped like a conic section. In an example, an electromagnetic energy sensor can comprise an array of hair-penetrating conductive protrusions, wherein each protrusion in the array has a longitudinal axis (extending toward the person's head) and a longitudinal cross-sectional shape which is parabolic. In an example, an electromagnetic energy sensor can comprise an array of hair-penetrating conductive protrusions, wherein each protrusion in the array has a longitudinal axis (extending toward the person's head) and a longitudinal cross-sectional shape which is shaped like a golf tee.

In an example, a plurality of hair-penetrating protrusions in an electromagnetic energy sensor can have different diameters. In an example, protrusions toward the center of the sensor can be narrower than protrusions toward the periphery of the sensor. In an example, protrusions in the middle of an array of protrusions can be narrower than protrusions toward the edges of the array. In an example, protrusions at one end of a linear array of protrusions can be narrower than protrusions at the other end. In an example, a plurality of hair-penetrating protrusions in an electromagnetic energy sensor can have different tapers. In an example, protrusions toward the center of the sensor can be less tapered than protrusions toward the periphery of the sensor. In an example, protrusions in the middle of an array of protrusions can be less tapered than protrusions toward the edges of the array. In an example, protrusions at one end of a linear array of protrusions can be less tapered than protrusions at the other end.

In an example, the distance between an electromagnetic energy sensor and the surface of a person's head can be adjusted. In an example, the distance between an electromagnetic energy sensor and the surface of a person's head can be automatically adjusted. In an example, the distance between an electromagnetic energy sensor and the surface of a person's head can be manually adjusted. In an example, the distance between an electromagnetic energy sensor and the surface of a person's head can be adjusted by movement of an electromagnetic actuator. In an example, the distance between an electromagnetic energy sensor and the surface of a person's head can be adjusted by a hydraulic mechanism.

In an example, the distances between a plurality of electromagnetic energy sensors and the surface of a person's head can be individually adjusted. In an example, the distances between a plurality of electromagnetic energy sensors and the surface of a person's head can be individually and automatically adjusted. In an example, the distance between an electromagnetic energy sensor and the surface of a person's head can be adjusted by inflation of a gas-filled chamber. In an example, the distance between an electromagnetic energy sensor and the surface of a person's head can be adjusted by adjusting the tension in a spring, coil, other tensile member, or elastic member.

In an example, the distance between an electromagnetic energy sensor and the surface of a person's head can be adjusted by changing the tension of a spring and/or coil. In an example, the distance between an electromagnetic energy sensor and the surface of a person's head can be adjusted by changing the tension of an elastic strap and/or band. In an example, an electromagnetic energy can be moved closer to the surface of a person's head by a compressible material. In an example, an electromagnetic energy can be moved closer to the surface of a person's head by compressible foam.

In an example, an electromagnetic energy can be moved closer to the surface of a person's head by a hydraulic mechanism. In an example, an electromagnetic energy can be moved closer to the surface of a person's head by a pneumatic mechanism. In an example, an electromagnetic energy can be moved closer to the surface of a person's head by a telescoping mechanism. In an example, an electromagnetic energy can be moved closer to the surface of a person's head by a piston mechanism.

In an example, an electromagnetic energy can be moved closer to the surface of a person's head by a metal spring. In an example, an electromagnetic energy can be moved closer to the surface of a person's head by a plastic spring. In an example, an electromagnetic energy can be moved closer to the surface of a person's head by a leaf spring. In an example, an electromagnetic energy can be moved closer to the surface of a person's head by a helical or spiral spring. In an example, an electromagnetic energy can be moved closer to the surface of a person's head by an adjustable strap or band. In an example, an electromagnetic energy can be moved closer to the surface of a person's head by an electromagnetic mechanism. In an example, an electromagnetic energy can be moved closer to the surface of a person's head by a solenoid mechanism.

In an example, the force with which an electromagnetic energy sensor is pressed against the surface of a person's head can be adjusted. In an example, the force with which an electromagnetic energy sensor is pressed against the surface of a person's head can be automatically adjusted. In an example, the force with which an electromagnetic energy sensor is pressed against the surface of a person's head can be manually adjusted.

In an example, the force with which an electromagnetic energy sensor is pressed against the surface of a person's head can be adjusted by movement of an electromagnetic actuator. In an example, the force with which an electromagnetic energy sensor is pressed against the surface of a person's head can be adjusted by a hydraulic mechanism. In an example, the forces with which a plurality of electromagnetic energy sensors are pressed against the surface of a person's head can be individually adjusted. In an example, the forces with which a plurality of electromagnetic energy sensors are pressed against the surface of a person's head can be individually and automatically adjusted.

In an example, the force with which an electromagnetic energy sensor is pressed against the surface of a person's head can be adjusted by inflation of a gas-filled chamber. In an example, the force with which an electromagnetic energy sensor is pressed against the surface of a person's head can be adjusted by adjusting the tension in a spring, coil, other tensile member, or elastic member. In an example, the force with which an electromagnetic energy sensor is pressed against the surface of a person's head can be adjusted by changing the tension of a spring and/or coil.

In an example, the force with which an electromagnetic energy sensor is pressed against the surface of a person's head can be adjusted by changing the tension of an elastic strap and/or band. In an example, an electromagnetic energy sensor can be pressed against the surface of a person's head by a compressible material. In an example, an electromagnetic energy sensor can be pressed against the surface of a person's head by compressible foam.

In an example, an electromagnetic energy sensor can be pressed against the surface of a person's head by a hydraulic mechanism. In an example, an electromagnetic energy sensor can be pressed against the surface of a person's head by a pneumatic mechanism. In an example, an electromagnetic energy sensor can be pressed against the surface of a person's head by a telescoping mechanism. In an example, an electromagnetic energy sensor can be pressed against the surface of a person's head by a piston mechanism.

In an example, an electromagnetic energy sensor can be pressed against the surface of a person's head by a metal spring. In an example, an electromagnetic energy sensor can be pressed against the surface of a person's head by a plastic spring. In an example, an electromagnetic energy sensor can be pressed against the surface of a person's head by a leaf spring. In an example, an electromagnetic energy sensor can be pressed against the surface of a person's head by a helical or spiral spring. In an example, an electromagnetic energy sensor can be pressed against the surface of a person's head by an adjustable strap or band. In an example, an electromagnetic energy sensor can be pressed against the surface of a person's head by an electromagnetic mechanism. In an example, an electromagnetic energy sensor can be pressed against the surface of a person's head by a solenoid mechanism.

In an example, an electromagnetic energy sensor can be vibrated and/or oscillated in order to penetrate a layer of a person's hair and achieve better electromagnetic communication with a person's brain. In an example, an electromagnetic energy sensor can be vibrated and/or oscillated until it penetrates a layer of a person's hair and achieves a desired level of electromagnetic communication with a person's brain. In an example, a mobile wearable device can include an electromagnetic vibrator which moves hair-penetrating protrusions back and forth to help the protrusions penetrate layers of the person's hair.

In an example, a mobile wearable device can include an electromagnetic vibrator which moves hair-penetrating protrusions back and forth to help the protrusions slide between strands of the person's hair. In an example, when a person first puts on the device, the device can automatically vibrate until good electromagnetic communication is established between the hair-penetrating protrusions and the person's brain. In an example, when a person first puts on the device, the device can automatically vibrate until improvement of electromagnetic communication between the hair-penetrating protrusions and the person's brain stops improving due to vibration. In an example, when a person first puts on the device, the device can automatically vibrate until a selected level of electromagnetic communication between the hair-penetrating protrusions and the person's brain is achieved.

In an example, an electromagnetic energy sensor can have a plurality of protrusions which movably extend toward the surface of a person's head or retract away from the surface of the person's head. In an example, an electromagnetic energy sensor can have a plurality of protrusions which are automatically extended toward the surface of a person's head or retracted away from the surface of the person's head. In an example, an electromagnetic energy sensor can have a plurality of protrusions which are automatically extended toward the surface of a person's head or retracted away from the surface of the person's head so as to automatically adjust the pressure with which the protrusions contact the surface of person's head. In an example, an electromagnetic energy sensor can have a plurality of protrusions which are automatically extended toward the surface of a person's head or retracted away from the surface of the person's head so as to automatically adjust the distance between the protrusions and the surface of person's head.

In an example, an electromagnetic energy sensor can include a gimbal mechanism which maintains a desired angle of contact between the sensor and the surface of a person's head, even if the sensor is moved. In an example, an electromagnetic energy sensor can include a gimbal mechanism which maintains a desired angle of contact between the sensor and the surface of a person's head, even when the person moves.

In an example, an electromagnetic energy sensor can be rotated so as to penetrate a layer of a person's hair and achieve better electromagnetic communication with the person's brain. In an example, an electromagnetic energy sensor can be rotated around an axis which is substantially orthogonal to the surface of a person's head so as to penetrate a layer of a person's hair and achieve better electromagnetic communication with the person's brain. In an example, an electromagnetic energy sensor can be rotated, in an oscillating manner, around an axis which is substantially orthogonal to the surface of a person's head so as to penetrate a layer of a person's hair and achieve better electromagnetic communication with the person's brain.

In an example, a mobile wearable device can include a plurality of electromagnetic actuators which rotate hair-penetrating protrusions back and forth (e.g. clockwise and counter-clockwise) to help the protrusions slide between strands of the person's hair. In an example, when a person first puts on the device, the device can automatically rotate the protrusions back and forth until good electromagnetic communication is established between the hair-penetrating protrusions and the person's brain. In an example, when a person first puts on the device, the device can automatically rotate the protrusions back and forth until improvement of electromagnetic communication between the hair-penetrating protrusions and the person's brain stops improving due to vibration. In an example, when a person first puts on the device, the device can automatically rotate the protrusions back and forth until a selected level of electromagnetic communication between the hair-penetrating protrusions and the person's brain is achieved.

In an example, an electromagnetic energy sensor can be extended toward and retracted away from the surface of a person's head in an oscillating manner so as to penetrate a layer of a person's hair and achieve better electromagnetic communication with the person's brain. In an example, an electromagnetic energy sensor can be vibrated so as to penetrate a layer of a person's hair and achieve better electromagnetic communication with the person's brain. In an example, an electromagnetic energy sensor can be oscillated so as to penetrate a layer of a person's hair and achieve better electromagnetic communication with the person's brain.

Alternatively, some of the designs for electromagnetic energy sensors which have been disclosed herein can be used to deliver electromagnetic energy instead of (or in addition to) sensing electromagnetic energy. In an example, some of the designs for electromagnetic energy sensors which have been disclosed herein can be used for therapeutic neurostimulation applications instead of (or in addition to) EEG sensing applications. In an example, some of the designs for electromagnetic energy sensors which have been disclosed herein can be used to deliver therapeutic electromagnetic energy stimulation instead of (or in addition to) sensing electromagnetic energy. For example, some of the conductive sensor designs disclosed herein can be used for Transcranial Direct Current Stimulation (tDCS) applications instead of (or in addition to) EEG monitoring.

In an alternative embodiment, some of the designs for electromagnetic energy sensors which have been disclosed herein can be part of head-worn mobile neurostimulation device, wherein the designs as used as electrodes which transmit low levels of electromagnetic energy into a person's head for therapeutic purposes. There are numerous potential applications for such a head-worn mobile neurostimulation device including: alleviation of pain; addiction treatment; alleviating headaches; reducing body tremors; reducing seizures; addressing stress and anxiety; treating depression; increasing focus or energy level; reducing hunger; improving sleep; addressing attention-related disorders; and neurological rehabilitation.

In an example, a head-worn mobile neurostimulation device can transmit electromagnetic energy into a person's body in order to modify the electromagnetic activity of their brain. In an example, electromagnetic energy emitters can transmit electromagnetic energy into body tissue in order to modify, adjust, stimulate, and/or selectively block electromagnetic brain activity. In an example, a head-worn mobile neurostimulation device can provide anodal stimulation to excite electromagnetic brain activity. In an example, a head-worn mobile neurostimulation device can provide cathodal stimulation to inhibit electromagnetic brain activity. In an example, a head-worn mobile neurostimulation device can be configured to transmit electromagnetic energy into a particular area of a person's brain. In an example, a head-worn mobile neurostimulation device can transmit electromagnetic energy into a person's body in order to modify the electromagnetic activity of other portions of their nervous system. In an example, a head-worn mobile neurostimulation device can be configured to transmit electromagnetic energy into a selected nerve in the person's head.

In an example, electromagnetic energy which is emitted from an electromagnetic energy emitter can be direct current. In an example a head-worn mobile neurostimulation device can provide Transcranial Direct Current Stimulation (tDCS). In an example a head-worn mobile neurostimulation device can provide Transcranial Electric Stimulation (tES). In an example, electromagnetic energy which is emitted from an electromagnetic energy emitter can be alternating current. In an example a head-worn mobile neurostimulation device can provide Transcranial Alternating Current Stimulation (tACS). In an example, electromagnetic energy which is emitted from an electromagnetic energy emitter can vary between alternating current and direct current or provide a combination of them at the same time. In an example a head-worn mobile neurostimulation device can provide Transcranial Magnetic Stimulation (tMS).

In an example, electromagnetic energy from an electromagnetic energy emitter can have a wave or pulse frequency in the range of 0.1 Hz to 2,500 Hz. In an example, electromagnetic energy from an electromagnetic energy emitter can have a wave or pulse amplitude in the range from 1 μA to 1000 mA. In an example, electromagnetic energy from an electromagnetic energy emitter can have a wave or pulse width in the range of 5 μSec to 500 mSec. In an example, electromagnetic energy from an electromagnetic energy emitter can have an electrical current level in the range from 0.01 mA to 1000 mA. In an example, electromagnetic energy from an electromagnetic energy emitter can have or create an electromagnetic field in the range of 5 V/m to 500 V/m. In an example, electromagnetic energy from an electromagnetic energy emitter can have or create an electromagnetic field gradient of over 1 V/m/mm.

In an example, electromagnetic energy from an electromagnetic energy emitter can have a particular wave form or wave morphology. In an example, electromagnetic energy from an electromagnetic energy emitter can have a sinusoidal wave pattern. In an example, electromagnetic energy from an electromagnetic energy emitter can have a saw tooth wave, square wave, or triangle wave pattern. In an example, electromagnetic energy from an electromagnetic energy emitter can have a biphasic pattern or tri-phasic pattern.

In an example, electromagnetic energy from an electromagnetic energy emitter can comprise signal spikes. In an example, electromagnetic energy from an electromagnetic energy emitter can have pattern randomization or pattern repetition. In an example, electromagnetic energy from an electromagnetic energy emitter can have a selected Fourier transformation or inverse Fourier transformation pattern. In an example, electromagnetic energy from an electromagnetic energy emitter can replicate (imitate) a natural neural transmission signal or be the inverse of a natural neural transmission signal. In an example, electromagnetic energy from an electromagnetic energy emitter can have a selected signal continuity and/or duty cycle. In an example, electromagnetic energy from an electromagnetic energy emitter can have selected signal cycling times. In an example, electromagnetic energy from an electromagnetic energy emitter can have selected signal ramping and/or signal dampening.

I claim:

1. A mobile wearable EEG device comprising:
a forehead band which is configured to span across a person's forehead;
at least one clip, snap, clamp, hook, or plug which connects the forehead band to a longitudinal sidepiece of an eyewear frame; and
at least one electromagnetic energy sensor on the forehead band.

2. The device in claim 1 wherein the forehead band is transparent.

3. The device in claim 1 wherein the forehead band is connected to the middle third of the length of the longitudinal sidepiece of the eyewear frame.

4. The device in claim 1 wherein the connection of a forehead band with the longitudinal sidepiece of the eyewear frame forms a forward-facing acute angle in the range of 20 to 60 degrees.

5. The device in claim 1 wherein the device further comprises a battery, a data processor, a wireless data transmitter, and a wireless data receiver.

6. A mobile wearable EEG device comprising:
an upper loop which is configured to loop over the top of a person's head;
at least one clip, snap, clamp, hook, or plug which connects the upper loop to a longitudinal sidepiece of an eyewear frame; and
at least one electromagnetic energy sensor on the upper loop.

7. The device in claim 6 wherein the upper loop is transparent.

8. The device in claim 6 wherein the upper loop is connected to a middle third of the length of the longitudinal sidepiece of the eyewear frame.

9. The device in claim 6 wherein the upper loop has an undulating shape.

10. The device in claim 6 wherein the upper loop has a forward-facing concavity.

11. The device in claim 6 wherein the at least one electromagnetic energy sensor on the upper loop has protrusions which protrude between strands of hair in order to be in contact the person's scalp and/or be in better electromagnetic communication with the person's brain.

12. The device in claim 11 wherein hair-penetrating protrusions are made from polydimethylsiloxane, polybutylene terephthalate, or polyurethane which has been impregnated, doped, filled, and/or coated with silver, carbon nanotubes or other forms of carbon, copper, aluminum, nickel, platinum, or gold.

13. A mobile wearable EEG device comprising:
an oblong soft pad;
at least one clip, snap, clamp, hook, or plug which connects the oblong soft pad to a longitudinal sidepiece of an eyewear frame; and
at least one electromagnetic energy sensor on the oblong soft pad.

14. The device in claim 13 wherein the oblong soft pad is transparent.

15. The device in claim 13 wherein the oblong soft pad has a rounded rectangular shape.

16. The device in claim 13 wherein the oblong soft pad has an elliptical or oval shape.

17. The device in claim 13 wherein the oblong soft pad has a crescent or boomerang shape.

18. The device in claim 13 wherein a front half of the oblong soft pad is thicker than a rear half of the oblong soft pad.

19. The device in claim 13 wherein a front half of the oblong soft pad is at least twice as thick as a rear half of the oblong soft pad.

20. The device in claim 13 wherein the at least one electromagnetic energy sensor is made from polydimethylsiloxane, polybutylene terephthalate, or polyurethane which has been impregnated, doped, filled, and/or coated with silver, carbon nanotubes or other forms of carbon, copper, aluminum, nickel, platinum, or gold
